(19) United States
(12) Patent Application Publication
(10) Pub. No.: US 2009/0018093 A1
(43) Pub. Date: Jan. 15, 2009

(54) Nucleic Acid Ligands Specific to Immunoglobuline E and Their Use as Atopic Disease Therapeutics

(76) Inventors: Sharon Cload, Cambridge, MA (US); John L. Diener, Cambridge, MA (US); David Epstein, Belmont, MA (US); Jess Wagner-Whyte, Lynn, MA (US)

Correspondence Address: MINTZ, LEVIN, COHN, FERRIS, GLOVSKYAND POPEO, P.C., ONE FINANCIAL CENTER, BOSTON, MA, 02111, US

(21) Appl. No.: 11/885,902

(22) PCT Filed: Dec. 1, 2005
(86) PCT No.: PCT/US2005/043551

§ 371 Date: Jul. 8, 2008

Related U.S. Application Data
(63) Continuation-in-part of application No. 11/115,780, filed on Apr. 26, 2005.
(60) Provisional application No. 60/660,204, filed on Mar. 7, 2005.

(51) Int. Cl. A61K 31/7088; C07H 21/00; C12Q 1/68; C07K 1/00; A61P 37/00

(52) U.S. Cl. 514/44; 536/22.1; 435/6; 530/412

(57) ABSTRACT

The invention discloses aptamers capable of binding to Immunoglobulin E (“IgE”) useful as therapeutics in and diagnostics of atopic disease and/or other diseases or disorders in which IgE has been implicated. The invention further relates to materials and methods for the administration of aptamers capable of binding to IgE.

### FIELD OF INVENTION

The invention relates generally to the field of nucleic acids and more particularly to aptamers capable of binding to Immunoglobulin E (“IgE”) useful as therapeutics in and diagnostics of atopic disease and/or other diseases or disorders in which IgE has been implicated. The invention further relates to materials and methods for the administration of aptamers capable of binding to IgE.

### BACKGROUND OF THE INVENTION

Aptamers are nucleic acid molecules having specific binding affinity to molecules through interactions other than classic Watson-Crick base pairing.

Aptamers, like peptides generated by phage display or monoclonal antibodies (“mAbs”), are capable of specifically binding to selected targets and modulating the target's activity, e.g., through binding aptamers may block their target's ability to function. Created by an in vitro selection process from pools of random sequence oligonucleotides, aptamers have been generated for over 100 proteins including growth factors, transcription factors, enzymes, immunoglobulins, and receptors. A typical aptamer is 10-15 kDa in size (30-45 nucleotides), binds its target with sub-nanomolar affinity, and discriminates against closely related targets (e.g., aptamers will typically not bind other proteins from the same gene family). A series of structural studies have shown that aptamers are capable of using the same types of binding interactions (e.g., hydrogen bonding, electrostatic complementarities, hydrophobic contacts, steric exclusion) that drive affinity and specificity in antibody-antigen complexes.

Aptamers have a number of desirable characteristics for use as therapeutics and diagnostics including high specificity and affinity, biological efficacy, and excellent pharmacokinetic properties. In addition, they offer specific competitive advantages over antibodies and other protein biologics, for example:

1) Speed and control. Aptamers are produced by an entirely in vitro process, allowing for the rapid generation of initial leads, including therapeutic leads. In vitro selection allows the specificity and affinity of the aptamer to be tightly controlled and allows the generation of leads, including leads against both toxic and non-immunogenic targets.

2) Toxicity and Immunogenicity. Aptamers as a class have demonstrated little or no toxicity or immunogenicity. In chronic dosing of rats or woodchucks with high levels of aptamer (10 mg/kg daily for 90 days), no toxicity is observed by any clinical, cellular, or biochemical measure. Whereas the efficacy of many monoclonal antibodies can be severely limited by immune response to antibodies themselves, it is extremely difficult to elicit antibodies to aptamers most likely because aptamers cannot be presented by T-cells via the MHC and the immune response is generally trained not to recognize nucleic acid fragments.

3) Administration. Whereas most currently approved antibody therapeutics are administered by intravenous infusion (typically over 2-4 hours), aptamers can be administered by subcutaneous injection (aptamer bioavailability via subcutaneous administration is >80% in monkey studies (Tucker et al., J. Chromatography B. 732: 203-212, 1999)). This difference is primarily due to the comparatively low solubility and thus large volumes necessary for most therapeutic mAbs. With good solubility (>150 mg/mL) and comparatively low molecular weight (aptamer: 10-50 kDa; antibody: 150 kDa), a weekly dose of aptamer may be delivered by injection in a volume of less than 0.5 mL. In addition, the small size of aptamers allows them to penetrate into areas of conformational constrictions that do not allow for antibodies or antibody fragments to penetrate, presenting yet another advantage of aptamer-based therapeutics or prophylaxis.

4) Scalability and cost. Therapeutic aptamers are chemically synthesized and consequently can be readily scaled as needed to meet production demand. Whereas difficulties in scaling production are currently limiting the availability of some biologics and the capital cost of a large-scale protein production plant is enormous, a single large-scale oligonucleotide synthesizer can produce upwards of 100 kg/year and requires a relatively modest initial investment. The current cost of goods for aptamer synthesis at the kilogram scale is estimated at $500/g, comparable to that for highly optimized antibodies. Continuing improvements in process development are expected to lower the cost of goods to <$100/g in five years.

5) Stability. Therapeutic aptamers are chemically robust. They are intrinsically adapted to regain activity following exposure to factors such as heat and denaturants and can be stored for extended periods (>1 yr) at room temperature as lyophilized powders.

### IgE and Atopic Disease

Atopy is the genetic predisposition to produce allergen-specific IgE and is one of the most important predisposing factors for the development of asthma and other allergic diseases. Atopic diseases such as allergic rhinitis (hayfever), asthma, and atopic dermatitis are prevalent among the U.S. population and are on the rise. Symptoms of allergic disease include vasodilation, smooth muscle contraction, local inflammation and vascular permeability. Increased production of IgE in response to common environmental allergens is the hallmark of atopic disease. Common allergens include dustmite feces, pollen, foods, animal dander and fungal spores. Mast cells are known to play a central role in the immediate phase reaction of allergic diseases through IgE-mediated release of a variety of chemical mediators like histamine, leukotrienes, and prostaglandins. T lymphocytes, basophils and eosinophils are thought to be responsible in inducing the late phase response. Immediate hypersensitivity caused by the stimulation of mast cells and basophils upon contact of allergy-specific IgE with antigen is a powerful mammalian immune effector system. These IgE-mediated reactions can cause diseases such as allergic rhinitis, atopic dermatitis, urticaria, food allergies, asthma, and in the most severe cases, anaphylactic shock which can result in death.

While not intending to be bound by any theory, the mechanism whereby IgE mediates allergic responses has been determined. In brief, IgE binds to the a chain of the high affinity IgE receptor, FcεRI, on mast cells and basophils. FcεRI on these cell types is tetrameric consisting of an α chain, a β chain and homodimeric γ chains. The β and γ chains are the signal transducing domains of FcεRI. Allergen cross-linking of IgE bound to mast cells results in stimulation of FcεRI and activation of a number of signal transduction pathways that lead to the release of a range of proinflammatory mediators and cytokines, including bronchoconstrictive and vasoactive substances, such as histamine, leukotrienes and various other cytokines (see FIG. 1). The role of IgE and Fc_{ε}RI and mast cells has been confirmed in animal models of anaphylaxis: systemic delivery of IgE plus specific antigen (or treatment with anti-IgE alone) causes anaphylactic reactions in normal mice but fails to trigger immediate systemic responses in mast-cell-deficient or Fc_{ε}RI-deficient mice.

Currently, there are therapeutic approaches under clinical evaluation that interfere with the immunological mechanisms underlying allergen-induced pathology, for example, an anti-IgE immunoglobulin, which directly targets IgE serum antibodies, thus inhibiting the central mechanism of immediate type hypersensitivity reactions. In addition, there has been interest in developing allergen-specific immunotherapy due to its potential to cure allergic diseases. However, both anti-IgE immunoglobulins and allergen-specific immunotherapy are limited by high costs and the necessity for permanent or every-season treatment.

In addition to the previously described advantages of aptamers as a novel class of therapeutics, because aptamers are nucleic acids, they can incorporate motifs that have an immuno-stimulatory effect desirable and beneficial in a therapeutic for atopic and other immune diseases. These motifs include the CpG motifs that have immunomodulatory effects, such as suppression of allergic responses mediated by type II T helper (T_{H}2) cells. CpG has been shown to rapidly induce expression of T-bet mRNA in purified B cells (Liu et al., 2003, Nature Immun. Vol 4, no. 7, p. 687-693).

Thus, it would be beneficial to have materials and methods to disrupt the biological function of IgE to treat disease in which it is implicated in pathogenesis. The present invention provides materials and methods to meet these and other needs.

### SUMMARY OF THE INVENTION

The present invention provides materials and methods for the treatment, prevention and/or amelioration of atopic disease. In one embodiment, an aptamer comprising a nucleic acid sequence selected from the group consisting of: SEQ ID NOS 11-15, 18, 19, 21, 29, 33, 41-44, 46,50,56-96, 98-102, 119-124, 126-136, 139-157, 158-176, 178-190, 194-201, 206-243, 247, 249-259, 261-267, 269-290, 292, and 299-336 conjugated to a PEG moiety is provided. In a particular embodiment, an aptamer comprising the following sequence: nAmGmCmCmUdGmGdG-s-dGmAmCmCmCmAmU-s-dI-s-mGdI-s-dGdI-s-dGmCmU (SEQ ID NO 298) conjugated to a PEG moiety is provided. In some embodiments, the PEG moiety comprises a molecular weight selected from the group consisting of: 60 kDa, 40 kDa, 30 kDa and 20 kDa. In some embodiments, the PEG moiety is a branched while in other embodiments it is linear.

In a particular embodiment, the aptamer of the invention comprises the structure set forth below:

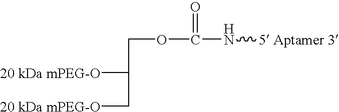

where,

[symbol] indicates a linker

(SEQ ID NO 216)

Aptamer = mAmGmCmCmUdGmGdG-s-dGmACmCmCmAmU-s-dI-s-
mGdI-s-dGdI-s-dGmCmU-3T

wherein in mC, mG, mU and mA=2′-OMe C, 2′-OMe G, 2′-OMe U and 2′-OMe A respectively, dG=deoxy G, dI=deoxy inosine, s=a phosphorothioate backbone substitution, and 3T=3′ inverted deoxy thymidine.

In some embodiments, the aptamer of the invention comprises the structure set forth below:

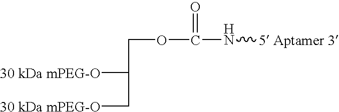

where,

[symbol] indicates a linker

(SEQ ID NO 216)

Aptamer = mAmGmCmCmUdGmGdG-s-dGmACmCmCmAmU-s-dI-s-
mGdI-s-dGdI-s-dGmCmU-3T

wherein mC, mG, mU and mA=2′-OMe C, 2′-OMe G, 2′-OMe U and 2′-OMe A respectively, dG=deoxy G, dI=deoxy inosine, s=a phosphorothioate backbone substitution, and 3T=3′ inverted deoxy thymidine.

In some embodiments, the aptamer of the invention comprises the structure set forth below:

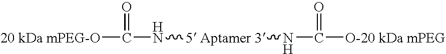

where,

[symbol] indicates a linker

(SEQ ID NO 298)

Aptamer = mAmGmCmCmUdGmGdG-s-dGmAmCmCmCmAmU-s-dI-
s-mGdI-s-dGdI-s-dGmCmU

wherein mC, in G, mU and mA=2′-OMe C, 2′-OMe G, 2′-OMe U and 2′-OMe A respectively, dG=deoxy G, dI=deoxy inosine, s=a phosphorothioate backbone substitution, and 3T=3′ inverted deoxy thymidine.

In some embodiments, the aptamer of the invention comprises the structure set forth below:

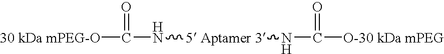

where,

[symbol] indicates a linker

(SEQ ID NO 298)

Aptamer = mAmGmCmCmUdGmGdG-s-dGmAmCmCmCmAmU-s-dI-
s-mGdI-s-dGdI-s-dGmCmU

wherein mC, mG, mU and mA=2′-OMe C, 2′-OMe G, 2′-OMe U and 2′-OMe A respectively, dG=deoxy G, dI=deoxy inosine, s=a phosphorothioate backbone substitution, and 3T=3′ inverted deoxy thymidine.

In some embodiments the aptamer of the invention comprises a non-alkyl linker. In some embodiments the aptamer of the invention comprises an alkyl linker. In some embodiments, the alkyl linker comprises 2 to 18 consecutive CH₂ group, particularly 2 to 12 consecutive CH₂ groups and more particularly 3 to 6 consecutive CH₂.

In some embodiments, the aptamer of the invention comprises the structure set forth below:

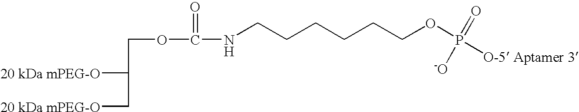

(SEQ ID NO 216)

Aptamer = mAmGmCmCmUdGmGdG-s-dGmACmCmCmAmU-s-dI-s-
mGdI-s-dGdI-s-dGmCmU-3T

wherein mC, mG, in U and mA=2′-OMe C, 2′-OMe G, 2′-OMe U and 2′-OMe A respectively, dG=deoxy G, dI=deoxy inosine, s=a phosphorothioate backbone substitution, and 3T=3′ inverted deoxy thymidine.

In some embodiments, the aptamer of the invention comprises the structure set forth below:

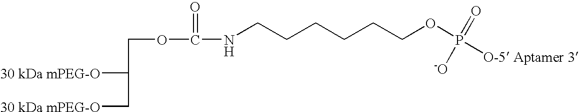

(SEQ ID NO 216)

Aptamer = mAmGmCmCmUdGmGdG-s-dGmACmCmCmAmU-s-dI-s-
mGdI-s-dGdI-s-dGmCmU-3T

wherein mC, mG, mU and mA=2′-OMe C, 2′-OMe G, 2′-OMe U and 2′-OMe A respectively, dG=deoxy G, dI=deoxy inosine, s=a phosphorothioate backbone substitution, and 3T=3′ inverted deoxy thymidine.

In some embodiments, the aptamer of the invention comprises the structure set forth below:

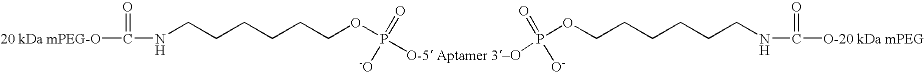

(SEQ ID NO 298)

Aptamer = mAmGmCmCmUdGmGdG-s-dGmAmCmCmCmAmU-s-dI-
s-mGdI-s-dGdI-s-dGmCmU

wherein mC, mG, mU and mA=2′-OMe C, 2′-OMe G, 2′-OMe U and 2′-OMe A respectively, dG=deoxy G, dI=deoxy inosine, s=a phosphorothioate backbone substitution, and 3T=3′ inverted deoxy thymidine.

In some embodiments, the aptamer of the invention comprises the structure set forth below:

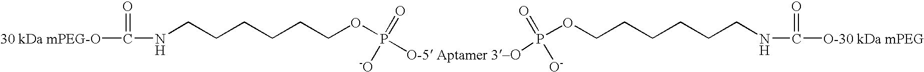

(SEQ ID NO 298)

Aptamer = mAmGmCmCmUdGmGdG-s-dGmAmCmCmCmAmU-s-dI-
s-mGdI-s-dGdI-s-dGmCmU

wherein mC, mG, mU and mA=2′-OMe C, 2′-OMe G, 2′-OMe U and 2′-OMe A respectively, dG=deoxy G, dI=deoxy inosine, s=a phosphorothioate backbone substitution, and 3T=3′ inverted deoxy thymidine.

In some embodiments, the PEGylated aptamer of the invention modulates, particularly inhibits, a function of IgE or a variant thereof. In some embodiments, the PEGylated aptamer inhibits a function of IgE or a variant thereof in vitro. In some embodiments, the PEGylated aptamer inhibits a function of IgE or a variant thereof in vivo. In some embodiments, the PEGylated aptamer of the invention prevents binding of IgE to its receptor. In some embodiments, a method of treating, preventing and/or ameliorating a disease mediated by IgE, comprising administering an effective amount of a PEGylated aptamer of the invention or a salt thereof to a vertebrate, preferably a mammal, more preferably a human, is provided.

In some embodiments the invention provides a therapeutic composition comprising a therapeutically effective amount of any of the PEGylated aptamers of the invention or a salt thereof. In some embodiments the therapeutic composition further comprises a pharmaceutically acceptable carrier or diluent.

In one embodiment, a method of treating a disease mediated by IgE, comprising administering an effective amount of a PEGylated aptamer of the invention, particularly administering the therapeutic composition, to a vertebrate, preferably to a mammal, particularly to a human is provided. In some embodiments the disease to be treated is atopic disease. In particular embodiments the disease to be treated is selected from the group consisting of: allergic rhinitis, atopic dermatitis, asthma, acute urticaria, food allergies, peanut allergy, systemic anaphylaxis, allergic conjunctivitis, vernal keratoconjunctivitis, atopic keratoconjunctivitis, giant papillary conjunctivitis, and eosinophilic gastroenteritis. In a particular embodiment, the disease to be treated is asthma.

In some embodiments, an effective amount of a PEGylated anti-IgE aptamer of the invention is administered to a subject, such as a vertebrate, preferably a mammal, more preferably a human, in conjunction with an immunostimulatory nucleic acid sequence, such as a second aptamer comprising a CpG motif.

In some embodiments the PEGylated aptamer is administered to the subject via a route selected from the group consisting of subcutaneous administration, intravenous administration and intranasal administration. In a particular embodiment, the therapeutic composition is administered subcutaneously to a human subject having or at risk for atopic disease.

In some embodiments, a diagnostic method comprising contacting a PEG conjugated aptamer of the invention with a composition suspected of comprising IgE or a variant thereof and detecting the presence or absence of IgE or a variant thereof is provided. In some embodiments, a method of detecting IgE in a sample comprising the steps of contacting the sample with a PEGylated aptamer of the invention and determining whether the aptamer specifically binds to the sample, wherein in binding of the aptamer to the sample indicates the sample contains IgE is provided. In some embodiments, an aptamer of the invention for use as an in vitro diagnostic is provided. While in other embodiments, an aptamer of the invention for use as an in vivo diagnostic is provided. In some embodiments an aptamer of the invention for use in the treatment, prevention and/or amelioration of disease in vivo is provided.

In some embodiments, an aptamer that specifically binds to IgE comprising a nucleic acid sequence at least 80% identical, particularly at least 90%, more particularly at least 95% identical to any one of the sequences selected from the group consisting of: SEQ ID NOS 11-15, 18, 19, 21, 29, 33, 41-44, 46, 50, 56-96, 98-102, 201-219, and 299-336 is provided.

In some embodiments, an aptamer that specifically binds to IgE comprising a nucleic acid sequence at least 80% identical, particularly at least 90% identical, more particularly at least 95% identical to the unique sequence region of any one of the sequences selected from the group consisting of: SEQ ID NOS 11-15, 18, 19, 21, 29, 33, 41-44, 46, 50 and 56-89 is provided.

In some embodiments, an aptamer capable of binding IgE comprising a sequence of 30 contiguous nucleotides that are identical to a sequence of 30 contiguous nucleotides in any one of the aptamer acid sequences selected from the group of: SEQ ID NOS 11-15, 18, 19, 21, 29, 33, 41-44, 46, 50 and 56-96 is provided. In particular embodiments, an aptamer comprising 20 contiguous nucleotides that are identical to a sequence of 20 contiguous nucleotides in the unique sequence region of any one of the aptamer sequences selected from the group of: SEQ ID NOS 11-15, 18, 19, 21, 29, 33, 41-44, 46, 50, 56-96, 98-102, 201-219, and 299-336 is provided. In even more particular embodiments, an aptamer comprising 8 contiguous nucleotides that are identical to a sequence of 8 contiguous nucleotides in the unique sequence region of any one of the aptamer sequences selected from the group consisting of: SEQ ID NOS 11-15, 18, 19, 21, 29, 33, 41-44, 46, 50, 56-96, 98-102 is provided. Preferably, the aptamer comprising 8 contiguous nucleotides identical to the unique sequence of an aptamer sequence selected from the group consisting of: SEQ ID NOS 11-15, 18, 19, 21, 29, 33, 41-44, 46, 50, 56-96, 98-102, 201-219, and 299-336 specifically binds to IgE, preferably human IgE and in some embodiments modulates a function of IgE, preferably of human IgE. In some embodiments, an aptamer selected from the group consisting of: SEQ ID NOS 11-15, 18, 19, 21, 29, 33, 41-44, 46, 50, 56-96, 98-102, 119-124, 126-136, 139-157, 158-176, 178-190, 194-201, 206-243, 247, 249-259, 261-267, 269-290, 292, and 299-336 is provided.

In some embodiments, the aptamer of this aspect of the invention is a single stranded nucleic acid. In some embodiments, the aptamer of this aspect conjugated to a high molecular weight, non-immunogenic compound or a lipophilic compound. In some embodiments, the aptamer of this aspect is conjugated to a polyalkylene glycol moiety, particularly a polyethylene glycol moiety. In some embodiments, the polyethylene glycol moiety is branched while in other embodiments it is linear.

In some embodiments, the aptamer of this aspect of the invention comprises a chemical modification selected from the group consisting: of a chemical substitution at a sugar position; a chemical substitution at a phosphate position; a chemical substitution at a base position of the nucleic acid; 3′ capping with an inverted nucleotide, and 5′ capping with an inverted nucleotide. In some embodiments, the aptamer of the invention further comprises an immunostimulatory nucleic acid sequence, such as a CpG motif.

In some embodiments, the aptamer of the invention modulates, particularly inhibits, a function of IgE or a variant thereof. In some embodiments, the aptamer inhibits a function of IgE or a variant thereof in vitro. In some embodiments, the aptamer inhibits a function of IgE or a variant thereof in vivo. In some embodiments, the aptamer of the invention prevents binding of IgE to its receptor. In some embodiments, a method of treating, preventing and/or ameliorating a disease mediated by IgE, comprising administering an effective amount of an aptamer of the invention or a salt thereof to a vertebrate, preferably a mammal, more preferably a human in need thereof, is provided.

In some embodiments, a therapeutic composition comprising a therapeutically effective amount of an aptamer of the invention or a salt thereof, and a pharmaceutically acceptable carrier or diluent is provided. In some embodiments, a method of treating, preventing and/or ameliorating a disease mediated by IgE, comprising administering an effective amount of an aptamer of the invention or a salt thereof, preferably the therapeutic composition of the invention to a subject in need thereof is provided. In some embodiments the subject is a vertebrate, preferably a mammal, more preferably a human.

In some embodiments of the invention, the disease to be treated is an atopic disease, particularly a disease selected from the group consisting of: allergic rhinitis, atopic dermatitis, asthma, acute urticaria, food allergies, peanut allergy, systemic anaphylaxis, allergic conjunctivitis, vernal keratoconjunctivitis, atopic keratoconjunctivitis, giant papillary conjunctivitis, and eosinophilic gastroenteritis.

In some embodiments, the anti-IgE aptamer of the invention is administered to a subject in need thereof, such as a vertebrate, preferably a mammal, more preferably a human, in conjunction with an immunostimulatory nucleic acid sequence, such as a second aptamer comprising a CpG motif. In some embodiments the immunostimulatory nucleic acid sequence is incorporated into or appended to the anti-IgE aptamer of the invention.

In some embodiments the aptamer of the invention is administered to a subject by a route selected from the group consisting of: subcutaneous administration, intravenous administration and intranasal administration.

In some embodiments, a diagnostic method comprising contacting an aptamer of this aspect of the invention with a composition suspected of comprising IgE or a variant thereof and detecting the presence or absence of IgE or a variant thereof is provided. In some embodiments, an aptamer of the invention for use as an in vitro diagnostic is provided. While in other embodiments, an aptamer of the invention for use as an in vivo diagnostic is provided. In some embodiments an aptamer of the invention for use in the treatment, prevention and/or amelioration of disease in vivo is provided.

In some embodiments, a method of detecting IgE in a sample comprising the steps of contacting the sample with an aptamer of this aspect of the invention and determining whether the aptamer specifically binds to the sample, wherein in binding of the aptamer to the sample indicates the sample contains IgE is provided. In some embodiments of this aspect of the invention, the method comprises contacting an aptamer having a nucleic acid sequence selected from the group consisting of: SEQ ID NOS 11-15, 18, 19, 21, 29, 33, 41-44, 46, 50, 56-96, 98-102, 119-124, 126-136, 139-157, 158-176, 178-190, 194-201, 206-243, 247, 249-259, 261-267, 269-290, 292, and 299-336 is provided.

In some embodiments, a method of manufacturing an aptamer of this aspect of the invention is provided, comprising the steps of chemically synthesizing the aptamer and purifying the aptamer. In some embodiments, the manufacturing method further comprises the step of conjugating a polyethylene glycol to the nucleic acid sequence prior to the purification step. In some embodiments of this aspect of the invention, the method comprises manufacturing an aptamer having a nucleic acid sequence selected from the group consisting of: SEQ ID NOS 11-15, 18, 19, 21, 29, 33, 41-44, 46, 50, 56-96, 98-102, 119-124, 126-136, 139-157, 158-176, 178-190, 194-201, 206-243, 247, 249-259, 261-267, 269-290, 292, and 299-336.

In another aspect of the invention, a method for increasing the binding affinity of an aptamer for a target, wherein the aptamer is capable of forming multimeric aggregates, is provided. In one embodiment, the method comprises the step of substituting a nucleotide in the aggregate forming aptamer with a nucleotide selected to prevent aggregate formation whereby the binding affinity of the resulting substituted aptamer for its target is increased relative the binding affinity a parent aptamer, the parent aptamer having the same nucleic acid sequence but lacking the nucleotide substitution. In some embodiments the nucleotide selected to prevent aggregate formation is a modified nucleotide. In some embodiments, the modified nucleotide is inosine.

In another aspect of the invention, a method for increasing the binding affinity of an aptamer for a target is provided, comprising the step of substituting an inosine for at least one nucleotide at a position that increases the binding affinity of the inosine substituted aptamer to the target relative to the binding affinity of the parent aptamer to the same target, the parent aptamer having the same nucleotide sequence but lacking the inosine modification. In one embodiment of the provided method, the substitution step comprises substituting no more than four, three or two inosines for four three or two nucleotides respectively, wherein the resulting aptamer comprises increased binding affinity to the target relative to that of the parent aptamer. In some embodiments, the inosine substituted nucleotide is a purine. In a particular embodiment, the inosine substituted purine is guanosine. In another embodiment, the method of the invention comprises a second chemical substitution step selected from the group consisting of: a chemical substitution at a sugar position; a chemical substitution at a phosphate position and chemical substitution at a base position of the nucleic acid. In a particular, embodiment further substituted aptamer comprises an increased binding affinity to the target relative to an aptamer identical to the further substituted aptamer except that it lacks the second chemical substitution.

In some embodiments, the substituting step comprises chemically synthesizing the aptamer with the desired substitution.

In some embodiments the binding affinity of the inosine substituted aptamer for the target is increased at least two, at least five, at least 10, at least 25, at least 50, at least 75, at least 85, at least 95, at least 100, at least 150, at least 200 fold relative to the parent aptamer. In some embodiments, the substituting step comprises chemically synthesizing the aptamer.

In one aspect of the invention, an aptamer having increased binding affinity for its target obtained by a substitution method of the invention is provided. In a particular embodiment the aptamer comprises increased binding affinity for IgE, particularly human IgE.

In a particular embodiment, an aptamer that binds to IgE comprising a nucleic acid sequence having a 3′ and 5′ end selected from the group consisting of SEQ ID NOs 299-336 is provided. In some embodiments of this aspect of the invention the selected aptamer nucleic acid sequence is further modified to comprise at least one chemical modification. In some embodiments, the chemical modification of the selected aptamer is selected from the group consisting of a chemical substitution at sugar position; a chemical substitution at a phosphate position; and a chemical substitution at a base position of the nucleic acid. In some embodiments, the chemical modification of the selected aptamer is selected from the group consisting of: incorporation of a modified nucleotide, 3′ capping, conjugation to a high molecular weight non-immunogenic compound, and conjugation to a liphophilic compound. In some embodiments, the chemical modification comprises conjugation to a high molecular weight, non-immunogenic compound, particularly polyalkylene glycol, more particularly polyethylene glycol. In some embodiments, the selected aptamer nucleic acid sequence comprises a polyethylene glycol conjugated to its 5′ end. In some embodiments, the selected aptamer nucleic acid sequence comprises a polyethylene glycol conjugated to its 3′ end. In some embodiments, the selected aptamer nucleic acid sequence comprises a polyethylene glycol conjugated to its 5′ and 3′ end. In some embodiments, the selected aptamer nucleic acid sequence comprises a polyethylene glycol having a molecular weight selected from the group consisting of: a 60 kDa, a 40 kDa, a 30 kDa and a 20 kDa. In some embodiments of this aspect of the invention, the polyethylene glycol is linear. In some embodiments of this aspect of the invention, the polyethylene glycol is branched.

In some embodiments, a composition comprising a therapeutically effective amount of an aptamer of this aspect of the invention or a salt thereof is provided. In some embodiments the composition further comprises a pharmaceutically acceptable carrier or diluent. In some embodiments of this aspect of the invention, the composition comprises an aptamer having a nucleic acid sequence selected from the group consisting of SEQ ID NOs 299-336.

In some embodiments, a method of treating atopic disease comprising administering an effective amount of an aptamer of this aspect of the invention or a salt thereof to a subject having atopic disease is provided. In some embodiments, a method of treating a disease mediated by IgE comprising administering an effective amount of the aptamer of this aspect of the invention or salt thereof to a patient having an IgE mediated disease is provided. In some embodiments of this aspect of the invention, the method comprises administering an aptamer having a nucleic acid sequence selected from the group consisting of SEQ ID NOs 299-336.

In some embodiments, a method of detecting IgE in a sample comprising the steps of contacting the sample with an aptamer of this aspect of the invention and determining whether the aptamer specifically binds to the sample, wherein in binding of the aptamer to the sample indicates the sample contains IgE is provided. In some embodiments of this aspect of the invention, the method comprises contacting an aptamer having a nucleic acid sequence selected from the group consisting of SEQ ID NOs 299-336 is provided.

In some embodiments, a method of manufacturing an aptamer of this aspect of the invention is provided, comprising the steps of chemically synthesizing the aptamer and purifying the aptamer. In some embodiments, the manufacturing method further comprises the step of conjugating a polyethylene glycol to the nucleic acid sequence prior to the purification step. In some embodiments of this aspect of the invention, the method comprises manufacturing an aptamer having a nucleic acid sequence selected from the group consisting of SEQ ID NOs 299-336 is provided.

### BRIEF DESCRIPTION OF THE FIGURES

FIG. 1 is a schematic of IgE-mediated signal transduction events.

FIG. 2 is a schematic representation of the in vitro aptamer selection (SELEX™) process from pools of random sequence oligonucleotides comprised of ribonucleic acids. For the in vitro aptamer SELEX™ process using pools of random sequence oligonucleotides comprised of deoxyribonucleic acids, the reverse transcription and transcription steps are omitted.

FIG. 3 is an illustration of a 40 kDa branched PEG.

FIG. 4 is an illustration of a 40 kDa branched PEG attached to the 5′ end of an aptamer.

FIG. 5 is an illustration depicting various PEGylation strategies representing standard mono-PEGylation, multiple PEGylation, and dimerization via PEGylation.

FIG. 6 is a plot of pool binding activity to h-IgE after rounds 6 and 7 of dRmY clone selection.

FIG. 7 shows the direct binding curves and binding affinities for ARC445 (SEQ ID NO 101) and derivatives thereof, depicting that modification yielded increased proportion binding to h-IgE.

FIG. 8 depicts the ion exchange HPLC trace analysis of anti-IgE aptamer ARC445 (SEQ ID NO 101) and several derivatives thereof.

FIG. 9 is a graph showing an increase of NMM fluorescence in ARC445 (SEQ ID NO 101), and a decrease in NMM fluorescence in ARC445 derivatives, ARC909-911 (SEQ ID NOs 191-193), which contain 7-deaza-G substitutions for dG.

FIG. 10 is a graph showing an increase of NMM fluorescence in ARC183, (positive experimental control) and a decrease in NMM fluorescence in ARC1346 (negative experimental control).

FIG. 11 shows the direct binding curves for ARC445 (SEQ ID NO 101) compared to derivatives thereof, depicting that substituting dG with 7-deaza-G (as in ARC909-911 (SEQ ID NOs 191-193)) significantly reduces proportion binding to h-IgE.

FIG. 12 is a graph showing a decrease in NMM fluorescence in ARC445 derivatives ARC1641, 1642, and 1666 (SEQ ID NOs 212, 213, and 216 respectively), which contain inosine substitutions for dG, as compared to ARC1384 (SEQ ID NO 181), an ARC445 derivative containing 2′-O-methyl and phosphorothioate substitutions but no inosine substitutions for dG. (SEQ ID NO 101).

FIG. 13 is a graph depicting the activity of ARC1666 3′-truncants to ARC1666 in an IgE:FcεR1 binding inhibition FACS assay. Percent inhibition is plotted on the vertical axis versus aptamer concentration on the horizontal axis.

FIG. 14 is a schematic of predicted secondary structures for rRfY, dRmY and DNA minimized aptamers showing highest potency in IgE:FcεR1 binding inhibition by FACS. FIG. 14(A) shows the rRfY clone according to SEQ ID NO 91, outlined residues are 2′-F; FIG. 14(B) shows ARC445 (SEQ ID NO 101), a dRmY clone, black residues are 2′-deoxy, grey residues are 2′-OMe; FIG. 14(C) shows ARC475 (SEQ ID NO 151), DNA clone, underlined residues are 2′-deoxy.

FIG. 15 is a plot showing ARC445 (SEQ ID NO 101) and ARC656 (SEQ ID NO 157) blocking of h-IgE-induced histamine release in SX38 cells.

FIG. 16A is a graph depicting the % full length ARC1384 and ARC1666 present in human and rat plasma as a function of incubation time. FIG. 16B is a graph depicting the % full length ARC1384, ARC1572 and ARC1573 present in human and rat plasma as a function of incubation time.

FIG. 17 is a table outlining the design of a pharmacokinetics study of PEGylated anti-IgE aptamers ARC1785 (SEQ ID NO 295), ARC1787 (SEQ ID NO 293), ARC1788 (SEQ ID NO 294), and ARC1790 (SEQ ID NO 296) administered intravenously to mice at 10 mg/kg.

FIG. 18 is a graph showing the pharmacokinetic profile of PEGylated anti-IgE aptamers ARC1785 (SEQ ID NO 295), ARC1787 (SEQ ID NO 293), ARC1788 (SEQ ID NO 294), ARC1790 (SEQ ID NO 296) after intravenous (IV) administration to mice at 10 mg/kg.

FIG. 19 is a table outlining the design of a pharmacokinetics study of PEGylated anti-IgE aptamers ARC1785 (SEQ ID NO 295), ARC1-787 (SEQ ID NO 293), ARC1788 (SEQ ID NO 294), and ARC1790 (SEQ ID NO 296) administered sub-cutaneously to mice at 10 mg/kg.

FIG. 20 is a graph showing the pharmacokinetic profile of PEGylated anti-IgE aptamers ARC1785 (SEQ ID NO 295), ARC1787 (SEQ ID NO 293), ARC1788 (SEQ ID NO 294) and ARC1790 (SEQ ID NO 296) after subcutaneous (SC) administration to mice at 10 mg/kg.

FIG. 21 is a table summarizing the non-compartmental PK parameter estimates for PEGylated anti-IgE aptamers ARC1785 (SEQ ID NO 295), ARC1787 (SEQ ID NO 293), ARC1788 (SEQ ID NO 294) and ARC1790 (SEQ ID NO 296) after intravenous (IV) and subcutaneous (SC) administration to mice at 10 mg/kg.

FIG. 22 is a table outlining the design of an ex vivo study of subcutaneous and intravenous administration of PEGylated anti-IgE aptamers ARC1785 (SEQ ID NO 295), ARC1787 (SEQ ID NO 293), ARC1788 (SEQ ID NO 294), and ARC1790 (SEQ ID NO 296) to cynomolgus macaque at 5 mg/kg.

FIGS. 23 A-D are graphs depicting the concentration-time profiles of ARC1785 (SEQ ID NO 295), ARC1787 (SEQ ID NO 293), ARC1788 (SEQ ID NO 294), and ARC1790 (SEQ ID NO 296) after subcutaneous (SC) and intravenous (IV) administration to cynomolgus macaque at 5 mg/kg. FIGS. 23A, 23B and 23D show the time concentration profiles of 3 animals for both SC and IV administration, and FIG. 23D shows the time concentration profiles of 2 animals for IV administration and 1 animal for SC administration.

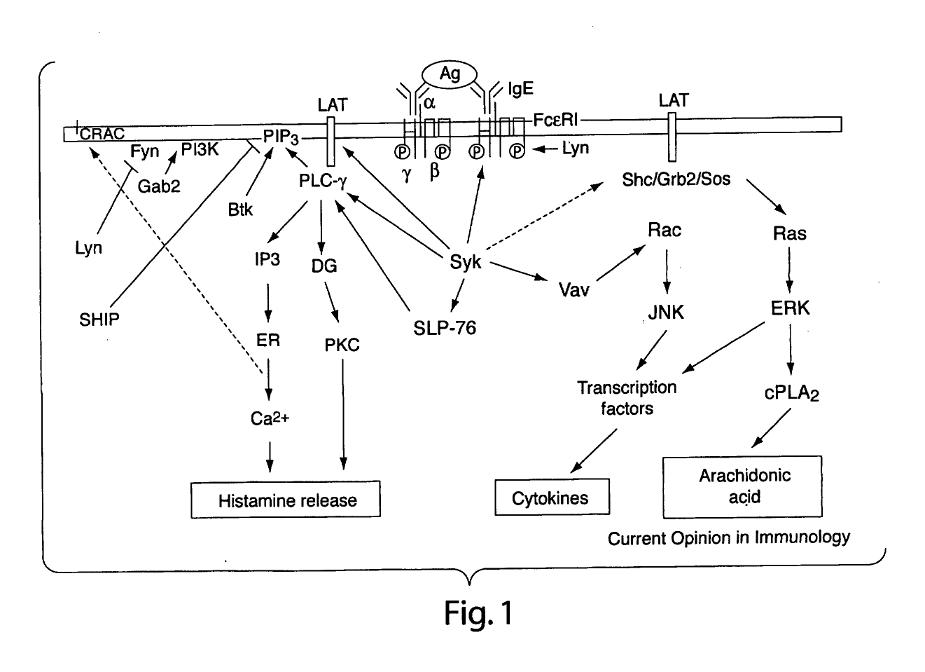

### DETAILED DESCRIPTION OF THE INVENTION

The details of one or more embodiments of the invention are set forth in the accompanying description below. Although any methods and materials similar or equivalent to those described herein can be used in the practice or testing of the present invention, the preferred methods and materials are now described. Other features, objects, and advantages of the invention will be apparent from the description. In the specification, the singular forms also include the plural unless the context clearly dictates otherwise. Unless defined otherwise, all technical and scientific terms used herein have the same meaning as commonly understood by one of ordinary skill in the art to which this invention belongs. In the case of conflict, the present Specification will control.

### The SELEX™ Method

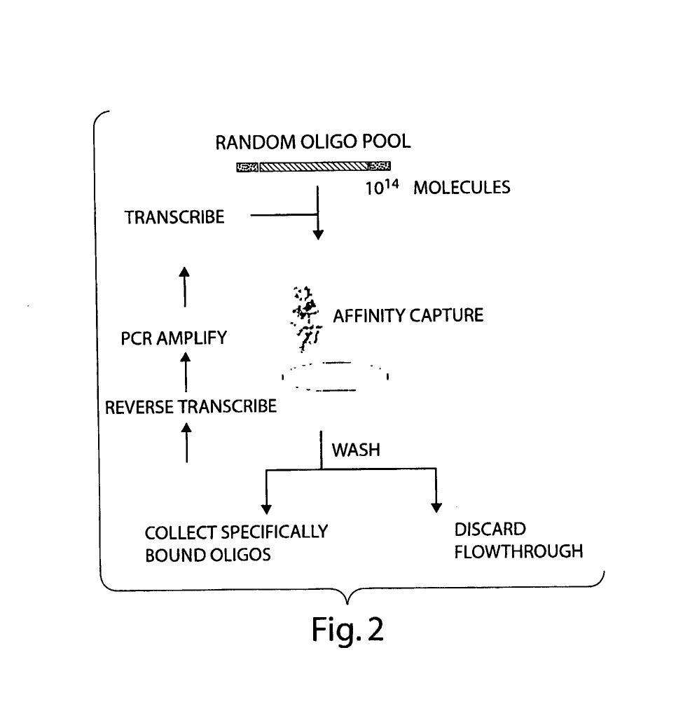

A suitable method for generating an aptamer is with the process entitled “Systematic Evolution of Ligands by Exponential Enrichment” (“SELEX™”) generally depicted for ribonucleic acid selections in FIG. 2 (For selections using a deoxyribonucleic acid pool, the reverse transcription and transcription steps depicted in FIG. 2 are omitted). The SELEX™ process is a method for the in vitro evolution of nucleic acid molecules with highly specific binding to target molecules and is described in, e.g., U.S. patent application Ser. No. 07/536,428, filed Jun. 11, 1990, now abandoned, U.S. Pat. No. 5,475,096 entitled “Nucleic Acid Ligands”, and U.S. Pat. No. 5,270,163 (see also WO 91/19813) entitled “Nucleic Acid Ligands”. Each SELEX™-identified nucleic acid ligand, i.e., each aptamer, is a specific ligand of a given target compound or molecule. The SELEX™ process is based on the unique insight that nucleic acids have sufficient capacity for forming a variety of two- and three-dimensional structures and sufficient chemical versatility available within their monomers to act as ligands (i.e., form specific binding pairs) with virtually any chemical compound, whether monomeric or polymeric. Molecules of any size or composition can serve as targets.

SELEX™ relies as a starting point upon a large library or pool of single stranded oligonucleotides comprising randomized sequences. The oligonucleotides can be modified or unmodified DNA, RNA, or DNA/RNA hybrids. In some examples, the pool comprises 100% random or partially random oligonucleotides. In other examples, the pool comprises random or partially random oligonucleotides containing at least one fixed sequence and/or conserved sequence incorporated within randomized sequence. In other examples, the pool comprises random or partially random oligonucleotides containing at least one fixed sequence and/or conserved sequence at its 5′ and/or 3′ end which may comprise a sequence shared by all the molecules of the oligonucleotide pool. Fixed sequences are sequences common to oligonucleotides in the pool which are incorporated for a preselected purpose such as, CpG motifs described further below, hybridization sites for PCR primers, promoter sequences for RNA polymerases (e.g., T3, T4, T7, and SP6), restriction sites, or homopolymeric sequences, such as poly A or poly T tracts, catalytic cores, sites for selective binding to affinity columns, and other sequences to facilitate cloning and/or sequencing of an oligonucleotide of interest. Conserved sequences are sequences, other than the previously described fixed sequences, shared by a number of aptamers that bind to the same target.

The oligonucleotides of the pool preferably include a randomized sequence portion as well as fixed sequences necessary for efficient amplification. Typically the oligonucleotides of the starting pool contain fixed 5′ and 3′ terminal sequences which flank an internal region of 30-50 random nucleotides. The randomized nucleotides can be produced in a number of ways including chemical synthesis and size selection from randomly cleaved cellular nucleic acids. Sequence variation in test nucleic acids can also be introduced or increased by mutagenesis before or during the selection/amplification iterations.

The random sequence portion of the oligonucleotide can be of any length and can comprise ribonucleotides and/or deoxyribonucleotides and can include modified or non-natural nucleotides or nucleotide analogs. See, e.g., U.S. Pat. No. 5,958,691; U.S. Pat. No. 5,660,985; U.S. Pat. No. 5,958,691; U.S. Pat. No. 5,698,687; U.S. Pat. No. 5,817,635; U.S. Pat. No. 5,672,695, and PCT Publication WO 92/07065. Random oligonucleotides can be synthesized from phosphodiester-linked nucleotides using solid phase oligonucleotide synthesis techniques well known in the art. See, e.g., Froehler et al., Nucl. Acid Res. 14: 5399-5467 (1986) and Froehler et al., Tet. Lett. 27:5575-5578 (1986). Random oligonucleotides can also be synthesized using solution phase methods such as triester synthesis methods. See, e.g., Sood et al., Nucl. Acid Res. 4:2557 (1977) and Hirose et al., Tet. Lett., 28:2449 (1978). Typical syntheses carried out on automated DNA synthesis equipment yield 10¹⁴-10¹⁶ individual molecules, a number sufficient for most SELEX™ experiments. Sufficiently large regions of random sequence in the sequence design increases the likelihood that each synthesized molecule is likely to represent a unique sequence.

The starting library of oligonucleotides may be generated by automated chemical synthesis on a DNA synthesizer. To synthesize randomized sequences, mixtures of all four nucleotides are added at each nucleotide addition step during the synthesis process, allowing for random incorporation of nucleotides. As stated above, in one embodiment, random oligonucleotides comprise entirely random sequences; however, in other embodiments, random oligonucleotides can comprise stretches of nonrandom or partially random sequences. Partially random sequences can be created by adding the four nucleotides in different molar ratios at each addition step.

The starting library of oligonucleotides may be, for example, RNA, DNA or RNA/DNA hybrid. In those instances where an RNA library is to be used as the starting library it is typically generated by transcribing a DNA library in vitro using T7 RNA polymerase or modified T7 RNA polymerases and purified. The RNA or DNA library is then mixed with the target under conditions favorable for binding and subjected to step-wise iterations of binding, partitioning and amplification, using the same general selection scheme, to achieve virtually any desired criterion of binding affinity and selectivity. More specifically, starting with a mixture containing the starting pool of nucleic acids, the SELEX™ method includes steps of: (a) contacting the mixture with the target under conditions favorable for binding; (b) partitioning unbound nucleic acids from those nucleic acids which have bound specifically to target molecules; (c) dissociating the nucleic acid-target complexes; (d) amplifying the nucleic acids dissociated from the nucleic acid-target complexes to yield a ligand-enriched mixture of nucleic acids; and (e) reiterating the steps of binding, partitioning, dissociating and amplifying through as many cycles as desired to yield highly specific, high affinity nucleic acid ligands to the target molecule. In those instances where RNA aptamers are being selected, the SELEX™ method further comprises the steps of: (i) reverse transcribing the nucleic acids dissociated from the nucleic acid-target complexes before amplification in step (d); and (ii) transcribing the amplified nucleic acids from step (d) before restarting the process.

Within a nucleic acid mixture containing a large number of possible sequences and structures, there is a wide range of binding affinities for a given target. A nucleic acid mixture comprising, for example, a 20 nucleotide randomized segment can have 4²⁰ candidate possibilities. Those which have the higher affinity constants for the target are most likely to bind to the target. After partitioning, dissociation and amplification, a second nucleic acid mixture is generated, enriched for the higher binding affinity candidates. Additional rounds of selection progressively favor the best ligands until the resulting nucleic acid mixture is predominantly composed of only one or a few sequences. These can then be cloned, sequenced and individually tested for binding affinity as pure ligands or aptamers.

Cycles of selection and amplification are repeated until a desired goal is achieved. In the most general case, selection/amplification is continued until no significant improvement in binding strength is achieved on repetition of the cycle. The method is typically used to sample approximately 10¹⁴ different nucleic acid species but may be used to sample as many as about 10¹⁸ different nucleic acid species. Generally, nucleic acid aptamer molecules are selected in a 5 to 20 cycle procedure. In one embodiment, heterogeneity is introduced only in the initial selection stages and does not occur throughout the replicating process.

In one embodiment of SELEX™, the selection process is so efficient at isolating those nucleic acid ligands that bind most strongly to the selected target, that only one cycle of selection and amplification is required. Such an efficient selection may occur, for example, in a chromatographic-type process wherein the ability of nucleic acids to associate with targets bound on a column operates in such a manner that the column is sufficiently able to allow separation and isolation of the highest affinity nucleic acid ligands.

In many cases, it is not necessarily desirable to perform the iterative steps of SELEX™ until a single nucleic acid ligand is identified. The target-specific nucleic acid ligand solution may include a family of nucleic acid structures or motifs that have a number of conserved sequences and a number of sequences which can be substituted or added without significantly affecting the affinity of the nucleic acid ligands to the target. By terminating the SELEX™ process prior to completion, it is possible to determine the sequence of a number of members of the nucleic acid ligand solution family.

A variety of nucleic acid primary, secondary and tertiary structures are known to exist. The structures or motifs that have been shown most commonly to be involved in non-Watson-Crick type interactions are referred to as hairpin loops, symmetric and asymmetric bulges, pseudoknots and myriad combinations of the same. Almost all known cases of such motifs suggest that they can be formed in a nucleic acid sequence of no more than 30 nucleotides. For this reason, it is often preferred that SELEX™ procedures with contiguous randomized segments be initiated with nucleic acid sequences containing a randomized segment of between about 20 to about 50 nucleotides and in some embodiments, about 30 to about 40 nucleotides. In one example, the 5′-fixed:random:3′-fixed sequence comprises a random sequence of about 30 to about 50 nucleotides.

The core SELEX™ method has been modified to achieve a number of specific objectives. For example, U.S. Pat. No. 5,707,796 describes the use of SELEX™ in conjunction with gel electrophoresis to select nucleic acid molecules with specific structural characteristics, such as bent DNA. U.S. Pat. No. 5,763,177 describes SELEX™ based methods for selecting nucleic acid ligands containing photo reactive groups capable of binding and/or photo-crosslinking to and/or photo-inactivating a target molecule. U.S. Pat. No. 5,567,588 and U.S. Pat. No. 5,861,254 describe SELEX™ based methods which achieve highly efficient partitioning between oligonucleotides having high and low affinity for a target molecule. U.S. Pat. No. 5,496,938 describes methods for obtaining improved nucleic acid ligands after the SELEX™ process has been performed. U.S. Pat. No. 5,705,337 describes methods for covalently linking a ligand to its target.

SELEX™ can also be used to obtain nucleic acid ligands that bind to more than one site on the target molecule, and to obtain nucleic acid ligands that include non-nucleic acid species that bind to specific sites on the target. SELEX™ provides means for isolating and identifying nucleic acid ligands which bind to any envisionable target, including large and small biomolecules such as nucleic acid-binding proteins and proteins not known to bind nucleic acids as part of their biological function as well as cofactors and other small molecules. For example, U.S. Pat. No. 5,580,737 discloses nucleic acid sequences identified through SELEX™ which are capable of binding with high affinity to caffeine and the closely related analog, theophylline.

Counter-SELEX™ is a method for improving the specificity of nucleic acid ligands to a target molecule by eliminating nucleic acid ligand sequences with cross-reactivity to one or more non-target molecules. Counter-SELEX™ is comprised of the steps of: (a) preparing a candidate mixture of nucleic acids; (b) contacting the candidate mixture with the target, wherein nucleic acids having an increased affinity to the target relative to the candidate mixture may be partitioned from the remainder of the candidate mixture; (c) partitioning the increased affinity nucleic acids from the remainder of the candidate mixture; (d) dissociating the increased affinity nucleic acids from the target; (e) contacting the increased affinity nucleic acids with one or more non-target molecules such that nucleic acid ligands with specific affinity for the non-target molecule(s) are removed; and (f) amplifying the nucleic acids with specific affinity only to the target molecule to yield a mixture of nucleic acids enriched for nucleic acid sequences with a relatively higher affinity and specificity for binding to the target molecule. As described above for SELEX™, cycles of selection and amplification are repeated as necessary until a desired goal is achieved.

One potential problem encountered in the use of nucleic acids as therapeutics and vaccines is that oligonucleotides in their phosphodiester form may be quickly degraded in body fluids by intracellular and extracellular enzymes such as endonucleases and exonucleases before the desired effect is manifest. The SELEX™ method thus encompasses the identification of high-affinity nucleic acid ligands containing modified nucleotides conferring improved characteristics on the ligand, such as improved in vivo stability or improved delivery characteristics. Examples of such modifications include chemical substitutions at the ribose and/or phosphate and/or base positions. SELEX™-identified nucleic acid ligands containing modified nucleotides are described, e.g., in U.S. Pat. No. 5,660,985, which describes oligonucleotides containing nucleotide derivatives chemically modified at the 2′ position of ribose, 5 position of pyrimidines, and 8 position of purines, U.S. Pat. No. 5,756,703 which describes oligonucleotides containing various 2′-modified pyrimidines, and U.S. Pat. No. 5,580,737 which describes highly specific nucleic acid ligands containing one or more nucleotides modified with 2′-amino (2′-NH₂), 2′-fluoro (2′-F), and/or 2′-O-methyl (2′-OMe) substituents.

Modifications of the nucleic acid ligands contemplated in this invention include, but are not limited to, those which provide other chemical groups that incorporate additional charge, polarizability, hydrophobicity, hydrogen bonding, electrostatic interaction, and fluxionality to the nucleic acid ligand bases or to the nucleic acid ligand as a whole. Modifications to generate oligonucleotide populations which are resistant to nucleases can also include one or more substitute internucleotide linkages, altered sugars, altered bases, or combinations thereof. Such modifications include, but are not limited to, 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, phosphorothioate or alkyl phosphate modifications, methylations, and unusual base-pairing combinations such as the isobases isocytidine and isoguanosine. Modifications can also include 3′ and 5′ modifications such as capping.

In one embodiment, oligonucleotides are provided in which the P(O)O group is replaced by P(O)S (“thioate”), P(S)S (“dithioate”), P(O)NR₂ (“amidate”), P(O)R, P(O)OR′, CO or CH₂ (“formacetal”) or 3′-amine (—NH—CH₂—CH₂—), wherein each R or R′ is independently H or substituted or unsubstituted alkyl. Linkage groups can be attached to adjacent nucleotides through an —O—, —N—, or —S— linkage. Not all linkages in the oligonucleotide are required to be identical.

In further embodiments, the oligonucleotides comprise modified sugar groups, for example, one or more of the hydroxyl groups is replaced with halogen, aliphatic groups, or functionalized as ethers or amines. In one embodiment, the 2′-position of the furanose residue is substituted by any of an O-methyl, O-alkyl, O-allyl, S-alkyl, S-allyl, or halo group. Methods of synthesis of 2′-modified sugars are described, e.g., in Sproat, et al., Nucl. Acid Res. 19:733-738 (1991); Cotten, et al., Nucl. Acid Res. 19:2629-2635 (1991); and Hobbs, et al., Biochemistry 12:5138-5145 (1973). Other modifications are known to one of ordinary skill in the art. Such modifications may be pre-SELEX™ process modifications or post-SELEX™ process modifications (modification of previously identified unmodified ligands) or may be made by incorporation into the SELEX™ process.

Pre-SELEX™ process modifications or those made by incorporation into the SELEX™ process yield nucleic acid ligands with both specificity for their SELEX™ target and improved stability, e.g., in vivo stability. Post-SELEX™ process modifications made to nucleic acid ligands may result in improved stability, e.g., in vivo stability without adversely affecting the binding capacity of the nucleic acid ligand.

The SELEX™ method encompasses combining selected oligonucleotides with other selected oligonucleotides and non-oligonucleotide functional units as described in U.S. Pat. No. 5,637,459 and U.S. Pat. No. 5,683,867. The SELEX™ method further encompasses combining selected nucleic acid ligands with lipophilic or non-immunogenic high molecular weight compounds in a diagnostic or therapeutic complex, as described, e.g., in U.S. Pat. No. 6,011,020, U.S. Pat. No. 6,051,698, and PCT Publication No. WO 98/18480. These patents and applications teach the combination of a broad array of shapes and other properties, with the efficient amplification and replication properties of oligonucleotides, and with the desirable properties of other molecules.

The identification of nucleic acid ligands to small, flexible peptides via the SELEX™ method has also been explored. Small peptides have flexible structures and usually exist in solution in an equilibrium of multiple conformers, and thus it was initially thought that binding affinities may be limited by the conformational entropy lost upon binding a flexible peptide. However, the feasibility of identifying nucleic acid ligands to small peptides in solution was demonstrated in U.S. Pat. No. 5,648,214. In this patent, high affinity RNA nucleic acid ligands to substance P, an 11 amino acid peptide, were identified.

The aptamers with specificity and binding affinity to the target(s) of the present invention are typically selected by the SELEX™ process as described herein. As part of the SELEX™ process, the sequences selected to bind to the target are then optionally minimized to determine the minimal sequence having the desired binding affinity. The selected sequences and/or the minimized sequences are optionally modified by performing random or directed mutagenesis of the sequence to increase binding affinity or alternatively to determine which positions in the sequence are essential for binding activity. Additionally, selections can be performed with sequences incorporating modified nucleotides to stabilize the aptamer molecules against degradation in vivo.

### 2′ Modified SELEX™

In order for an aptamer to be suitable for use as a therapeutic, it is preferably inexpensive to synthesize, safe and stable in vivo. Wild-type RNA and DNA aptamers are typically not stable in vivo because of their susceptibility to degradation by nucleases. Resistance to nuclease degradation can be greatly increased by the incorporation of modifying groups at the 2′-position.

Fluoro and amino groups have been successfully incorporated into oligonucleotide pools from which aptamers have been subsequently selected. However, these modifications greatly increase the cost of synthesis of the resultant aptamer, and may introduce safety concerns in some cases because of the possibility that the modified nucleotides could be recycled into host DNA by degradation of the modified oligonucleotides and subsequent use of the nucleotides as substrates for DNA synthesis.

Aptamers that contain 2′-O-methyl (“2′-OMe”) nucleotides, as provided herein, overcome many of these drawbacks. Oligonucleotides containing 2′-OMe nucleotides are nuclease-resistant and inexpensive to synthesize. Although 2′-OMe nucleotides are ubiquitous in biological systems, natural polymerases do not accept 2′-OMe NTPs as substrates under physiological conditions, thus there are no safety concerns over the recycling of 2′-OMe nucleotides into host DNA. The SELEX™ method used to generate 2′-modified aptamers is described, e.g., in U.S. Provisional Patent Application Ser. No. 60/430,761, filed Dec. 3, 2002, U.S. Provisional Patent Application Ser. No. 60/487,474, filed Jul. 15, 2003, U.S. Provisional Patent Application Ser. No. 60/517,039, filed Nov. 4, 2003, U.S. patent application Ser. No. 10/729,581, filed Dec. 3, 2003, and U.S. patent application Ser. No. 10/873,856, filed Jun. 21, 2004, entitled “Method for in vitro Selection of 2′-O-methyl Substituted Nucleic Acids”, each of which is herein incorporated by reference in its entirety.

The present invention includes aptamers that bind to and modulate the function of IgE which contain modified nucleotides (e.g., nucleotides which have a modification at the 2′ position) to make the oligonucleotide more stable than the unmodified oligonucleotide to enzymatic and chemical degradation as well as thermal and physical degradation. Although there are several examples of 2′-OMe containing aptamers in the literature (see, e.g., Green et al., Current Biology 2, 683-695, 1995) these were generated by the in vitro selection of libraries of modified transcripts in which the C and U residues were 2′-fluoro (2′-F) substituted and the A and G residues were 2′-OH. Once functional sequences were identified then each A and G residue was tested for tolerance to 2′-OMe substitution, and the aptamer was re-synthesized having all A and G residues which tolerated 2′-OMe substitution as 2′-OMe residues. Most of the A and G residues of aptamers generated in this two-step fashion tolerate substitution with 2′-OMe residues, although, on average, approximately 20% do not. Consequently, aptamers generated using this method tend to contain from two to four 2′-OH residues, and stability and cost of synthesis are compromised as a result. By incorporating modified nucleotides into the transcription reaction which generate stabilized oligonucleotides used in oligonucleotide pools from which aptamers are selected and enriched by SELEX™ (and/or any of its variations and improvements, including those described herein), the methods of the present invention eliminate the need for stabilizing the selected aptamer oligonucleotides (e.g., by resynthesizing the aptamer oligonucleotides with modified nucleotides).

In one embodiment, the present invention provides aptamers comprising combinations of 2′-OH, 2′-F, 2′-deoxy, and 2′-OMe modifications of the ATP, GTP, CTP, TTP, and UTP nucleotides. In another embodiment, the present invention provides aptamers comprising combinations of 2′-OH, 2′-F, 2′-deoxy, 2′-OMe, 2′—NH₂, and 2′-methoxyethyl modifications of the ATP, GTP, CTP, TTP, and UTP nucleotides. In another embodiment, the present invention provides aptamers comprising 5⁶ combinations of 2′-OH, 2′-F, 2′-deoxy, 2′-OMe, 2′—NH₂, and 2′-methoxyethyl modifications of the ATP, GTP, CTP, TTP, and UTP nucleotides.

2′ modified aptamers of the invention are created using modified polymerases, e.g., a modified T7 polymerase, having a rate of incorporation of modified nucleotides having bulky substituents at the furanose 2′ position that is higher than that of wild-type polymerases. For example, a mutant T7 polymerase (Y639F) in which the tyrosine residue at position 639 has been changed to phenylalanine readily utilizes 2′deoxy, 2′amino-, and 2′fluoro-nucleotide triphosphates (NTPs) as substrates and has been widely used to synthesize modified RNAs for a variety of applications. However, this mutant T7 polymerase reportedly can not readily utilize (i.e., incorporate) NTPs with bulky 2′-substituents such as 2′-OMe or 2′-azido (2′-N₃) substituents. For incorporation of bulky 2′ substituents, a T7 polymerase mutant (Y639F/H784A) having the histidine at position 784 changed to an alanine residue in addition to the Y639F mutation has been described and has been used in limited circumstances to incorporate modified pyrimidine NTPs. See Padilla, R. and Sousa, R., Nucleic Acids Res., 2002, 30(24): 138. A mutant T7 polymerase (H784A) having the histidine at position 784 changed to an alanine residue has also been described. Padilla et al., Nucleic Acids Research, 2002, 30: 138. In both the Y639F/H784A and H784A mutant T7 polymerases, the change to a smaller amino acid residue such as alanine allows for the incorporation of bulkier nucleotide substrates, e.g., 2′-OMe substituted nucleotides.

Generally, it has been found that under the conditions disclosed herein, the Y693F mutant can be used for the incorporation of all 2′-OMe substituted NTPs except GTP and the Y639F/H784A mutant can be used for the incorporation of all 2′-OMe substituted NTPs including GTP. It is expected that the H784A mutant possesses properties similar to the Y639F and the Y639F/H784A mutants when used under the conditions disclosed herein.

2′-modified oligonucleotides may be synthesized entirely of modified nucleotides, or with a subset of modified nucleotides. The modifications can be the same or different. All nucleotides may be modified, and all may contain the same modification. All nucleotides may be modified, but contain different modifications, e.g., all nucleotides containing the same base may have one type of modification, while nucleotides containing other bases may have different types of modification. All purine nucleotides may have one type of modification (or are unmodified), while all pyrimidine nucleotides have another, different type of modification (or are unmodified). In this way, transcripts, or pools of transcripts are generated using any combination of modifications, including for example, ribonucleotides (2′-OH), deoxyribonucleotides (2′-deoxy), 2′-F, and 2′-OMe nucleotides. A transcription mixture containing 2′-OMe C and U and 2′-OH A and G is referred to as an “rRmY” mixture and aptamers selected therefrom are referred to as “rRmY” aptamers. A transcription mixture containing deoxy A and G and 2′-OMe U and C is referred to as a “dRmY” mixture and aptamers selected therefrom are referred to as “dRmY” aptamers. A transcription mixture containing 2′-OMe A, C, and U, and 2′-OH G is referred to as a “rGmH” mixture and aptamers selected therefrom are referred to as “rGmH” aptamers. A transcription mixture alternately containing 2′-OMe A, C, U and G and 2′-OMe A, U and C and 2′-F G is referred to as an “alternating mixture” and aptamers selected therefrom are referred to as “alternating mixture” aptamers. A transcription mixture containing 2′-OMe A, U, C, and G, where up to 10% of the G's are ribonucleotides is referred to as a “r/mGmH” mixture and aptamers selected therefrom are referred to as “r/mGmH” aptamers. A transcription mixture containing 2′-OMe A, U, and C, and 2′-F G is referred to as a “fGmH” mixture and aptamers selected therefrom are referred to as “fGmH” aptamers. A transcription mixture containing 2′-OMe A, U, and C, and deoxy G is referred to as a “dGmH” mixture and aptamers selected therefrom are referred to as “dGmH” aptamers. A transcription mixture containing deoxy A, and 2′-OMe C, G and U is referred to as a “dAmB” mixture and aptamers selected therefrom are referred to as “dAmB” aptamers, and a transcription mixture containing all 2′-OH nucleotides is referred to as a “rN” mixture and aptamers selected therefrom are referred to as “rN” or “rRrY” aptamers. A “mRmY” aptamer is one containing all 2′-O-methyl nucleotides and is usually derived from a r/mGmH oligonucleotide by post-SELEX™ replacement, when possible, of any 2′-OH Gs with 2′-OMe Gs.

A preferred embodiment includes any combination of 2′-OH, 2′-deoxy and 2′-OMe nucleotides. A more preferred embodiment includes any combination of 2′-deoxy and 2′-OMe nucleotides. An even more preferred embodiment is with any combination of 2′-deoxy and 2′-OMe nucleotides in which the pyrimidines are 2′-OMe (such as dRmY, mRmY or dGmH).

Incorporation of modified nucleotides into the aptamers of the invention is accomplished before (pre-) the selection process (e.g., a pre-SELEX™ process modification). Optionally, aptamers of the invention in which modified nucleotides have been incorporated by pre-SELEX™ process modification can be further modified by post-SELEX™ process modification (i.e., a post-SELEX™ process modification after a pre-SELEX™ modification). Pre-SELEX™ process modifications yield modified nucleic acid ligands with specificity for the SELEX™ target and also improved in vivo stability. Post-SELEX™ process modifications, i.e., modification (e.g., truncation, deletion, substitution or additional nucleotide modifications of previously identified ligands having nucleotides incorporated by pre-SELEX™ process modification) can result in a further improvement of in vivo stability without adversely affecting the binding capacity of the nucleic acid ligand having nucleotides incorporated by pre-SELEX™ process modification.

To generate pools of 2′-modified (e.g., 2′-OMe) RNA transcripts in conditions under which a polymerase accepts 2′-modified NTPs the preferred polymerase is the Y693F/H784A mutant or the Y693F mutant. Other polymerases, particularly those that exhibit a high tolerance for bulky 2′-substituents, may also be used in the present invention. Such polymerases can be screened for this capability by assaying their ability to incorporate modified nucleotides under the transcription conditions disclosed herein.

A number of factors have been determined to be important for the transcription conditions useful in the methods disclosed herein. For example, increases in the yields of modified transcript are observed when a leader sequence is incorporated into the 5′ end of a fixed sequence at the 5′ end of the DNA transcription template, such that at least about the first 6 residues of the resultant transcript are all purines.

Another important factor in obtaining transcripts incorporating modified nucleotides is the presence or concentration of 2′-OH GTP. Transcription can be divided into two phases: the first phase is initiation, during which an NTP is added to the 3′-hydroxyl end of GTP (or another substituted guanosine) to yield a dinucleotide which is then extended by about 10-12 nucleotides; the second phase is elongation, during which transcription proceeds beyond the addition of the first about 10-12 nucleotides. It has been found that small amounts of 2′-OH GTP added to a transcription mixture containing an excess of 2′-OMe GTP are sufficient to enable the polymerase to initiate transcription using 2′-OH GTP, but once transcription enters the elongation phase the reduced discrimination between 2′-OMe and 2′-OH GTP, and the excess of 2′-OMe GTP over 2′-OH GTP allows the incorporation of principally the 2′-OMe GTP.

Another important factor in the incorporation of 2′-OMe substituted nucleotides into transcripts is the use of both divalent magnesium and manganese in the transcription mixture. Different combinations of concentrations of magnesium chloride and manganese chloride have been found to affect yields of 2′-O-methylated transcripts, the optimum concentration of the magnesium and manganese chloride being dependent on the concentration in the transcription reaction mixture of NTPs which complex divalent metal ions. To obtain the greatest yields of maximally 2′ substituted O-methylated transcripts (i.e., all A, C, and U and about 90% of G nucleotides), concentrations of approximately 5 mM magnesium chloride and 1.5 mM manganese chloride are preferred when each NTP is present at a concentration of 0.5 mM. When the concentration of each NTP is 1.0 mM, concentrations of approximately 6.5 mM magnesium chloride and 2.0 mM manganese chloride are preferred. When the concentration of each NTP is 2.0 mM, concentrations of approximately 9.6 mM magnesium chloride and 2.9 mM manganese chloride are preferred. In any case, departures from these concentrations of up to two-fold still give significant amounts of modified transcripts.

Priming transcription with GMP or guanosine is also important. This effect results from the specificity of the polymerase for the initiating nucleotide. As a result, the 5′-terminal nucleotide of any transcript generated in this fashion is likely to be 2′-OH G. The preferred concentration of GMP (or guanosine) is 0.5 mM and even more preferably 1 mM. It has also been found that including PEG, preferably PEG-8000, in the transcription reaction is useful to maximize incorporation of modified nucleotides.

For maximum incorporation of 2′-OMe ATP (100%), UTP (100%), CTP (100%) and GTP (˜90%) (“r/mGmH”) into transcripts the following conditions are preferred: HEPES buffer 200 mM, DTT 40 mM, spermidine 2 mM, PEG-8000 10% (w/v), Triton X-100 0.01% (w/v), MgCl₂ 5 mM (6.5 mM where the concentration of each 2′-OMe NTP is 1.0 mM), MnCl₂ 1.5 mM (2.0 mM where the concentration of each 2′-OMe NTP is 1.0 mM), 2′-OMe NTP (each) 500 μM (more preferably, 1.0 mM), 2′-OH GTP 30 μM, 2′-OH GMP 500 μM, pH 7.5, Y639F/H784A T7 RNA Polymerase 15 units/mL, inorganic pyrophosphatase 5 units/mL, and an all-purine leader sequence of at least 8 nucleotides long. As used herein, one unit of the Y639F/H784A mutant T7 RNA polymerase (or any other mutant T7 RNA polymerase specified herein) is defined as the amount of enzyme required to incorporate 1 nmole of 2′-OMe NTPs into transcripts under the r/mGmH conditions. As used herein, one unit of inorganic pyrophosphatase is defined as the amount of enzyme that will liberate 1.0 mole of inorganic orthophosphate per minute at pH 7.2 and 25° C.

For maximum incorporation (100%) of 2′-OMe ATP, UTP and CTP (“rGmH”) into transcripts the following conditions are preferred: HEPES buffer 200 mM, DTT 40 mM, spermidine 2 mM, PEG-8000 10% (w/v), Triton X-100 0.01% (w/v), MgCl₂ 5 mM (9.6 mM where the concentration of each 2′-OMe NTP is 2.0 mM), MnCl₂ 1.5 mM (2.9 mM where the concentration of each 2′-OMe NTP is 2.0 mM), 2′-OMe NTP (each) 500 μM (more preferably, 2.0 mM), pH 7.5, Y639F T7 RNA Polymerase 15 units/ml, inorganic pyrophosphatase 5 units/mL, and an all-purine leader sequence of at least 8 nucleotides long.

For maximum incorporation (100%) of 2′-OMe UTP and CTP (“rRmY”) into transcripts the following conditions are preferred: HEPES buffer 200 mM, DTT 40 mM, spermidine 2 mM, PEG-8000 10% (w/v), Triton X-100 0.01% (w/v), MgCl₂ 5 mM (9.6 mM where the concentration of each 2′-OMe NTP is 2.0 mM), MnCl₂ 1.5 mM (2.9 mM where the concentration of each 2′-OMe NTP is 2.0 mM), 2′-OMe NTP (each) 500 μM (more preferably, 2.0 mM), pH 7.5, Y639F/H784A T7 RNA Polymerase 15 units/mL, inorganic pyrophosphatase 5 units/mL, and an all-purine leader sequence of at least 8 nucleotides long.

For maximum incorporation (100%) of deoxy ATP and GTP and 2′-OMe UTP and CTP (“dRmY”) into transcripts the following conditions are preferred: HEPES buffer 200 mM, DTT 40 mM, spermine 2 mM, spermidine 2 mM, PEG-8000 10% (w/v), Triton X-100 0.01% (w/v), MgCl₂ 9.6 mM, MnCl₂ 2.9 mM, 2′-OMe NTP (each) 2.0 mM, pH 7.5, Y639F T7 RNA Polymerase 15 units/mL, inorganic pyrophosphatase 5 units/mL, and an all-purine leader sequence of at least 8 nucleotides long.

For maximum incorporation (100%) of 2′-OMe ATP, UTP and CTP and 2′-F GTP (“fGmH”) into transcripts the following conditions are preferred: HEPES buffer 200 mM, DTT 40 mM, spermidine 2 mM, PEG-8000 10% (w/v), Triton X-100 0.01% (w/v), MgCl₂ 9.6 mM, MnCl₂ 2.9 mM, 2′-OMe NTP (each) 2.0 mM, pH 7.5, Y639F T7 RNA Polymerase 15 units/mL, inorganic pyrophosphatase 5 units/mL, and an all-purine leader sequence of at least 8 nucleotides long.

For maximum incorporation (100%) of deoxy ATP and 2′-OMe UTP, GTP and CTP (“dAmB”) into transcripts the following conditions are preferred: HEPES buffer 200 mM, DTT 40 mM, spermidine 2 mM, PEG-8000 100% (w/v), Triton X-100 0.01% (w/v), MgCl₂ 9.6 mM, MnCl₂ 2.9 mM, 2′-OMe NTP (each) 2.0 mM, pH 7.5, Y639F T17 RNA Polymerase 15 units/mL, inorganic pyrophosphatase 5 units/mL, and an all-purine leader sequence of at least 8 nucleotides long.

For each of the above (a) transcription is preferably performed at a temperature of from about 20° C. to about 50° C., preferably from about 30° C. to 45° C., and more preferably at about 37° C. for a period of at least two hours and (b) 50-300 nM of a double stranded DNA transcription template is used (200 nM template is used in round I to increase diversity (300 nM template is used in dRmY transcriptions)), and for subsequent rounds approximately 50 nM, a 1/10 dilution of an optimized PCR reaction, using conditions described herein, is used). The preferred DNA transcription templates are described below (where ARC254 and ARC256 transcribe under all 2′-OMe conditions and ARC255 transcribes under rRmY conditions).

ARC254

SEQ ID NO 1

5′-CATCGATGCTAGTCGTAACGATCCNNNNNNNNNNNNNNNNNNNNNNN
NNNNNNNNCGAGAACGTTCTCTCCTCTCCCTATAGTGAGTCGTATTA-3′
ARC255

SEQ ID NO 2

5′-CATGCATCGCGACTGACTAGCCGNNNNNNNNNNNNNNNNNNNNNNNN
NNNNNNGTAGAACGTTCTCTCCTCTCCCTATAGTGACTCGTATTA-3′
ARC256

SEQ ID NO 3

5′-CATCGATCGATCGATCGACAGCGNNNNNNNNNNNNNNNNNNNNNNNN
NNNNNNGTAGAACGTTCTCTCCTCTCCCTATAGTGAGTCGTATTA-3′

Under rN transcription conditions of the present invention, the transcription reaction mixture comprises 2′-OH adenosine triphosphates (ATP), 2′-OH guanosine triphosphates (GTP), 2′-OH cytidine triphosphates (CTP), and 2′-OH uridine triphosphates (UTP). The modified oligonucleotides produced using the rN transcription mixtures of the present invention comprise substantially all 2′-OH adenosine, 2′-OH guanosine, 2′-OH cytidine, and 2′-OH uridine. In a preferred embodiment of rN transcription, the resulting modified oligonucleotides comprise a sequence where at least 80% of all adenosine nucleotides are 2′-OH adenosine, at least 80% of all guanosine nucleotides are 2′-OH guanosine, at least 80% of all cytidine nucleotides are 2′-OH cytidine, and at least 80% of all uridine nucleotides are 2′-OH uridine. In a more preferred embodiment of rN transcription, the resulting modified oligonucleotides of the present invention comprise a sequence where at least 90% of all adenosine nucleotides are 2′-OH adenosine, at least 90% of all guanosine nucleotides are 2′-OH guanosine, at least 90% of all cytidine nucleotides are 2′-OH cytidine, and at least 90% of all uridine nucleotides are 2′-OH uridine. In a most preferred embodiment of rN transcription, the modified oligonucleotides of the present invention comprise a sequence where 100% of all adenosine nucleotides are 2′-OH adenosine, 100% of all guanosine nucleotides are 2′-OH guanosine, 100% of all cytidine nucleotides are 2′-OH cytidine, and 100% of all uridine nucleotides are 2′-OH uridine.

Under rRmY transcription conditions of the present invention, the transcription reaction mixture comprises 2′-OH adenosine triphosphates, 2′-OH guanosine triphosphates, 2′-O-methyl cytidine triphosphates, and 2′-O-methyl uridine triphosphates. The modified oligonucleotides produced using the rRmY transcription mixtures of the present invention comprise substantially all 2′-OH adenosine, 2′-OH guanosine, 2′-O-methyl cytidine and 2′-O-methyl uridine. In a preferred embodiment, the resulting modified oligonucleotides comprise a sequence where at least 80% of all adenosine nucleotides are 2′-OH adenosine, at least 80% of all guanosine nucleotides are 2′-OH guanosine, at least 80% of all cytidine nucleotides are 2′-O-methyl cytidine and at least 80% of all uridine nucleotides are 2′-O-methyl uridine. In a more preferred embodiment, the resulting modified oligonucleotides comprise a sequence where at least 90% of all adenosine nucleotides are 2′-OH adenosine, at least 90% of all guanosine nucleotides are 2′-OH guanosine, at least 90% of all cytidine nucleotides are 2′-O-methyl cytidine and at least 90% of all uridine nucleotides are 2′-O-methyl uridine In a most preferred embodiment, the resulting modified oligonucleotides comprise a sequence where 100% of all adenosine nucleotides are 2′-OH adenosine, 100% of all guanosine nucleotides are 2′-OH guanosine, 100% of all cytidine nucleotides are 2′-O-methyl cytidine and 100% of all uridine nucleotides are 2′-O-methyl uridine.

Under dRmY transcription conditions of the present invention, the transcription reaction mixture comprises 2′-deoxy adenosine triphosphates, 2′-deoxy guanosine triphosphates, 2′-O-methyl cytidine triphosphates, and 2′-O-methyl uridine triphosphates. The modified oligonucleotides produced using the dRmY transcription conditions of the present invention comprise substantially all 2′-deoxy adenosine, 2′-deoxy guanosine, 2′-O-methyl cytidine, and 2′-O-methyl uridine. In a preferred embodiment, the resulting modified oligonucleotides of the present invention comprise a sequence where at least 80% of all adenosine nucleotides are 2′-deoxy adenosine, at least 80% of all guanosine nucleotides are 2′-deoxy guanosine, at least 80% of all cytidine nucleotides are 2′-O-methyl cytidine, and at least 80% of all uridine nucleotides are 2′-O-methyl uridine. In a more preferred embodiment, the resulting modified oligonucleotides of the present invention comprise a sequence where at least 90% of all adenosine nucleotides are 2′-deoxy adenosine, at least 90% of all guanosine nucleotides are 2′-deoxy guanosine, at least 90% of all cytidine nucleotides are 2′-O-methyl cytidine, and at least 90% of all uridine nucleotides are 2′-O-methyl uridine. In a most preferred embodiment, the resulting modified oligonucleotides of the present invention comprise a sequence where 100% of all adenosine nucleotides are 2-deoxy adenosine, 100% of all guanosine nucleotides are 2′-deoxy guanosine, 100% of all cytidine nucleotides are 2′-O-methyl cytidine, and 100% of all uridine nucleotides are 2′-O-methyl uridine.

Under rGmH transcription conditions of the present invention, the transcription reaction mixture comprises 2′-OH guanosine triphosphates, 2′-O-methyl cytidine triphosphates, 2′-O-methyl uridine triphosphates, and 2′-O-methyl adenosine triphosphates. The modified oligonucleotides produced using the rGmH transcription mixtures of the present invention comprise substantially all 2′-OH guanosine, 2′-O-methyl cytidine, 2′-O-methyl uridine, and 2′-O-methyl adenosine. In a preferred embodiment, the resulting modified oligonucleotides comprise a sequence where at least 80% of all guanosine nucleotides are 2′-OH guanosine, at least 80% of all cytidine nucleotides are 2′-O-methyl cytidine, at least 80% of all uridine nucleotides are 2′-O-methyl uridine, and at least 80% of all adenosine nucleotides are 2′-O-methyl adenosine. In a more preferred embodiment, the resulting modified oligonucleotides comprise a sequence where at least 90% of all guanosine nucleotides are 2′-OH guanosine, at least 90% of all cytidine nucleotides are 2′-O-methyl cytidine, at least 90% of all uridine nucleotides are 2′-O-methyl uridine, and at least 90% of all adenosine nucleotides are 2′-O-methyl adenosine. In a most preferred embodiment, the resulting modified oligonucleotides comprise a sequence where 100% of all guanosine nucleotides are 2′-OH guanosine, 100% of all cytidine nucleotides are 2′-O-methyl cytidine, 100% of all uridine nucleotides are 2′-O-methyl uridine, and 100% of all adenosine nucleotides are 2′-O-methyl adenosine.

Under r/mGmH transcription conditions of the present invention, the transcription reaction mixture comprises 2′-O-methyl adenosine triphosphate, 2′-O-methyl cytidine triphosphate, 2′-O-methyl guanosine triphosphate, 2′-O-methyl uridine triphosphate and 2′-OH guanosine triphosphate. The resulting modified oligonucleotides produced using the r/mGmH transcription mixtures of the present invention comprise substantially all 2′-O-methyl adenosine, 2′-O-methyl cytidine, 2′-O-methyl guanosine, and 2′-O-methyl uridine, wherein the population of guanosine nucleotides has a maximum of about 10% 2′-OH guanosine. In a preferred embodiment, the resulting r/mGmH modified oligonucleotides of the present invention comprise a sequence where at least 80% of all adenosine nucleotides are 2′-O-methyl adenosine, at least 80% of all cytidine nucleotides are 2′-O-methyl cytidine, at least 80% of all guanosine nucleotides are 2′-O-methyl guanosine, at least 80% of all uridine nucleotides are 2′-O-methyl uridine, and no more than about 10% of all guanosine nucleotides are 2′-OH guanosine. In a more preferred embodiment, the resulting modified oligonucleotides comprise a sequence where at least 90% of all adenosine nucleotides are 2′-O-methyl adenosine, at least 90% of all cytidine nucleotides are 2′-O-methyl cytidine, at least 90% of all guanosine nucleotides are 2′-O-methyl guanosine, at least 90% of all uridine nucleotides are 2′-O-methyl uridine, and no more than about 10% of all guanosine nucleotides are 2′-OH guanosine. In a most preferred embodiment, the resulting modified oligonucleotides comprise a sequence where 100% of all adenosine nucleotides are 2′-O-methyl adenosine, 100% of all cytidine nucleotides are 2′-O-methyl cytidine, 90% of all guanosine nucleotides are 2′-O-methyl guanosine, and 100% of all uridine nucleotides are 2′-O-methyl uridine, and no more than about 10% of all guanosine nucleotides are 2′-OH guanosine.

Under fGmH transcription conditions of the present invention, the transcription reaction mixture comprises 2′-O-methyl adenosine triphosphates, 2′-O-methyl uridine triphosphates, 2′-O-methyl cytidine triphosphates, and 2′-F guanosine triphosphates. The modified oligonucleotides produced using the fGmH transcription conditions of the present invention comprise substantially all 2′-O-methyl adenosine, 2′-O-methyl uridine, 2′-O-methyl cytidine, and 2′-F guanosine. In a preferred embodiment, the resulting modified oligonucleotides comprise a sequence where at least 80% of all adenosine nucleotides are 2′-O-methyl adenosine, at least 80% of all uridine nucleotides are 2′-O-methyl uridine, at least 80% of all cytidine nucleotides are 2′-O-methyl cytidine, and at least 80% of all guanosine nucleotides are 2′-F guanosine. In a more preferred embodiment, the resulting modified oligonucleotides comprise a sequence where at least 90% of all adenosine nucleotides are 2′-O-methyl adenosine, at least 90% of all uridine nucleotides are 2′-O-methyl uridine, at least 90% of all cytidine nucleotides are 2′-O-methyl cytidine, and at least 90% of all guanosine nucleotides are 2′-F guanosine. In a most preferred embodiment, the resulting modified oligonucleotides comprise a sequence where 100% of all adenosine nucleotides are 2′-O-methyl adenosine, 100% of all uridine nucleotides are 2′-O-methyl uridine, 100% of all cytidine nucleotides are 2′-O-methyl cytidine, and 100% of all guanosine nucleotides are 2′-F guanosine.

Under dAmB transcription conditions of the present invention, the transcription reaction mixture comprises 2′-deoxy adenosine triphosphates, 2′-O-methyl cytidine triphosphates, 2′-O-methyl guanosine triphosphates, and 2′-O-methyl uridine triphosphates. The modified oligonucleotides produced using the dAmB transcription mixtures of the present invention comprise substantially all 2′-deoxy adenosine, 2′-O-methyl cytidine, 2′-O-methyl guanosine, and 2′-O-methyl uridine. In a preferred embodiment, the resulting modified oligonucleotides comprise a sequence where at least 80% of all adenosine nucleotides are 2′-deoxy adenosine, at least 80% of all cytidine nucleotides are 2′-O-methyl cytidine, at least 80% of all guanosine nucleotides are 2′-O-methyl guanosine, and at least 80% of all uridine nucleotides are 2′-O-methyl uridine. In a more preferred embodiment, the resulting modified oligonucleotides comprise a sequence where at least 90% of all adenosine nucleotides are 2′-deoxy adenosine, at least 90% of all cytidine nucleotides are 2′-O-methyl cytidine, at least 90% of all guanosine nucleotides are 2′-O-methyl guanosine, and at least 90% of all uridine nucleotides are 2′-O-methyl uridine. In a most preferred embodiment, the resulting modified oligonucleotides of the present invention comprise a sequence where 100% of all adenosine nucleotides are 2′-deoxy adenosine, 100% of all cytidine nucleotides are 2′-O-methyl cytidine, 100% of all guanosine nucleotides are 2′-O-methyl guanosine, and 100% of all uridine nucleotides are 2′-O-methyl uridine.

In each case, the transcription products can then be used as the library in the SELEX™ process to identify aptamers and/or to determine a conserved motif of sequences that have binding specificity to a given target. The resulting sequences are already partially stabilized, eliminating this step from the process to arrive at an modified aptamer sequence and giving a more highly stabilized aptamer as a result. Another advantage of the 2′-OMe SELEX™ process is that the resulting sequences are likely to have fewer 2′-OH nucleotides required in the sequence, possibly none. To the extent 2′OH nucleotides remain they can be removed by performing post-SELEX™ modifications.

As described below, lower but still useful yields of transcripts fully incorporating 2′ substituted nucleotides can be obtained under conditions other than the optimized conditions described above. For example, variations to the above transcription conditions include:

The HEPES buffer concentration can range from 0 to 1 M. The present invention also contemplates the use of other buffering agents having a pKa between 5 and 10 including, for example, Tris-hydroxymethyl-aminomethane.

The DTT concentration can range from 0 to 400 mM. The methods of the present invention also provide for the use of other reducing agents including, for example, mercaptoethanol.

The spermidine and/or spermine concentration can range from 0 to 20 mM.

The PEG-8000 concentration can range from 0 to 50% (w/v). The methods of the present invention also provide for the use of other hydrophilic polymer including, for example, other molecular weight PEG or other polyalkylene glycols.

The Triton X-100 concentration can range from 0 to 0.1% (w/v). The methods of the present invention also provide for the use of other non-ionic detergents including, for example, other detergents, including other Triton-X detergents.

The MgCl₂ concentration can range from 0.5 mM to 50 mM. The MnCl₂ concentration can range from 0.15 mM to 15 mM. Both MgCl₂ and MnCl₂ must be present within the ranges described and in a preferred embodiment are present in about a 10 to about 3 ratio of MgCl₂:MnCl₂, preferably, the ratio is about 3-5:1, more preferably, the ratio is about 3-4:1.

The 2′-OMe NTP concentration (each NTP) can range from 5 μM to 5 mM.

The 2′-OH GTP concentration can range from 0 μM to 300 μM.

The 2′-OH GMP concentration can range from 0 to 5 mM.

The pH can range from pH 6 to pH 9. The methods of the present invention can be practiced within the pH range of activity of most polymerases that incorporate modified nucleotides. In addition, the methods of the present invention provide for the optional use of chelating agents in the transcription reaction condition including, for example, EDTA, EGTA, and DTT.

### Aptamer Medicinal Chemistry

Aptamer Medicinal Chemistry is an aptamer improvement technique in which sets of variant aptamers are chemically synthesized. These sets of variants typically differ from the parent aptamer by the introduction of a single substituent, and differ from each other by the location of this substituent. These variants are then compared to each other and to the parent. Improvements in characteristics may be profound enough that the inclusion of a single substituent may be all that is necessary to achieve a particular therapeutic criterion.

Alternatively the information gleaned from the set of single variants may be used to design further sets of variants in which more than one substituent is introduced simultaneously. In one design strategy, all of the single substituent variants are ranked, the top 4 are chosen and all possible double (6), triple (4) and quadruple (1) combinations of these 4 single substituent variants are synthesized and assayed. In a second design strategy, the best single substituent variant is considered to be the new parent and all possible double substituent variants that include this highest-ranked single substituent variant are synthesized and assayed. Other strategies may be used, and these strategies may be applied repeatedly such that the number of substituents is gradually increased while continuing to identify further-improved variants.

Aptamer Medicinal Chemistry may be used particularly to explore the local, rather than the global, introduction of substituents. Because aptamers are discovered within libraries that are generated by transcription, any substituents that are introduced during the SELEX™ process must be introduced globally. For example, if it is desired to introduce phosphorothioate linkages between nucleotides then they can only be introduced at every A (or every G, C, T, U etc.) (globally substituted). Aptamers which require phosphorothioates at some A's (or some G, C, T, U etc.) (locally substituted) but cannot tolerate it at other As cannot be readily discovered by this process.

The kinds of substituent that can be utilized by the Aptamer Medicinal Chemistry process are only limited by the ability to generate them as solid-phase synthesis reagents and introduce them into an oligomer synthesis scheme. The process is not limited to nucleotides alone. Aptamer Medicinal Chemistry schemes may include substituents that introduce steric bulk, hydrophobicity, hydrophilicity, lipophilicity, lipophobicity, positive charge, negative charge, neutral charge, zwitterions, polarizability, nuclease-resistance, conformational rigidity, conformational flexibility, protein-binding characteristics, mass etc. Aptamer Medicinal Chemistry schemes may include base-modifications, sugar-modifications or phosphodiester linkage-modifications.

When considering the kinds of substituents that are likely to be beneficial within the context of a therapeutic aptamer, it may be desirable to introduce substitutions that fall into one or more of the following categories:

Substituents already present in the body, e.g., 2′-deoxy, 2′-ribo, 2′-O-methyl purines or pyrimidines or 5-methyl cytosine.

Substituents already part of an approved therapeutic, e.g., phosphorothioate-linked oligonucleotides.

Substituents that hydrolyze or degrade to one of the above two categories, e.g., methylphosphonate-linked oligonucleotides.

The anti-IgE aptamers of the invention include aptamers developed through aptamer medicinal chemistry as described herein.

### IgE Specific Binding Aptamers

The materials of the present invention comprise a series of nucleic acid aptamers of 20-50 nucleotides in length which bind specifically to IgE and which, in some embodiments, functionally modulate, e.g., block, the activity of IgE in in vivo and/or functional assays, such as cell based assays.

Aptamers capable of specifically binding and modulating IgE are set forth herein. These aptamers provide a low-toxicity, safe, and effective modality of treating and/or preventing atopic diseases or disorders such as allergic rhinitis (hay fever), atopic dermatitis, asthma, acute urticaria (Wheal-and-Flare), food allergies, and systemic anaphylaxis, which are known to be caused by or otherwise associated with IgE.

Examples of IgE specific binding aptamers for use as therapeutics and/or diagnostics include the following sequences: SEQ ID NOS 11 to 15, 18 to 19, 21, 29, 33, 41 to 44, 46, 50, 56 to 96, 98 to 102, 119 to 124, 126 to 136, 139 to 176, 178 to 190, 194 to 201, 206 to 243, 247, 249 to 259, 261 to 267, 269 to 290, 292 to 296, and 299-336; particularly selected from the group consisting of SEQ ID NOS 29, 33, 41 to 44, 46, 50, 98 to 102, 157 to 176, 178 to 190, 194 to 201, 206 to 219, 293 to 295 and 296; more particularly selected from group consisting of SEQ ID NOS 101, 157, 181, 216, 293 to 295 and 296 are provided.

Other aptamers that bind IgE are described below in Examples 1 to 4.

These aptamers may include modifications as described herein including, e.g., conjugation to lipophilic or high molecular weight compounds such as PEG, incorporation of a CpG motif, incorporation of a capping moiety, incorporation of modified nucleotides, substitutions in the phosphate backbone.

In one embodiment of the invention an isolated, non-naturally occurring aptamer that binds to IgE is provided. In some embodiments, the isolated, non-naturally occurring aptamer has a dissociation constant (“K_{D}”) for IgE of less than 100 μM, less than 1 μM, less than 500 nM, less than 100 nM, less than 50 nM, less than 1 nM, less than 500 μM, less than 100 μM, less than 50 μM, or less than 1 pM. In some embodiments of the invention, the dissociation constant is determined by dot blot assay using a titration of human IgE under the conditions as described in Example 1 below. In a particular embodiment, the dissociation constant is determined by standard dot blot assay, using a titration of human IgE in Dulbecco's PBS (with Mg⁺⁺ and Ca⁺⁺) plus 0.1 mg/mL BSA at room temperature for 30 minutes.

In another embodiment, the aptamer of the invention modulates a function of IgE. In another embodiment, the aptamer of the invention inhibits an IgE function. In yet another embodiment of the invention, the aptamer binds and/or modulates a function of an IgE variant. An IgE variant as used herein encompasses variants that perform essentially the same function as an IgE function, preferably comprises substantially the same structure and in some embodiments comprises at least 70% sequence identity, preferably at least 80% sequence identity, more preferably at least 90% sequence identity, and more preferably at least 95% sequence identity to the amino acid sequence of IgE. In some embodiments of the invention, the sequence identity of target variants is determined using BLAST as described below.

The terms “sequence identity” or “% identity” in the context of two or more nucleic acid or protein sequences, refer to two or more sequences or subsequences that are the same or have a specified percentage of amino acid residues or nucleotides that are the same, when compared and aligned for maximum correspondence, as measured using one of the following sequence comparison algorithms or by visual inspection. For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters. Optimal alignment of sequences for comparison can be conducted, e.g., by the local homology algorithm of Smith & Waterman, Adv. Appl. Math. 2: 482 (1981), by the homology alignment algorithm of Needleman & Wunsch, J Mol. Biol. 48: 443 (1970), by the search for similarity method of Pearson & Lipman, Proc. Nat'l. Acad. Sci. USA 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Ausubel et al., infra).

One example of an algorithm that is suitable for determining percent sequence identity is the algorithm used in the basic local alignment search tool (hereinafter “BLAST”), see, e.g. Altschul et al., J Mol. Biol. 215: 403-410 (1990) and Altschul et al., Nucleic Acids Res., 15: 3389-3402 (1997). Software for performing BLAST analyses is publicly available through the National Center for Biotechnology Information (hereinafter “NCBI”). The default parameters used in determining sequence identity using the software available from NCBI, e.g., BLASTN (for nucleotide sequences) and BLASTP (for amino acid sequences) are described in McGinnis et al., Nucleic Acids Res., 32: W20-W25 (2004).

In another embodiment of the invention, the aptamer has substantially the same ability to bind IgE as that of an aptamer according to any one of SEQ ID NOS 11 to 15, 18 to 19, 21, 29, 33, 41 to 44, 46, 50, 56 to 96, 98 to 102, 119 to 124, 126 to 136, 139 to 176, 178 to 190, 194 to 201, 206 to 243, 247, 249 to 259, 261 to 267, 269 to 290, 292 to 296, and 299-336. In another embodiment of the invention, the aptamer has substantially the same structure and/or ability to bind IgE as that of an aptamer comprising any one of SEQ ID NOS of SEQ ID NOS I Ito 15, 18 to 19, 21, 29, 33, 41 to 44, 46, 50, 56 to 96, 98 to 102, 119 to 124, 126 to 136, 139 to 176, 178 to 190, 194 to 201, 206 to 243, 247, 249 to 259, 261 to 267, 269 to 290, 292 to 296, and 299-336. In another embodiment, the aptamers according to any one of SEQ ID NOS of SEQ ID NOS 11 to 15, 18 to 19, 21, 29, 33, 41 to 44, 46, 50, 56 to 96, 98 to 102, 119 to 124, 126 to 136, 139 to 176, 178 to 190, 194 to 201, 206 to 243, 247, 249 to 259, 261 to 267, 269 to 290, 292 to 296 and 299-336 are provided. In a particular embodiment an aptamer according to any one of SEQ ID NOS 101, 157, 181, 216, 293 to 295 and 296 are provided. In another embodiment, the aptamers of the invention are used as an active ingredient in pharmaceutical compositions. In another embodiment, the aptamers or compositions comprising the aptamers of the invention are used to treat atopic diseases or disorders such as allergic rhinitis (hay fever), atopic dermatitis, asthma, acute urticaria (Wheal-and-Flare), food allergies, peanut allergy, systemic anaphylaxis, allergic conjunctivitis, venial keratoconjunctivitis, atopic keratoconjunctivitis, giant papillary conjunctivitis, and eosinophilic gastroenteritis.

In some embodiments aptamer therapeutics of the present invention have great affinity and specificity to their targets while reducing the deleterious side effects from non-naturally occurring nucleotide substitutions if the aptamer therapeutics break down in the body of patients or subjects. In some embodiments, the therapeutic compositions containing the aptamer therapeutics of the present invention are free of or have a reduced amount of fluorinated nucleotides.

The aptamers of the present invention can be synthesized using any oligonucleotide synthesis techniques known in the art including solid phase oligonucleotide synthesis techniques well known in the art (see, e.g., Froehler et al., Nucl. Acid Res. 14:5399-5467 (1986) and Froehler et al., Tet. Lett. 27:5575-5578 (1986)) and solution phase methods such as triester synthesis methods (see, e.g., Sood et al., Nucl. Acid Res. 4:2557 (1977) and Hirose et al., Tet. Lett., 28:2449 (1978)).

### Aptamers Having Immunostimulatory Motifs

The present invention provides aptamers that bind to IgE and modulate their biological function. More specifically, the present invention provides aptamers that interfere with the binding of IgE to the IgE receptor, FcεRI, thereby preventing IgE mediated allergic reactions. The therapeutic potential of such aptamers can be further enhanced by selecting for aptamers which bind to IgE and contain immunostimulatory or immunomodulatory motifs, or by treating with aptamers which bind to IgE in conjunction with aptamers to a target known to bind immunostimulatory and/or immunomodulatory sequences.

Recognition of bacterial DNA by the vertebrate immune system is based on the recognition of unmethylated CG dinucleotides in particular sequence contexts (“CpG motifs”). One receptor that recognizes such a motif is Toll-like receptor 9 (“TLR 9”), a member of a family of Toll-like receptors (˜10 members) that participate in the innate immune response by recognizing distinct microbial components. TLR 9 binds unmethylated oligodeoxynucleotide (“ODN”) CpG sequences in a sequence-specific manner. The recognition of CpG motifs triggers defense mechanisms leading to innate and ultimately acquired immune responses. For example, activation of TLR 9 in mice induces activation of antigen presenting cells, Lip regulation of MHC class I and II molecules and expression of important co-stimulatory molecules and cytokines including IL-12 and IL-23. This activation both directly and indirectly enhances B and T cell responses, including robust up regulation of the TH1 cytokine IFN-gamma. Collectively, the response to CpG sequences leads to: protection against infectious diseases, improved immune response to vaccines, an effective response against asthma, and improved antibody-dependent cell-mediated cytotoxicity. Thus, CpG ODNs can provide protection against infectious diseases, function as immuno-adjuvants or cancer therapeutics (monotherapy or in combination with a mAb or other therapies), and can decrease asthma and allergic response.

Aptamers of the present invention comprising one or more CpG or other immunostimulatory sequences can be identified or generated by a variety of strategies using, e.g., the SELEX™ process described herein. In general the strategies can be divided into two groups. In group one, the strategies are directed to identifying or generating aptamers comprising both a CpG motif or other immunostimulatory sequence as well as a binding site for a target, where the target (hereinafter “non-CpG target”) is a target other than one known to recognize CpG motifs or other immunostimulatory sequences and known to stimulates an immune response upon binding to a CpG motif. In some embodiments of the invention the non-CpG target is IgE. The first strategy of this group comprises performing SELEX™ to obtain an aptamer to a specific non-CpG target, preferably a target, e.g., IgE, where an immune response is relevant to disease development, using an oligonucleotide pool wherein a CpG motif has been incorporated into each member of the pool as, or as part of, a fixed region, e.g., in some embodiments the randomized region of the pool members comprises a fixed region having a CpG motif incorporated therein, and identifying an aptamer comprising a CpG motif. The second strategy of this group comprises performing SELEX™ to obtain an aptamer to a specific non-CpG target preferably a target, e.g., IgE, where an immune response is relevant to disease development, and following selection appending a CpG motif to the 5′ and/or 3′ end or engineering a CpG motif into a region, preferably a non-essential region, of the aptamer. The third strategy of this group comprises performing SELEX™ to obtain an aptamer to a specific non-CpG target, preferably a target, e.g., IgE, where an immune response is relevant to disease development, wherein during synthesis of the pool the molar ratio of the various nucleotides is biased in one or more nucleotide addition steps so that the randomized region of each member of the pool is enriched in CpG motifs, and identifying an aptamer comprising a CpG motif. The fourth strategy of this group comprises performing SELEX™ to obtain an aptamer to a specific non-CpG target, preferably a target, e.g., IgE, where an immune response is relevant to disease development, and identifying an aptamer comprising a CpG motif. The fifth strategy of this group comprises performing SELEX™ to obtain an aptamer to a specific non-CpG target, preferably a target, e.g., IgE, where a repressed immune response is relevant to disease development, and identifying an aptamer which, upon binding, stimulates an immune response but which does not comprise a CpG motif.

In group two, the strategies are directed to identifying or generating aptamers comprising a CpG motif and/or other sequences that are bound by the receptors for the CpG motifs (e.g., TLR9 or the other toll-like receptors) and upon binding stimulate an immune response. The first strategy of this group comprises performing SELEX™ to obtain an aptamer to a target known to bind to CpG motifs or other immunostimulatory sequences and upon binding stimulate an immune response using an oligonucleotide pool wherein a CpG motif has been incorporated into each member of the pool as, or as part of, a fixed region, e.g., in some embodiments the randomized region of the pool members comprise a fixed region having a CpG motif incorporated therein, and identifying an aptamer comprising a CpG motif. The second strategy of this group comprises performing SELEX™ to obtain an aptamer to a target known to bind to CpG motifs or other immunostimulatory sequences and upon binding stimulate an immune response and then appending a CpG motif to the 5′ and/or 3′ end or engineering a CpG motif into a region, preferably a non-essential region, of the aptamer. The third strategy of this group comprises performing SELEX™ to obtain an aptamer to a target known to bind to CpG motifs or other immunostimulatory sequences and upon binding stimulate an immune response wherein during synthesis of the pool, the molar ratio of the various nucleotides is biased in one or more nucleotide addition steps so that the randomized region of each member of the pool is enriched in CpG motifs, and identifying an aptamer comprising a CpG motif. The fourth strategy of this group comprises performing SELEX™ to obtain an aptamer to a target known to bind to CpG motifs or other immunostimulatory sequences and upon binding stimulate an immune response and identifying an aptamer comprising a CpG motif. The fifth strategy of this group comprises performing SELEX™ to obtain an aptamer to a target known to bind to CpG motifs or other immunostimulatory sequences, and identifying an aptamer which upon binding, stimulate an immune response but which does not comprise a CpG motif.

A variety of different classes of CpG motifs have been identified, each resulting upon recognition in a different cascade of events, release of cytokines and other molecules, and activation of certain cell types. See, e.g., CpG Motifs in Bacterial DNA and Their Immune Effects, Annu. Rev. Immunol. 2002, 20:709-760, incorporated herein by reference. Additional immunostimulatory motifs are disclosed in the following U.S. patents, each of which is incorporated herein by reference: U.S. Pat. No. 6,207,646; U.S. Pat. No. 6,239,116; U.S. Pat. No. 6,429,199; U.S. Pat. No. 6,214,806; U.S. Pat. No. 6,653,292; U.S. Pat. No. 6,426,434; U.S. Pat. No. 6,514,948 and U.S. Pat. No. 6,498,148. Any of these CpG or other immunostimulatory motifs can be incorporated into an aptamer. The choice of aptamers is dependent on the disease or disorder to be treated. Preferred immunostimulatory motifs are as follows (shown 5′ to 3′ left to right) wherein “r” designates a purine, “y” designates a pyrimidine, and “X” designates any nucleotide: AACGTTCGAG (SEQ ID NO 4); AACGTT; ACGT, rCGy; rrCGyy, XCGX, XXCGXX, and X₁X₂CGY₁Y₂ wherein X₁ is G or A, X₂ is not C, Y₁ is not G and Y₂ is preferably T.

In those instances where a CpG motif is incorporated into an aptamer that binds to a specific target other than a target known to bind to CpG motifs and upon binding stimulate an immune response (a “non-CpG target”), the CpG is preferably located in a non-essential region of the aptamer. Non-essential regions of aptamers can be identified by site-directed mutagenesis, deletion analyses and/or substitution analyses. However, any location that does not significantly interfere with the ability of the aptamer to bind to the non-CpG target may be used. In addition to being embedded within the aptamer sequence, the CpG motif may be appended to either or both of the 5′ and 3′ ends or otherwise attached to the aptamer. Any location or means of attachment may be used so long as the ability of the aptamer to bind to the non-CpG target is not significantly interfered with.

As used herein, “stimulation of an immune response” can mean either (1) the induction of a specific response (e.g., induction of a Th1 response) or of the production of certain molecules or (2) the inhibition or suppression of a specific response (e.g., inhibition or suppression of the Th2 response) or of certain molecules.

### Pharmaceutical Compositions

The invention also includes pharmaceutical compositions containing aptamer molecules that bind to IgE. In some embodiments, the compositions are suitable for internal use and include an effective amount of a pharmacologically active compound of the invention, alone or in combination, with one or more pharmaceutically acceptable carriers. The compounds are especially useful in that they have very low, if any toxicity.

Compositions of the invention can be used to treat, prevent and/or ameliorate a pathology, such as a disease or disorder, or alleviate the symptoms of such disease or disorder in a patient. For example, compositions of the present invention can be used to treat, prevent and/or ameliorate a pathology associated with atopic diseases or disorders such as allergic rhinitis (hay fever), atopic dermatitis, asthma, acute urticaria (Wheal-and-Flare), food allergies, and systemic anaphylaxis, which are known to be caused by or otherwise associated with IgE.

Compositions of the invention are useful for administration to a subject suffering from, or predisposed to, a disease or disorder which is related to or derived from a target to which the aptamers of the invention specifically bind. Compositions of the invention can be used in a method for treating a patient or subject having a pathology. The method involves administering to the patient or subject an aptamer or a composition comprising aptamers that bind IgE, so that binding of the aptamer to IgE alters the biological function of the target, thereby treating the pathology.

The patient or subject having a pathology, i.e., the patient or subject treated by the methods of this invention, can be a vertebrate, more particularly a mammal, or more particularly a human.

In practice, the aptamers or their pharmaceutically acceptable salts, are administered in amounts which will be sufficient to exert their desired biological activity, e.g., inhibiting the binding of the IgE aptamer to FcεRI.

One aspect of the invention comprises an aptamer composition of the invention in combination with other treatments for IgE mediated disorders. The aptamer composition of the invention may contain, for example, more than one aptamer. In some examples, an aptamer composition of the invention, containing one or more compounds of the invention, is administered in combination with another useful composition such as an anti-inflammatory agent, an immunosuppressant, an antiviral agent, or the like. In general, the currently available dosage forms of the known therapeutic agents for use in such combinations will be suitable.

“Combination therapy” (or “co-therapy”) includes the administration of an aptamer composition of the invention and at least a second agent as part of a specific treatment regimen intended to provide the beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days or weeks depending upon the combination selected).

“Combination therapy” may, but generally is not, intended to encompass the administration of two or more of these therapeutic agents as part of separate monotherapy regimens that incidentally and arbitrarily result in the combinations of the present invention. “Combination therapy” is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents.

Sequential or substantially simultaneous administration of each therapeutic agent can be effected by any appropriate route including, but not limited to, topical routes, oral routes, intravenous routes, intramuscular routes, and direct absorption through mucous membrane tissues. The therapeutic agents can be administered by the same route or by different routes. For example, a first therapeutic agent of the combination selected may be administered by injection while the other therapeutic agents of the combination may be administered topically.

Alternatively, for example, all therapeutic agents may be administered topically or all therapeutic agents may be administered by injection. The sequence in which the therapeutic agents are administered is not narrowly critical unless noted otherwise. “Combination therapy” also can embrace the administration of the therapeutic agents as described above in further combination with other biologically active ingredients. Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

Therapeutic or pharmacological compositions of the present invention will generally comprise an effective amount of the active component(s) of the therapy, dissolved or dispersed in a pharmaceutically acceptable medium. Pharmaceutically acceptable media or carriers include any and all solvents, dispersion media, coatings, antibacterial and antifungal agents, isotonic and absorption delaying agents and the like. The use of such media and agents for pharmaceutical active substances is well known in the art. Supplementary active ingredients can also be incorporated into the therapeutic compositions of the present invention.

The preparation of pharmaceutical or pharmacological compositions will be known to those of skill in the art in light of the present disclosure. Typically, such compositions may be prepared as injectables, either as liquid solutions or suspensions; solid forms suitable for solution in, or suspension in, liquid prior to injection; as tablets or other solids for oral administration; as time release capsules; or in any other form currently used, including eye drops, creams, lotions, salves, inhalants and the like. The use of sterile formulations, such as saline-based washes, by surgeons, physicians or health care workers to treat a particular area in the operating field may also be particularly useful. Compositions may also be delivered via microdevice, microparticle or sponge.

Upon formulation, therapeutics will be administered in a manner compatible with the dosage formulation, and in such amount as is pharmacologically effective. The formulations are easily administered in a variety of dosage forms, such as the type of injectable solutions described above, but drug release capsules and the like can also be employed.

In this context, the quantity of active ingredient and volume of composition to be administered depends on the host animal to be treated. Precise amounts of active compound required for administration depend on the judgment of the practitioner and are peculiar to each individual.

A minimal volume of a composition required to disperse the active compounds is typically utilized. Suitable regimes for administration are also variable, but would be typified by initially administering the compound and monitoring the results and then giving further controlled doses at further intervals.

For instance, for oral administration in the form of a tablet or capsule (e.g., a gelatin capsule), the active drug component can be combined with an oral, non-toxic, pharmaceutically acceptable inert carrier such as ethanol, glycerol, water and the like. Moreover, when desired or necessary, suitable binders, lubricants, disintegrating agents, and coloring agents can also be incorporated into the mixture. Suitable binders include starch, magnesium aluminum silicate, starch paste, gelatin, methylcellulose, sodium carboxymethylcellulose and/or polyvinylpyrrolidone, natural sugars such as glucose or beta-lactose, corn sweeteners, natural and synthetic gums such as acacia, tragacanth or sodium alginate, polyethylene glycol, waxes, and the like. Lubricants used in these dosage forms include sodium oleate, sodium stearate, magnesium stearate, sodium benzoate, sodium acetate, sodium chloride, silica, talcum, stearic acid, its magnesium or calcium salt and/or polyethyleneglycol, and the like. Disintegrators include, without limitation, starch, methyl cellulose, agar, bentonite, xanthan gum starches, agar, alginic acid or its sodium salt, or effervescent mixtures, and the like. Diluents, include, e.g., lactose, dextrose, sucrose, mannitol, sorbitol, cellulose and/or glycine.

The compounds of the invention can also be administered in such oral dosage forms as timed release and sustained release tablets or capsules, pills, powders, granules, elixirs, tinctures, suspensions, syrups and emulsions. Suppositories are advantageously prepared from fatty emulsions or suspensions.

The pharmaceutical compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are prepared according to conventional mixing, granulating, or coating methods, and typically contain about 0.1% to 75%, preferably about 1% to 50%, of the active ingredient.

Liquid, particularly injectable compositions can, for example, be prepared by dissolving, dispersing, etc. The active compound is dissolved in or mixed with a pharmaceutically pure solvent such as, for example, water, saline, aqueous dextrose, glycerol, ethanol, and the like, to thereby form the injectable solution or suspension. Additionally, solid forms suitable for dissolving in liquid prior to injection can be formulated.

The compounds of the present invention can be administered in intravenous (both bolus and infusion), intraperitoneal, subcutaneous or intramuscular form, all using forms well known to those of ordinary skill in the pharmaceutical arts. Injectables can be prepared in conventional forms, either as liquid solutions or suspensions.

Parenteral injectable administration is generally used for subcutaneous, intramuscular or intravenous injections and infusions. Additionally, one approach for parenteral administration employs the implantation of a slow-release or sustained-released systems, which assures that a constant level of dosage is maintained, according to U.S. Pat. No. 3,710,795, incorporated herein by reference.

Furthermore, preferred compounds for the present invention can be administered in intranasal form via topical use of suitable intranasal vehicles, inhalants, or via transdermal routes, using those forms of transdermal skin patches well known to those of ordinary skill in that art. To be administered in the form of a transdermal delivery system, the dosage administration will, of course, be continuous rather than intermittent throughout the dosage regimen. Other preferred topical preparations include creams, ointments, lotions, aerosol sprays and gels, wherein the concentration of active ingredient would typically range from 0.01% to 15%, w/w or w/v.

For solid compositions, excipients include pharmaceutical grades of mannitol, lactose, starch, magnesium stearate, sodium saccharin, talcum, cellulose, glucose, sucrose, magnesium carbonate, and the like. The active compound defined above, may be also formulated as suppositories, using for example, polyalkylene glycols, for example, propylene glycol, as the carrier. In some embodiments, suppositories are advantageously prepared from fatty emulsions or suspensions.

The compounds of the present invention can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described in U.S. Pat. No. 5,262,564. For example, the aptamer molecules described herein can be provided as a complex with a lipophilic compound or non-immunogenic, high molecular weight compound constructed using methods known in the art. An example of nucleic-acid associated complexes is provided in U.S. Pat. No. 6,011,020.

The compounds of the present invention may also be coupled with soluble polymers as targetable drug carriers. Such polymers can include polyvinylpyrrolidone, pyran copolymer, polyhydroxypropyl-methacrylamide-phenol, polyhydroxyethylaspanamidephenol, or polyethyleneoxidepolylysine substituted with palmitoyl residues. Furthermore, the compounds of the present invention may be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels.

If desired, the pharmaceutical composition to be administered may also contain minor amounts of non-toxic auxiliary substances such as wetting or emulsifying agents, pH buffering agents, and other substances such as for example, sodium acetate, and triethanolamine oleate.

The dosage regimen utilizing the aptamers is selected in accordance with a variety of factors including type, species, age, weight, sex and medical condition of the patient; the severity of the condition to be treated; the route of administration; the renal and hepatic function of the patient; and the particular aptamer or salt thereof employed. An ordinarily skilled physician or veterinarian can readily determine and prescribe the effective amount of the drug required to treat, prevent, counter or arrest the progress of the condition.

Oral dosages of the present invention, when used for the indicated effects, will range between about 0.05 to 7500 mg/day orally. The compositions are preferably provided in the form of scored tablets containing 0.5, 1.0, 2.5, 5.0, 10.0, 15.0, 25.0, 50.0, 100.0, 250.0, 500.0 and 1000.0 mg of active ingredient. Infused dosages, intranasal dosages and transdermal dosages will range between 0.05 to 7500 mg/day. Subcutaneous, intravenous and intraperitoneal dosages will range between 0.05 to 3800 mg/day.

Effective plasma levels of the compounds of the present invention range from 0.002 mg/mL to 50 mg/mL.

Compounds of the present invention may be administered in a single daily dose, or the total daily dosage may be administered in divided doses of two, three or four times daily.

### Modulation of Pharmacokinetics and Biodistribution of Aptamer Therapeutics

It is important that the pharmacokinetic properties for all oligonucleotide-based therapeutics, including aptamers, be tailored to match the desired pharmaceutical application. While aptamers directed against extracellular targets do not suffer from difficulties associated with intracellular delivery (as is the case with antisense and RNAi-based therapeutics), such aptamers must still be able to be distributed to target organs and tissues, and remain in the body (unmodified) for a period of time consistent with the desired dosing regimen.

Thus, the present invention provides materials and methods to affect the pharmacokinetics of aptamer compositions, and, in particular, the ability to tune aptamer pharmacokinetics. The tunability of (i.e., the ability to modulate) aptamer pharmacokinetics is achieved through conjugation of modifying moieties (e.g., PEG polymers) to the aptamer and/or the incorporation of modified nucleotides (e.g., 2′-fluoro or 2′-O-methyl) to alter the chemical composition of the nucleic acid. The ability to tune aptamer pharmacokinetics is used in the improvement of existing therapeutic applications, or alternatively, in the development of new therapeutic applications. For example, in some therapeutic applications, e.g., in anti-neoplastic or acute care settings where rapid drug clearance or turn-off may be desired, it is desirable to decrease the residence times of aptamers in the circulation. Alternatively, in other therapeutic applications, e.g., maintenance therapies where systemic circulation of a therapeutic is desired, it may be desirable to increase the residence times of aptamers in circulation.

In addition, the tunability of aptamer pharmacokinetics is used to modify the biodistribution of an aptamer therapeutic in a subject. For example, in some therapeutic applications, it may be desirable to alter the biodistribution of an aptamer therapeutic in an effort to target a particular type of tissue or a specific organ (or set of organs). In these applications, the aptamer therapeutic preferentially accumulates in a specific tissue or organ(s). In other therapeutic applications, it may be desirable to target tissues displaying a cellular marker or a symptom associated with a given disease, cellular injury or other abnormal pathology, such that the aptamer therapeutic preferentially accumulates in the affected tissue. For example, as described in the provisional application U.S. Ser. No. 60/550,790, filed on Mar. 5, 2004, and entitled “Controlled Modulation of the Pharmacokinetics and Biodistribution of Aptamer Therapeutics”, and in the non-provisional application U.S. Ser. No. 10/______, filed on Mar. 7, 2005, and entitled “Controlled Modulation of the Pharmacokinetics and Biodistribution of Aptamer Therapeutics”, PEGylation of an aptamer therapeutic (e.g., PEGylation with a 20 kDa PEG polymer) is used to target inflamed tissues, such that the PEGylated aptamer therapeutic preferentially accumulates in inflamed tissue.

To determine the pharmacokinetic and biodistribution profiles of aptamer therapeutics (e.g., aptamer conjugates or aptamers having altered chemistries, such as modified nucleotides) a variety of parameters are monitored. Such parameters include, for example, the half-life (t_{1/2}), the plasma clearance (C1), the volume of distribution (Vss), the area under the concentration-time curve (AUC), maximum observed serum or plasma concentration (Cₘₐₓ), and the mean residence time (MRT) of an aptamer composition. As used herein, the term “AUC” refers to the area under the plot of the plasma concentration of an aptamer therapeutic versus the time after aptamer administration. The AUC value is used to estimate the bioavailability (i.e., the percentage of administered aptamer therapeutic in the circulation after aptamer administration) and/or total clearance (C1) (i.e., the rate at which the aptamer therapeutic is removed from circulation) of a given aptamer therapeutic. The volume of distribution relates the plasma concentration of an aptamer therapeutic to the amount of aptamer present in the body. The larger the Vss, the more an aptamer is found outside of the plasma (i.e., the more extravasation).

The present invention provides materials and methods to modulate, in a controlled manner, the pharmacokinetics and biodistribution of stabilized aptamer compositions in vivo by conjugating an aptamer to a modulating moiety such as a small molecule, peptide, or polymer terminal group, or by incorporating modified nucleotides into an aptamer. As described herein, conjugation of a modifying moiety and/or altering nucleotide(s) chemical composition alters fundamental aspects of aptamer residence time in circulation and distribution to tissues.

In addition to clearance by nucleases, oligonucleotide therapeutics are subject to elimination via renal filtration. As such, a nuclease-resistant oligonucleotide administered intravenously typically exhibits an in vivo half-life of <10 min, unless filtration can be blocked. This can be accomplished by either facilitating rapid distribution out of the blood stream into tissues or by increasing the apparent molecular weight of the oligonucleotide above the effective size cut-off for the glomerulus. Conjugation of small therapeutics to a PEG polymer (PEGylation), described below, can dramatically lengthen residence times of aptamers in circulation, thereby decreasing dosing frequency and enhancing effectiveness against vascular targets.

Aptamers can be conjugated to a variety of modifying moieties, such as high molecular weight polymers, e.g., PEG; peptides, e.g., Tat (a 13-amino acid fragment of the HIV Tat protein (Vives, et al. (1997), J. Biol. Chem. 272(25): 16010-7)), Ant (a 16-amino acid sequence derived from the third helix of the Drosophila antennapedia homeotic protein (Pietersz, et al. (2001), Vaccine 19(11-12): 1397-405)) and Arg₇ (a short, positively charged cell-permeating peptides composed of polyarginine (Arg₇) (Rothbard, et al. (2000), Nat. Med. 6(11): 1253-7; Rothbard, J et al. (2002), J. Med. Chem. 45(17): 3612-8)); and small molecules, e.g., lipophilic compounds such as cholesterol. Among the various conjugates described herein, in vivo properties of aptamers are altered most profoundly by complexation with PEG groups. For example, complexation of a mixed 2° F. and 2′-OMe modified aptamer therapeutic with a 20 kDa PEG polymer hinders renal filtration and promotes aptamer distribution to both healthy and inflamed tissues. Furthermore, the 20 kDa PEG polymer-aptamer conjugate proves nearly as effective as a 40 kDa PEG polymer in preventing renal filtration of aptamers. While one effect of PEGylation is on aptamer clearance, the prolonged systemic exposure afforded by presence of the 20 kDa moiety also facilitates distribution of aptamer to tissues, particularly those of highly perfused organs and those at the site of inflammation. The aptamer-20 kDa PEG polymer conjugate directs aptamer distribution to the site of inflammation, such that the PEGylated aptamer preferentially accumulates in inflamed tissue. In some instances, the 20 kDa PEGylated aptamer conjugate is able to access the interior of cells, such as, for example, kidney cells.

Modified nucleotides can also be used to modulate the plasma clearance of aptamers. For example, an unconjugated aptamer which incorporates both 2′-F and 2′-OMe stabilizing chemistries, which is typical of current generation aptamers as it exhibits a high degree of nuclease stability in vitro and in vivo, displays rapid loss from plasma (i.e., rapid plasma clearance) and a rapid distribution into tissues, primarily into the kidney, when compared to unmodified aptamer.

### PEG-Derivatized Nucleic Acids

As described above, derivatization of nucleic acids with high molecular weight non-immunogenic polymers has the potential to alter the pharmacokinetic and pharmacodynamic properties of nucleic acids making them more effective therapeutic agents. Favorable changes in activity can include increased resistance to degradation by nucleases, decreased filtration through the kidneys, decreased exposure to the immune system, and altered distribution of the therapeutic through the body.

The aptamer compositions of the invention may be derivatized with polyalkylene glycol (“PAG”) moieties. Examples of PAG-derivatized nucleic acids are found in U.S. patent application Ser. No. 10/718,833, filed on Nov. 21, 2003, which is herein incorporated by reference in its entirety. Typical polymers used in the invention include polyethylene glycol (“PEG”), also known as polyethylene oxide (“PEO”) and polypropylene glycol (including poly isopropylene glycol). Additionally, random or block copolymers of different alkylene oxides (e.g., ethylene oxide and propylene oxide) can be used in many applications. In its most common form, a polyalkylene glycol, such as PEG, is a linear polymer terminated at each end with hydroxyl groups: HO—CH₂CH₂O—(CH₂CH₂O)ₙ—CH₂CH₂—OH. This polymer, alpha-, omega-dihydroxylpolyethylene glycol, can also be represented as HO-PEG-OH, where it is understood that the -PEG- symbol represents the following structural unit: —CH₂CH₂O—(CH₂CH₂O)ₙ—CH₂CH₂— where n typically ranges from about 4 to about 10,000.

As shown, the PEG molecule is di-functional and is sometimes referred to as “PEG diol.” The terminal portions of the PEG molecule are relatively non-reactive hydroxyl moieties, the —OH groups, that can be activated, or converted to functional moieties, for attachment of the PEG to other compounds at reactive sites on the compound. Such activated PEG diols are referred to herein as bi-activated PEGs. For example, the terminal moieties of PEG diol have been functionalized as active carbonate ester for selective reaction with amino moieties by substitution of the relatively non-reactive hydroxyl moieties, —OH, with succinimidyl active ester moieties from N-hydroxy succinimide.

In many applications, it is desirable to cap the PEG molecule on one end with an essentially non-reactive moiety so that the PEG molecule is mono-functional (or mono-activated). In the case of protein therapeutics which generally display multiple reaction sites for activated PEGs, bi-functional activated PEGs lead to extensive cross-linking, yielding poorly functional aggregates. To generate mono-activated PEGs, one hydroxyl moiety on the terminus of the PEG diol molecule typically is substituted with non-reactive methoxy end moiety, —OCH₃. The other, un-capped terminus of the PEG molecule typically is converted to a reactive end moiety that can be activated for attachment at a reactive site on a surface or a molecule such as a protein.

PAGs are polymers which typically have the properties of solubility in water and in many organic solvents, lack of toxicity, and lack of immunogenicity. One use of PAGs is to covalently attach the polymer to insoluble molecules to make the resulting PAG-molecule “conjugate” soluble. For example, it has been shown that the water-insoluble drug paclitaxel, when coupled to PEG, becomes water-soluble. Greenwald, et al., J. Org. Chem., 60:331-336 (1995). PAG conjugates are often used not only to enhance solubility and stability but also to prolong the blood circulation half-life of molecules.

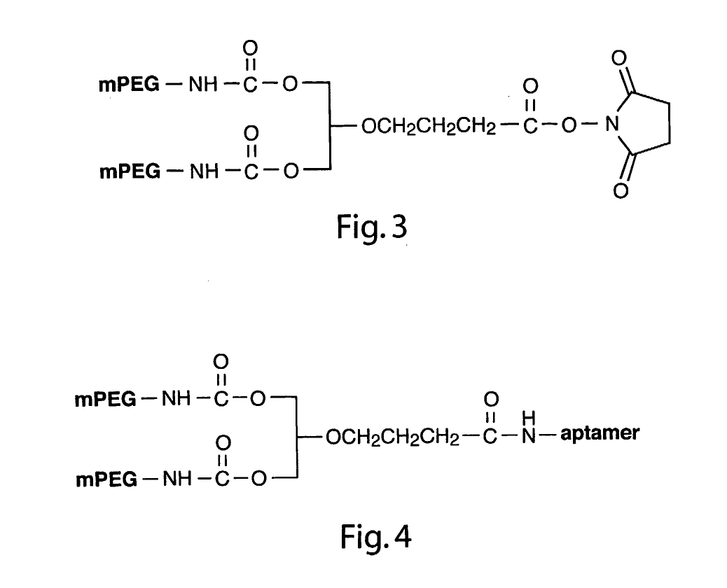

Polyalkylated compounds of the invention are typically between 5 and 80 kDa in size however any size can be used, the choice dependent on the aptamer and application. Other PAG compounds of the invention are between 10 and 80 kDa in size. Still other PAG compounds of the invention are between 10 and 60 kDa in size. For example, a PAG polymer may be at least 10, 20, 30, 40, 50, 60, or 80 kDa in size. Such polymers can be linear or branched. In some embodiments the polymers are PEG. In some embodiment the polymers are branched PEG. In still other embodiments the polymers are 40 kDa branched PEG as depicted in FIG. 3. In some embodiments the 40 kDa branched PEG is attached to the 5′ end of the aptamer as depicted in FIG. 4.

In contrast to biologically-expressed protein therapeutics, nucleic acid therapeutics are typically chemically synthesized from activated monomer nucleotides. PEG-nucleic acid conjugates may be prepared by incorporating the PEG using the same iterative monomer synthesis. For example, PEGs activated by conversion to a phosphoramidite form can be incorporated into solid-phase oligonucleotide synthesis. Alternatively, oligonucleotide synthesis can be completed with site-specific incorporation of a reactive PEG attachment site. Most commonly this has been accomplished by addition of a free primary amine at the 5′-terminus (incorporated using a modifier phosphoramidite in the last coupling step of solid phase synthesis). Using this approach, a reactive PEG (e.g., one which is activated so that it will react and form a bond with an amine) is combined with the purified oligonucleotide and the coupling reaction is carried out in solution.

The ability of PEG conjugation to alter the biodistribution of a therapeutic is related to a number of factors including the apparent size (e.g., as measured in terms of hydrodynamic radius) of the conjugate. Larger conjugates (>10 kDa) are known to more effectively block filtration via the kidney and to consequently increase the serum half-life of small macromolecules (e.g., peptides, antisense oligonucleotides). The ability of PEG conjugates to block filtration has been shown to increase with PEG size up to approximately 50 kDa (further increases have minimal beneficial effect as half life becomes defined by macrophage-mediated metabolism rather than elimination via the kidneys).

Production of high molecular weight PEGs (>10 kDa) can be difficult, inefficient, and expensive. As a route towards the synthesis of high molecular weight PEG-nucleic acid conjugates, previous work has been focused towards the generation of higher molecular weight activated PEGs. One method for generating such molecules involves the formation of a branched activated PEG in which two or more PEGs are attached to a central core carrying the activated group. The terminal portions of these higher molecular weight PEG molecules, i.e., the relatively non-reactive hydroxyl (—OH) moieties, can be activated, or converted to functional moieties, for attachment of one or more of the PEGs to other compounds at reactive sites on the compound. Branched activated PEGs will have more than two termini, and in cases where two or more termini have been activated, such activated higher molecular weight PEG molecules are referred to herein as, multi-activated PEGs. In some cases, not all termini in a branch PEG molecule are activated. In cases where any two termini of a branch PEG molecule are activated, such PEG molecules are referred to as bi-activated PEGs. In some cases where only one terminus in a branch PEG molecule is activated, such PEG molecules are referred to as mono-activated. As an example of this approach, activated PEG prepared by the attachment of two monomethoxy PEGs to a lysine core which is subsequently activated for reaction has been described (Harris et al., Nature, vol. 2: 214-221, 2003).

The present invention provides another cost effective route to the synthesis of high molecular weight PEG-nucleic acid (preferably, aptamer) conjugates including multiply PEGylated nucleic acids. The present invention also encompasses PEG-linked multimeric oligonucleotides, e.g., dimerized aptamers. The present invention also relates to high molecular weight compositions where a PEG stabilizing moiety is a linker which separates different portions of an aptamer, e.g., the PEG is conjugated within a single aptamer sequence, such that the linear arrangement of the high molecular weight aptamer composition is, e.g., nucleic acid-PEG-nucleic acid (-PEG-nucleic acid)ₙ where n is greater than or equal to 1.

High molecular weight compositions of the invention include those having a molecular weight of at least 10 kDa. Compositions typically have a molecular weight between 10 and 80 kDa in size. High molecular weight compositions of the invention are at least 10, 20, 30, 40, 50, 60, or 80 kDa in size.

A stabilizing moiety is a molecule, or portion of a molecule, which improves pharmacokinetic and pharmacodynamic properties of the high molecular weight aptamer compositions of the invention. In some cases, a stabilizing moiety is a molecule or portion of a molecule which brings two or more aptamers, or aptamer domains, into proximity, or provides decreased overall rotational freedom of the high molecular weight aptamer compositions of the invention. A stabilizing moiety can be a polyalkylene glycol, such a polyethylene glycol, which can be linear or branched, a homopolymer or a heteropolymer. Other stabilizing moieties include polymers such as peptide nucleic acids (PNA). Oligonucleotides can also be stabilizing moieties; such oligonucleotides can include modified nucleotides, and/or modified linkages, such as phosphorothioates. A stabilizing moiety can be an integral part of an aptamer composition, i.e., it is covalently bonded to the aptamer.

Compositions of the invention include high molecular weight aptamer compositions in which two or more nucleic acid moieties are covalently conjugated to at least one polyalkylene glycol moiety. The polyalkylene glycol moieties serve as stabilizing moieties. In compositions where a polyalkylene glycol moiety is covalently bound at either end to an aptamer, such that the polyalkylene glycol joins the nucleic acid moieties together in one molecule, the polyalkylene glycol is said to be a linking moiety. In such compositions, the primary structure of the covalent molecule includes the linear arrangement nucleic acid-PAG-nucleic acid. One example is a composition having the primary structure nucleic acid-PEG-nucleic acid. Another example is a linear arrangement of: nucleic acid-PEG-nucleic acid-PEG-nucleic acid.

To produce the nucleic acid-PEG-nucleic acid conjugate, the nucleic acid is originally synthesized such that it bears a single reactive site (e.g., it is mono-activated). In a preferred embodiment, this reactive site is an amino group introduced at the 5′-terminus by addition of a modifier phosphoramidite as the last step in solid phase synthesis of the oligonucleotide. Following deprotection and purification of the modified oligonucleotide, it is reconstituted at high concentration in a solution that minimizes spontaneous hydrolysis of the activated PEG. In a preferred embodiment, the concentration of oligonucleotide is 1 mm and the reconstituted solution contains 200 mM NaHCO₃-buffer, pH 8.3. Synthesis of the conjugate is initiated by slow, step-wise addition of highly purified bi-functional PEG. In a preferred embodiment, the PEG diol is activated at both ends (bi-activated) by derivatization with succinimidyl propionate. Following reaction, the PEG-nucleic acid conjugate is purified by gel electrophoresis or liquid chromatography to separate fully-, partially-, and un-conjugated species. Multiple PAG molecules concatenated (e.g., as random or block copolymers) or smaller PAG chains can be linked to achieve various lengths (or molecular weights). Non-PAG linkers can be used between PAG chains of varying lengths.

The 2′-O-methyl, 2-fluoro and other modified nucleotide modifications stabilize the aptamer against nucleases and increase its half life in vivo. The 3′-3′-dT cap also increases exonuclease resistance. See, e.g., U.S. Pat. Nos. 5,674,685; 5,668,264; 6,207,816; and 6,229,002, each of which is incorporated by reference herein in its entirety.

### PAG-Derivatization of a Reactive Nucleic Acid

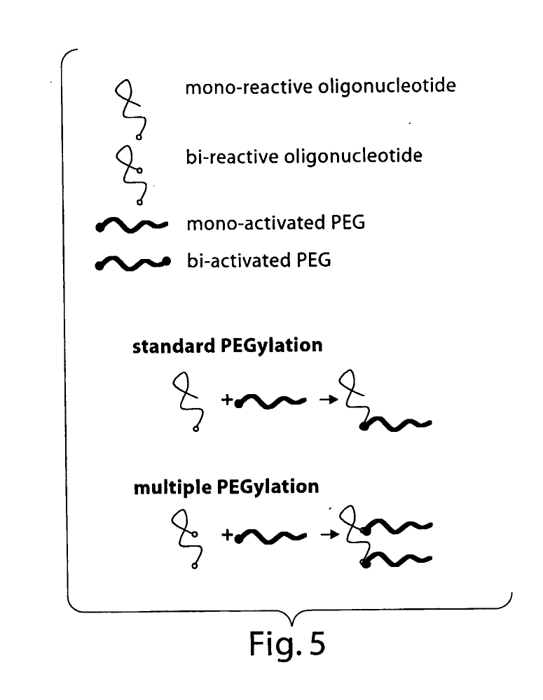

High molecular weight PAG-nucleic acid-PAG conjugates can be prepared by reaction of a mono-functional activated PEG with a nucleic acid containing more than one reactive site. In one embodiment, the nucleic acid is bi-reactive, or bi-activated, and contains two reactive sites: a 5′-amino group and a 3′-amino group introduced into the oligonucleotide through conventional phosphoramidite synthesis, for example: 3′-5′-di-PEGylation as illustrated in FIG. 5. In alternative embodiments, reactive sites can be introduced at internal positions, using for example, the 5-position of pyrimidines, the 8-position of purines, or the 2′-position of ribose as sites for attachment of primary amines. In such embodiments, the nucleic acid can have several activated or reactive sites and is said to be multiply activated. Following synthesis and purification, the modified oligonucleotide is combined with the mono-activated PEG under conditions that promote selective reaction with the oligonucleotide reactive sites while minimizing spontaneous hydrolysis. In the preferred embodiment, monomethoxy-PEG is activated with succinimidyl propionate and the coupled reaction is carried out at pH 8.3. To drive synthesis of the bi-substituted PEG, stoichiometric excess PEG is provided relative to the oligonucleotide. Following reaction, the PEG-nucleic acid conjugate is purified by gel electrophoresis or liquid chromatography to separate fully, partially, and un-conjugated species.

The linking domains can also have one or more polyalkylene glycol moieties attached thereto. Such PAGs can be of varying lengths and may be used in appropriate combinations to achieve the desired molecular weight of the composition.

The effect of a particular linker can be influenced by both its chemical composition and length. A linker that is too long, too short, or forms unfavorable steric and/or ionic interactions with IgE will preclude the formation of complex between the aptamer and IgE. A linker, which is longer than necessary to span the distance between nucleic acids, may reduce binding stability by diminishing the effective concentration of the ligand. Thus, it is often necessary to optimize linker compositions and lengths in order to maximize the affinity of an aptamer to a target.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

### EXAMPLES

### Example 1

### Aptamer Selection and Sequences

### Example 1A

### h-IgE Selection of rRfY IgE Aptamers

Human IgE, purified from human myeloma plasma (hereinafter “h-IgE”), was purchased from Athens Research and Technology (Athens, Ga.). T7 RNA polymerase (Y639F) was expressed and purified. 2′-F pyrimidine nucleotides, and 2′-OMe purine and pyrimidine oligonucleotides were purchased from TriLink BioTechnologies (San Diego, Calif.). All other general reagents were purchased from commercial sources. One selection was performed to identify aptamers to h-IgE using a pool consisting of 2′-OH purine and 2′-F pyrimidine nucleotides (rRfY). A direct selection against h-IgE was performed and yielded high affinity aptamers specific for h-IgE.

Pool Preparation. A DNA template with the sequence 5′-GGGAAAAGCGAATCATACACAAGAN₄₀GCTCCGCCAGAGACCAACCGAGAA-3′ (SEQ ID NO 5) was synthesized using an ABI EXPEDITE™ DNA synthesizer, and deprotected by standard methods. The templates were amplified with the primers 5′ TAATACGACTCACTATAGGGAAAAGCGAATCATACACAAGA 3′ (SEQ ID NO 6) and 5′ TTCTCGGTTGGTCTCTGGCGGAGC 3′ (SEQ ID NO 7) and then used as a template for in vitro transcription with T7 RNA polymerase (Y639F). Transcriptions were done using 40 mM Tris, 40 mM DTT, 1 mM spermidine, 0.002% TritonX-100, 4% PEG-8000, 12 mM MgCl₂, 3 mM 2′-F-CTP, 3 mM 2′-F-UTP, 3 mM GTP, 3 mM ATP, 0.01 units/mL inorganic pyrophosphatase, and T7 polymerase (Y639F), and approximately 0.5 μM template DNA.

Selection. The selection was initiated by incubating of 2×10¹⁴ molecules of 2′-F pyrimidine modified ARC212 pool (5′ GGGAAAAGCGAAUCAUACACAAGA-N₄₀-GCUCCGCCAGAGACCAACCGAGAA 3′) (SEQ ID NO 8) with 100 pmoles of h-IgE protein in a final volume of 100 μL selection buffer (1×SHMCK: 20 mM Hepes, 120 mM NaCl, 5 mM KCl, 1 mM MgCl₂, 1 mM CaCl₂, pH 7.4) for 1 hour at room temperature. RNA-protein complexes and unbound RNA molecules were separated using a 0.45 micron nitrocellulose spin column (Schleicher & Schuell, Keene, N.H.). The column was pre-washed with 1 mL 1×SHMCK buffer, and then the solution containing pool:IgE complexes was added to the column and centrifuged at 1500×g for 2 min. The filter was washed twice with 400 μL 1×SCHMK to remove non-specific binders (Round 1, 2×400 μL 1×SHMCK; in later rounds, 2×500 μL 1×SCHMK). RNA was eluted by addition of 2×200 μL elution buffer (7 M urea, 100 mM sodium acetate, 3 mM EDTA, pre-heated to 95° C.). In later rounds, RNA was eluted with 2×100 μL elution buffer.

Eluted protein was extracted from the RNA mixture with phenol:choloroform, and the pool RNA was precipitated (2 μL glycogen, 1 volume isopropanol). The RNA was reverse transcribed with the ThermoScript RT-PCR™ system (Invitrogen, Carlsbad, Calif.) according to the manufacturer's instructions using the 3′ primer according to SEQ ID NO 7. The cDNA was amplified by PCR (20 mM Tris pH 8.4, 50 mM KCl, 2 mM MgCl₂, 0.5 μM 5′ primer (SEQ ID NO 6), 0.5 μM 3′ primer (SEQ ID NO 7), 0.5 mM each dNTP, 0.05 units/μL Taq polymerase (New England Biolabs, Beverly, Mass.)). The PCR products were purified using the QIAquick PCR purification kit (Qiagen, Valencia, Calif.). Templates were transcribed using α³²P ATP body labeling overnight at 37° C. (4% PEG-8000, 40 mM Tris pH 8.0, 12 mM MgCl₂, 1 mM spermidine, 0.002% Triton x-100, 3 mM 2′OH purines, 3 mM 2′-F CTP and UTP, 25 mM DTT, inorganic pyrophosphatase, T7 RNA polymerase (Y639F) 5 μCi α³²P ATP). The reactions were desalted using Centrisep Spin columns (Princeton Separations, Adelphia, N.J.) according to the manufacturer's instructions and purified on a 1.5 mm denaturing polyacrylamide gel (8 M urea, 10% acrylamide; 19:1 acrylamide:bisacrylamide).

Subsequent rounds were repeated using the same method as for round 1, but with the addition of a negative selection step. Prior to incubation with protein target, the pool RNA was passed through a 0.45 micron nitrocellulose filter column to remove filter binding sequences, then the filtrate was carried on into the positive selection step.

In alternating rounds the pool RNA was gel purified. Transcription reactions were quenched with 50 mM EDTA and ethanol precipitated then purified on a 1.5 mm denaturing polyacrylamide gels. Pool RNA was removed from the gel by electroelution in an Elutrap® apparatus (Schleicher and Schuell, Keene, N.H.) at 225V for 1 hour in 1×TBE (90 mM Tris, 90 mM boric acid, 0.2 mM EDTA). The eluted material was precipitated by the addition of 300 mM sodium acetate and 2.5 volumes of ethanol.

The RNA concentration remained in excess of the h-IgE concentration throughout the selection. The protein concentration was 1 μM for the first 2 rounds, and then was dropped to lower concentrations during subsequent rounds (Table 1). Competitor tRNA was added to the binding reactions at 0.1 mg/mL beginning at Round 4. After 10 rounds of selection were completed, the pool was split into two. Round 11a was conducted with the positive selection having a 10:1 pool to h-IgE concentration ratio. In rounds 11b and 12b at 100:1 RNA to h-IgE concentration ratio was used. This was done to increase stringency in attempts to drive selection towards higher affinity binders. Table 1 contains the selection details including pool RNA concentration, protein concentration, and tRNA concentration used for each round, negative selections step(s) used (if any), and the number of PCR cycles required to obtain a PCR band on a 4% agarose E-Gel (Invitrogen, Carlsbad, Calif.) equal in intensity to the 100 bp marker lane of a 100 bp DNA ladder (˜48 ng of DNA mass) when loaded according to the manufacturer's recommendations (New England Biolabs, Catalog #N3231L, Beverly, Mass.).

The progress of the selection was monitored via measuring the percentage of input pool RNA eluted from the nitrocellulose filter during the positive selection step.

TABLE 1
Conditions used each round of selection using (rRfY)

| | RNA pool | protein | protein | tRNA | Negative | | PCR |
|---|---|---|---|---|---|---|---|
| Round # | conc (μM) | type | conc (μM) | conc (mg/mL) | Selection Step | % elution | cycle # |
| 1 | 3.3 | h-IgE | 1 | 0 | none | 2.44 | 10 |
| 2 | ~1 | h-IgE | 1 | 0 | NC | 0.35 | 15 |
| 3 | 0.8 | h-IgE | 0.75 | 0 | NC | 1.02 | 13 |
| 4 | ~1 | h-IgE | 0.75 | 0.1 | NC | 0.05 | 15 |
| 5 | 1 | h-IgE | 0.75 | 0.1 | NC | 3.80 | 10 |
| 6 | ~1 | h-IgE | 0.5 | 0.1 | NC | 0.04 | 12 |
| 7 | 1 | h-IgE | 0.25 | 0.1 | NC | 3.27 | 8 |
| 8 | ~0.5 | h-IgE | 0.125 | 0.1 | NC | 0.13 | 11 |
| 9 | 0.5 | h-IgE | 0.05 | 0.1 | NC | 3.07 | 8 |
| 10 | ~0.5 | h-IgE | 0.05 | 0.1 | NC | 0.13 | 12 |
| 11a | 0.5 | h-IgE | 0.05 | 0.1 | NC | 7.24 | 8 |
| 11b | 0.5 | h-IgE | 0.005 | 0.1 | NC | 1.03 | 12 |
| 12b | ~0.5 | h-IgE | 0.005 | 0.1 | NC | 0.39 | 12 |

h-IgE Binding Analysis. Dot blot binding assays were performed throughout the selections to monitor the protein binding affinity of the pools. Trace ³²P-labeled pool RNA was combined with h-IgE and incubated at room temperature for 30 min in 1×SHMCK buffer plus 0.1 mg/mL tRNA in a final volume of 25 μL. The mixture was applied to a dot blot apparatus (Schleicher and Schuell Minifold-1 Dot Blot, Acrylic), assembled (from top to bottom) with nitrocellulose, nylon, and gel blot membranes. RNA that is bound to protein is captured on the nitrocellulose filter; whereas the non-protein bound RNA is captured on the nylon filter. When a significant positive ratio of binding of RNA in the presence of h-IgE versus in the absence of h-IgE was seen, the pools were cloned using the TOPO TA cloning kit (Invitrogen, Carlsbad, Calif.) according to the manufacturer's instructions. Round 11a pool templates were cloned and sequenced, and 8 unique clones were assayed in a 1-point dot blot screen (+/−20 nM h-IgE). Round 12b pool was cloned and sequenced, and 4 unique clones were assayed for protein binding in a 1-point dot blot screen (+/−20 nM h-IgE). The percent bound (signal over background) at 20 nM h-IgE for each of clone screened is listed in the far right column in Table 2 below. The sequences for these 12 clones are listed below in Table 3. Based on the 1-point dot blot screen, several clones were selected for K_{D} determination. Clone transcripts were 5′ end labeled with γ-³²P ATP. Binding reactions were prepared under the same conditions used to screen pool affinity as described above: trace ³²P labeled clones were combined with a titration of h-IgE and incubated at room temperature for 30 minutes in 1×SCHMCK buffer plus 0.1 mg/mL tRNA in a final volume of 25 μL. K_{D} values were determined using the dot blot assay for all unique sequences with +/−h-IgE binding ratios>2 in the initial screens by fitting the equation (amp1.1/(1+K_{D1}/[h-IgE])+amp1.2/(1+K_{D2}/[h-IgE]))+background; in which amp1.1 and amp1.2 represent the plateau values for two phases of a biphasic saturation plot and K_{D1} and K_{D2} represent the dissociation constants for each interaction to the resulting data (Kaleidagraph). Results of protein binding characterization are tabulated in Table 2.

TABLE 2
Clone binding activity

| | | | 1-pt Screen Data |
|---|---|---|---|
| SEQ ID | h-IgE K_{D1} | h-IgE K_{D2} | % Bound +/−h-IgE |
| NO | (nM) | (nM) | 20 nM |
| 11 | 0.144 | 12.5 | 4.08 |
| 12 | 0.057 | 9.85 | 4.70 |
| 13 | 0.139 | 14.6 | 5.67 |
| 14 | 1.08 | 99.5 | 2.57 |
| 15 | 0.115 | 19.0 | 3.89 |
| 16 | N.T. | | 1.11 |
| 17 | N.T. | | 0.76 |
| 18 | 1.14 | 27.3 | 4.31 |
| 22 | N.B. | | 0.80 |
| 20 | N.T. | | 1.36 |
| 21 | 0.183 | 27.1 | 2.64 |
| 19 | 0.095 | 17.4 | 3.55 |

N.T. = not tested
N.B. = no significant binding observed

The nucleic acid sequences of the rRfY aptamers characterized in Table 3 are given below. The unique sequence of each aptamer begins at nucleotide 25, immediately following the sequence GGGAAAAGCGAAUCAUACACAAGA (SEQ ID NO 9), and runs until it meets the 3′fixed nucleic acid sequence GCUCCGCCAGAGACCAACCGAGAA (SEQ ID NO 10).

Unless noted otherwise, individual sequences listed below are represented in the 5′ to 3′ orientation and were selected under rRfY SELEX™ conditions wherein the purines (A and G) are 2′-OH and the pyrimidines (U and C) are 2′-fluoro. In some embodiments, the invention comprises aptamers with a nucleic acid sequences as described in Table 3 below. In other embodiments, the nucleic acid sequences of the aptamers described in Table 3 additionally comprise a 3′ cap (e.g., a 3′ inverted dT (3T)), and/or a 5′ amine (NH₂) modification to facilitate chemical coupling, and/or conjugation to a high molecular weight, non-immunogenic compound (e.g., PEG).

TABLE 3
Sequence Information for rRfY aptamers

h-IgE Selection (Round 11a)

SEQ ID NO 11
GGGAAAAGCGAAUCAUACACAAGACGUCGCCAGAUUGAGUGUCGUGGUUC
GGGUUGAGGCGGAAGCUCCGCCAGAGACCAACCGAGAA
SEQ ID NO 12
GGAAAAGCGAAUCAUACACAAGAGUCGCGAUAGAUUGCUUGUGAAUGGUU
UUGGUGGAAGCGGGCUCCGCCAGAGACCAACCGAGAA
SEQ ID NO 13
GGGAAAAGCGAAUCAUACACAAGAGUCGCUAGAUUGCUAGUGUAUGGUUU
AUCUAAAGGCGGCCGCUCCGCCAGAGACCAAGCGAGAA
SEQ ID NO 14
GGGAAAAGCGAAUCAUACACAAGAGGUCUUACAGAUCCUGUGUAGUGGUU
CGAUACAUGCGGGGCUCCGCCAGAGACCAACCGACAA.
SEQ ID NO 15
GGGAAAAGCGAAUCAUACACAAGACGUGAGCAUAUCAUUGAGUGUAGUGG
UUCCGGAGUAAGUCGCUCCGCCAGAGACCAACCGAGAA
SEQ ID NO 16
GGGAAAAGCGAAUCAUACACAAGAGCACCUUGACUGUGAUUCGCGGGUGU
GAGUCGUGCGAAGGCUCCGCCAGAGACCAACCGAGAA
SEQ ID NO 17
GGGAAAAGCGAAUCAUACACAAGAGUGCAAGAAGUGCAUUGCUGUGUCUG
GUUCUUGGCGAUGUGCUCCGCCAGAGACCAACCGAGAA
SEQ ID NO 18
GGGAAAAGCGAAUCAUACACAAGAUCCGAGGGUGGGGAAUAGGCUCACAA
GGGUUUCGCGUGAUGCUCCGCCAGAGACCAACCGAGAA
h-IgE selection (round 12b)
SEQ ID NO 19
GGGAAAAGCGAAUCAUACACAAGAGUGCCGAGGCAUUGCUUGGUAUGGUU
CCGGUCUUGUCGGGGCUCCGCCAGAGACCAACCGAGAA
SEQ ID NO 20
GGGAAAAGCGAAUCAUACACAAGACGUCGCCAGAUUGAGUGUGGUGGUUC
GGGUUGAGGCGGAAGCUCCGCCAGAGACCAACCGAGAA
SEQ ID NO 21
GGGAAAAGCGAAUCAUACACAAGACGUCAGUAAGAUUGAGUGUAUGGUUC
CUGGUGGACAAUAAUGGCUCCGCCAGAGACCAACCGAGAA
SEQ ID NO 22
GGGAAAAGCGAAUCAUACACAAGAGAGUGGAGGAGGUAUGUAUGGUUUGU
GCGUCUGGUGCGGUGCUCCGCCAGAGACCAACCGAGAA

### Example 1B

### Selection of dRmY IgE Aptamers

A selection was performed to identify IgE aptamers containing deoxy-A, G and 2′O-Methyl C, U residues (dRmY composition). This was a direct selection against h-IgE which had been immobilized on a hydrophobic plate. This selection yielded a pool significantly enriched for h-IgE binding versus naïve, unselected pool.

Pool Preparation. A DNA template with the sequence 5′-GGGAGAGGAGAGAACGTTCTACN₃₀CGCTGTCGATCGATCGATCGATG-3′ (SEQ ID NO 23) was synthesized using an ABI EXPEDITE™ DNA synthesizer, and deprotected by standard methods. The templates were amplified with 5′ primer 5′-GGGAGAGGAGAGAACGTTCTAC-3′ (SEQ ID NO 24) and 3′ primer 5′-CATCGATCGATCGATCGACAGC-3′ (SEQ ID NO 25) and then used as a template for in vitro transcription with T7 RNA polymerase (Y639F). Transcriptions were done using 200 mM Hepes, 40 mM DTT, 2 mM spermidine, 0.01% TritonX-100, 10% PEG-8000, 9.6 mM MgCl₂, 2.9 mM MnCl₂, 30 μM GTP, 2 mM mCTP, 2 mM mUTP, 2 mM dGTP, 2 mM dATP, 2 mM GMP, 2 mM spermine, 0.01 units/ul inorganic pyrophosphatase, and T7 polymerase (Y639F).

Selection. Each round of selection was initiated by immobilizing 20 pmoles of h-IgE to the surface of a Nunc Maxisorp (Rochester, NY) hydrophobic plate for 1 hour at room temperature in 100 μL of 1×PBS. The supernatant was then removed and the wells were washed 5 times with 120 μL wash buffer (1×PBS, 0.1 mg/mL tRNA and 0.1 mg/mL ssDNA). In round one, 100 pmoles of pool RNA (6×10¹³ unique molecules) were incubated in 100 μL binding buffer (1×PBS, 0.1 mg/mL tRNA and 0.1 mg/mL ssDNA) in the wells with immobilized protein target for 1 hour at room temperature. The supernatant was then removed and the wells were washed 5 times with 120 μL wash buffer. In subsequent rounds a negative selection step was included; the pool RNA was also incubated for 1 hour at room temperature in empty wells to remove any plastic binding sequences from the pool before the positive selection step. Starting at round 3, a second negative selection step was introduced to further select against non specific binders; the pool was incubated for 1 hour in a well that had been previously blocked with 100 μl blocking buffer (1×PBS, 0.1 mg/mL tRNA, 0.1 mg/mL ssDNA and 0.1 mg/mL BSA). From round 3 forward, the target-immobilized wells were blocked for 1 hour at room temperature in 100 μl blocking buffer (1×PBS, 0.1 mg/mL tRNA, 0.1 mg/mL ssDNA and 0.1 mg/mL BSA) before the positive selection step. In all cases, the pool RNA bound to immobilized h-IgE was reverse transcribed directly in the selection plate after by the addition of RT mix (3′ primer, (SEQ ID NO 25), and Thermoscript RT, Invitrogen) followed by incubation at 65° C. for 1 hour. The resulting cDNA was used as a template for PCR (Taq polymerase, New England Biolabs). “Hot start” PCR conditions coupled with a 68° C. annealing temperature were used to minimize primer-dimer formation. PCR amplification was carried out for the number of cycles (reported in the last column of Table 4 below) required to obtain a PCR band on a 4% agarose E-Gel (Invitrogen, Carlsbad, Calif.) equal in intensity to the 100 bp marker lane of a 100 bp DNA ladder when loaded according to the manufacturer's recommendations (˜48 ng of DNA mass) (New England Biolabs, Catalog #N3231L, Beverly, Mass.). Amplified pool template DNA was desalted with a Micro Bio-Spin column (Bio-Rad, Hercules, Calif.) according to the manufacturer's recommended conditions and used to program transcription of the pool RNA for the next round of selection. The transcribed pool was gel purified using a 10% polyacrylamide gel in each round. Table 4 below shows the conditions used for each round of dRmY aptamer selection.

TABLE 4
Conditions used in each round of selection using dRmY composition

| | RNA pool | protein | protein | tRNA, ssDNA | Negative | PCR |
|---|---|---|---|---|---|---|
| Round # | conc (μM) | type | conc (μM) | conc (mg/mL) | Selection Step | cycle # |
| 1 | 1 | h-IgE | 0.2 | 0.1 | none | 18 |
| 2 | 1 | h-IgE | 0.2 | 0.1 | plate | 18 |
| 3 | 1 | h-IgE | 0.2 | 0.1 | plate, blocking | 20 |
| | | | | | buffer | |
| 4 | 1 | h-IgE | 0.2 | 0.1 | plate, blocking | 17 |
| | | | | | buffer | |
| 5 | 1 | h-IgE | 0.2 | 0.1 | plate, blocking | 17 |
| | | | | | buffer | |
| 6 | 1 | h-IgE | 0.2 | 0.1 | plate, blocking | 15 |
| | | | | | buffer | |
| 7 | 1 | h-IgE | 0.2 | 0.1 | plate, blocking | 16 |
| | | | | | buffer | |

h-IgE Binding Analysis: The selection progress was monitored using a sandwich filter binding assay. The 5′-³²P-labeled pool RNA (trace concentration) was incubated with h-IgE, 1×PBS plus 0.1 mg/mL tRNA, 0.1 mg/mL ssDNA and 0.1 mg/mL BSA for 30 minutes at room temperature and then applied to a nitrocellulose and nylon filter sandwich in a dot blot apparatus (Schleicher and Schuell). The percentage of pool RNA bound to the nitrocellulose was calculated after round 6 and 7 with a seven point screen (0.25 nM, 0.5 nM, 1 nM, 4 nM, 16 nM, 64 nM and 128 nM h-IgE, also a no-target control was run). Pool K_{D} measurements were measured using a titration of protein and the dot blot apparatus as described above.

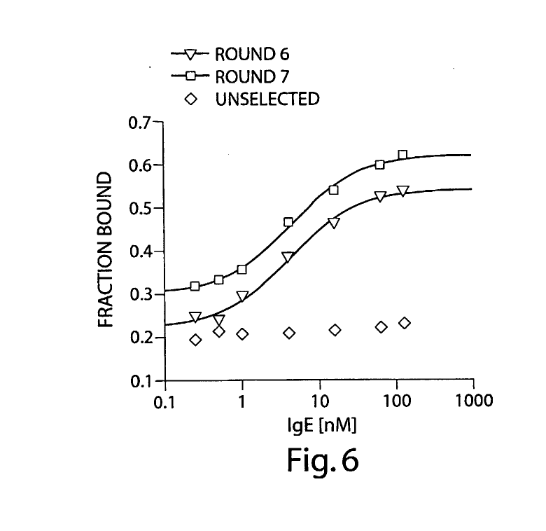

The dRmY h-IgE selection was enriched for h-IgE binding vs. the naïve pool after 6 rounds of selection. At round 6 and round 7 the pool K_{D} was approximately 4 nM. The round 6 pool was cloned using TOPO TA cloning kit (Invitrogen) and 31 individual sequences were generated. There were two dominant clones, represented by 8 and 3 of the 31 sequences, and 21 singletons. FIG. 6 shows a plot of fraction bound versus h-IgE concentration for the round 6 and 7 pools.

Clone screening. For K_{D} determination, clone transcripts of each of the 23 unique sequences were 5′ end labeled with γ-³²P ATP. K_{D} values were determined using an 8 point screen in the dot blot assay (0-300 nM h-IgE, 3 fold serial dilutions), and buffer conditions of 1×Dulbecco's PBS; 1.0 mg/mL tRNA; 0.1 mg/mL sheared salmon sperm DNA; and 0.1 mg/mL BSA. Dissociation constants (K_{D}s) were estimated fitting the data to the equation: fraction RNA bound=amplitude/(1+K_{D}/[h-IgE])+background. Under these binding assay conditions, 20 out of the 23 unique sequences did not show significant binding. Clones according to SEQ ID NO 43 and SEQ ID NO 46 exhibited dissociation constants of 87.7 nM and 109.7 nM respectively.

Each of the 23 unique clones were subsequently re-tested for binding to h-IgE tinder different assay conditions. Clones were made synthetically using standard chemical synthesis and deprotection methods. Clones were then purified by gel electrophoresis. Trace 5′-³²P-labeled aptamers were combined with 7 decreasing concentrations of human IgE starting with 300 nM (3 fold dilutions) and a no protein sample, and incubated at room temperature for 30 minutes in dPBS (contains Mg⁺⁺ and Ca⁺⁺) and 0.1 mg/mL BSA. K_{D} values were determined using the dot blot assay as previously described. The assay was repeated 3 times for each clone. The average percent bound was calculated for each protein concentration and the equilibrium dissociation constants were calculated using the equation:

((A+P+K)−sqrt((A+P+K)̂2−4*A*P))/2A+B; where A=[aptamer]ₜₒₜₐₗ, P=[protein]ₜₒₜₐₗ and B=background signal.

Under these assay conditions, the clones with nucleic acid sequences according to SEQ ID NO 43 and SEQ ID NO 46 showed remarkably improved binding to h-IgE, and six additional clones out of the 23 unique sequences exhibited high affinity binding to h-IgE in the low nanomolar range. Results of protein binding characterization are tabulated in Table 5A, and the sequences for all 22 clones generated are listed below in Table 5B.

TABLE 5A
dRmY Clone Binding Activity in dPBS (with Ca++ and
Mg++), 0.1 mg/mL BSA:

| SEQ ID | | | Error |
|---|---|---|---|
| NO | Aptamer | K_{D} (nM) | (nM) |
| 43 | ARC1991 | 2 | 2 |
| 50 | ARC1992 | 10 | 8 |
| 42 | ARC1993 | 9 | 4 |
| 46 | ARC1994 | 5 | 5 |
| 41 | ARC1995 | 4 | 2 |
| 33 | ARC2001 | 18.0 | 0.1 |
| 44 | ARC2002 | 8 | 6 |
| 29 | ARC2005 | 5 | 3 |

The unique sequence of each aptamer in Table 5B begins at nucleotide 23, immediately following the sequence GGGAGAGGAGAGAACGUUCUAC (SEQ ID NO 26), and runs until it meets the 3′fixed nucleic acid sequence CGCUGUCGAUCGAUCGAUCGAUG (SEQ ID NO 27).

Unless noted otherwise, individual sequences listed below are represented in the 5′ to 3′ orientation and were selected under dRmY SELEX™ conditions wherein the purines (A and G) are deoxy and the pyrimidines (U and C) are 2′-OMe. In some embodiments, the invention comprises aptamers with a nucleic acid sequences as described in Table 5B below. In other embodiments, the nucleic acid sequences of the aptamers described in Table 5B additionally comprise a 3′ cap (e.g., a 3′ inverted dT (3T)), and/or a 5′ amine (NH₂) modification to facilitate chemical coupling, and/or conjugation to a high molecular weight, non-immunogenic compound (e.g., PEG).

TABLE 5B

Unique sequences from Round 6 pool
(all are dRmY composition):

SEQ ID NO 28
GGGAGAGGAGAGAACGUUCUACGAUUAGCAGGGAGGGAGAGUGCGAAGAG
GACGCUGUCGAUCGAUCGAUCGAUG
SEQ ID NO 29
GGGAGAGGAGAGAACGUUCUACACUCUGGGGACCCGUGGGGGAGUGCAGC
AACGCUGUCGAUCGAUCGAUCGAUG
SEQ ID NO 30
GGGAGAGGAGAGAACGUUCUACGAGGUGAGGGUCUACAAUGGAGGGAUGG
UCGCUGUCGAUCGAUCGAUCGAUG
SEQ ID NO 31
GGGAGAGGAGAGAACGUUCUACCCGCAGCAUAGCCUGNGGACCCAUGNGG
GGCGCUGUCGAUCGAUCGAUCGAUG
SEQ ID NO 32
GGGAGAGGAGAGAACGUUCUACUGGGGGGCGUGUUCAUUAGCAGCGUCGU
GUCGCUGUCGA UCGAUCGAUCGAUG
SEQ ID NO 33
GGGAGAGGAGAGAACGUUCUACGCAGCGCAUCUGGGGACCCAAGAGGGGA
UUCGCUGUCGAUCGAUCGAUCGAUG
SEQ ID NO 34
GGGAGAGGAGAGAACGUUCUACGGGAUGGGUAGUUGGAUGGAAAUGGGAA
CGCUGUCGAUCGAUCGAUCGAUG
SEQ ID NO 35
GGGAGAGGAGAGAACGUUCUACGAGGUGUAGGGAUAGAGGGGUGUAGGUA
ACGCUGUCGAUCGAUCGAUCGAUG
SEQ ID NO 36
GGGAGAGGAGAGAACGUUCUACAGGAGUGGAGCUACAGAGAGGGUUAGGG
GUCGCUGUCGAUCGAUCGAUCGAUG
SEQ ID NO 37
GGGAGAGGAGAGAACGUUCUACGGAUGUUGGGAGUGAUAGAAGGAAGGGG
AGCGCUGUCGAUCGAUCGAUCGAUG
SEQ ID NO 38
GGGAGAGGAGAGAACGUUCUACUUGGGGUGGAAGGAGUAAGGGAGGUGCU
GAUCGCUGUCGAUCGAUCGAUCGAUG
SEQ ID NO 39
GGGAGAGGAGAGAACGUUCUACGUAUUAGGGGGGAAGCGGAGGAAUAGAU
CACGCUGUCGAUCGAUCGAUCGAUG
SEQ ID NO 40
GGGAGAGGAGAGAACGUUCUACAGGGAGAGAGUGUUGAGUGAAGAGGAGG
AGUCGCUGUCGAUCGAUCGAUCGAUG
SEQ ID NO 41
GGGAGAGGAGAGAACGUUCUACAUUGUGCUCCUGGGGCCCAGUGGGGAGC
CACGCUGUCGAUCGAUCGAUCGAUG
SEQ ID NO 42
GGGAGAGGAGAGAACGUUCUACGAGCAGCCCUGGGGCCCGGAGGGGGAUG
GUCGCUGUCGAUCGAUCGAUCGAUG
SEQ ID NO 43
GGGAGAGGAGAGAACGUUCUACAGGCAGUUCUGGGGACCCAUGGGGGAAG
UGCGCUGUCGAUCGAUCGAUCGAUG
SEQ ID NO 44
GGGAGAGGAGAGAACGUUCUACCAACGGCAUCCUGGGCCCCACAGGGGAU
GUCGCUGUCGAUCGAUCGAUCGAUG
SEQ ID NO 45
GGGAGAGGAGAGAACGUUCUACGAGUGGAUAGGGAAGAAGGGGAGUAGUC
ACGCUGUCGAUCGAUCGAUCGAUG
SEQ ID NO 46
GGGAGAGGAGAGAACGUUCUACCCGCAGCAUAGCCUGGGGACCCAUGGGG
GGCGCUGUCGAUCGAUCGAUCGAUG
SEQ ID NO 47
GGGAGAGGAGAGAACGUUCUACGGUCGCGUGUGGGGGACGGAUGGGUAUU
GGUCGCUGUCNAUCGAUCGAUCGAUG
SEQ ID NO 48
GGGAGAGGAGAGAACGUUCUACGGGGUUACGUCGCACGAUACAUGCAUUC
AUCGCUGUCGAUCGAUCGAUCGAUG
SEQ ID NO 49
GGGAGAGGAGAGAACGUUCUACUAGCGAGGAGGGGUUUUCUAUUUUUGCG
AUCGCUGUCGAUCGAUCGAUCGAUG
SEQ ID NO 50
GGGAGAGGAGAGAACGUUCUACAAGCAGUUCUGGGGACCCAUGGGGGAAG
UGCGCUGUCGAUCGAUCGAUCGAUG

### Example 1C

### Selection of rRmY h-IgE Aptamers

A selection was performed to identify h-IgE aptamers containing 2′-ribo G and A and 2′-OMethyl C and U residues (rRmY composition). This was a direct selection against h-IgE which had been immobilized on a hydrophobic plate. This selection yielded a pool significantly enriched for h-IgE binding versus naïve, unselected pool.

Pool Preparation. A DNA template with the sequence 5′-GGGAGAGGAGAGAACGTTCTACN₃₀CCCTGTCGATCGATCGATCGATG-3′ (SEQ ID NO 51) was synthesized using an ABI EXPEDITE™ DNA synthesizer, and deprotected by standard methods. The templates were amplified with 5′ primer 5′-GGGAGAGGAGAGAACGTTCTAC-3′ (SEQ ID NO 52) and 3′ primer 5′-CATCGATCGATCGATCGACAGC-3′ (SEQ ID NO 53) and then used as a template for in vitro transcription with T7 RNA polymerase (Y639F). Transcriptions were done using 200 mM Hepes, 40 mM DTT, 2 mM spermidine, 0.01% TritonX-100, 10% PEG-8000, 5 mM MgCl₂, 1.5 mM MnCl₂, 500 μM rGTP, 500 μM rATP, 500 μM mCTP, 500 μM mUTP, 500 μM GMP, 0.01 units/μL inorganic pyrophosphatase, and T7 polymerase (Y639F).

Selection. Each round of selection was initiated by immobilizing 20 pmoles of h-IgE to the surface Nunc Maxisorp hydrophobic plates for 2 hours at room temperature in 100 μL of 1×Dulbecco's PBS. The supernatant was then removed and the wells were washed 4 times with 120 μL wash buffer (1×DPBS, 0.2% BSA, and 0.05% Tween-20). Pool RNA was heated to 90° C. for 3 minutes and cooled to room temperature for 10 minutes to refold. In round 1, a positive selection step was conducted. Briefly, 1×10¹⁴ Molecules (0.2 nmoles) of pool RNA were incubated in 100 μL binding buffer (1×DPBS and 0.05% Tween-20) in the wells with immobilized protein target for 1 hour at room temperature. The supernatant was then removed and the wells were washed 4× with 120 μL wash buffer. In subsequent rounds a negative selection step was included. The pool RNA was also incubated for 30 minutes at room temperature in empty wells to remove any plastic binding sequences from the pool before the positive selection step. The number of washes was increased by 2 additional 120 μl washes (total of 6×120 μl washes) after round 4 to increase stringency. In all cases, the pool RNA bound to immobilized h-IgE was reverse transcribed directly in the selection plate after by the addition of RT mix (3′ primer, (SEQ ID NO 53) and Thermoscript RT, Invitrogen)) followed by incubation at 65° C. for 1 hour. The resulting cDNA was used as a template for PCR (Taq polymerase, New England Biolabs) “Hot start” PCR conditions coupled with a 60° C. annealing temperature were used to minimize primer-dimer formation. Amplified pool template DNA was desalted with a Centrisep column (Princeton Separations) according to the manufacturer's recommended conditions and used to program transcription of the pool RNA for the next round of selection. The transcribed pool was gel purified on a 10% polyacrylamide gel every round. Table 6 below shows the rRmY selection pool h-IgE usage per round.

TABLE 6
rRmY Pool and h-IgE usage per round.

| Round | pmoles of pool used | pmoles of h-IgE used |
|---|---|---|
| 1 | 200 | 20 |
| 2 | 140 | 20 |
| 3 | 115 | 20 |
| 4 | 40 | 20 |
| 5 | 130 | 20 |
| 6 | 80 | 20 |
| 7 | 90 | 20 |

The selection progress was monitored using a sandwich filter binding assay. The 5′-³²P-labeled pool RNA was refolded at 90° C. for 3 minutes and cooled to room temperature for 10 minutes. Next, pool RNA (trace concentration) was incubated with h-IgE in 1×DPBS plus 0.1 mg/mL tRNA for 30 minutes at room temperature and then applied to a nitrocellulose and nylon filter sandwich in a dot blot apparatus (Schleicher and Schuell). The percentage of pool RNA bound to the nitrocellulose was calculated and monitored approximately every 3 rounds with a single point screen (+/−250 nM h-IgE). Pool K_{D} measurements were measured using a titration of protein and the dot blot apparatus as described above.

The selection was enriched after 4 rounds over the naïve pool. The selection stringency was increased by 2 additional 120 μl washes and the selection was continued for 2 more rounds. At round 6 the pool K_{D} was approximately 500 nM. The pools were cloned using TOPO TA cloning kit (Invitrogen) and individual clones sequences were obtained. The round 6 pool contained one dominant clone with a nucleic acid sequence according to SEQ ID NO 56, which made up 71% of the 24 clones sequenced. The dominant clone, as well as three clones that appeared in duplicate, were tested for binding to h-IgE using a 12 point screen (0-250 nM h-IgE in 2 fold serial dilutions). The three duplicate clones showed a higher extent of binding than the dominant clone, however, all of the K_{D}s were approximately 500 nM. An additional set of 96 sequences was obtained, and the dominant clone with a nucleic acid sequence according to SEQ ID NO 56 made up 40% of the 96 clones, along with eight other sequence families which were not evident in the first sequence set. A single point screen was performed on the additional unique sequences (+/−200 nM h-IgE). Based on the single point screen, K_{D}s were determined for an additional 24 K_{D}s sequences using a 12 point screen (0-400 nM h-IgE, 2 fold serial dilutions). The K_{D}s for each of these clones were in excess of 100 nM and further efforts on these clones were terminated. Table 7 below shows the nucleotide sequences of rRmy clones selected.

The unique sequence of each aptamer begins at nucleotide 22, immediately following the sequence GGGAGAGGAGAGAACGUUCUA (SEQ ID NO 54), and runs until it meets the 3′fixed nucleic acid sequence CGCUGUCGAUCGAUCGAUCGAUG (SEQ ID NO 55).

Unless noted otherwise, individual sequences listed below are represented in the 5′ to 3′ orientation and were selected under rRmY SELEX™ conditions wherein the purines (A and G) are 2′-OH and the pyrimidines (U and C) are 2′-OMe. In some embodiments, the invention comprises aptamers with a nucleic acid sequences as described in Table 7 below. In other embodiments, the nucleic acid sequences of the aptamers described in Table 7 additionally comprise a 3′ cap (e.g., a 3′ inverted dT (3T)), and/or a 5′ amine (NH₂) modification to facilitate chemical coupling, and/or conjugation to a high molecular weight, non-immunogenic compound (e.g., PEG).

TABLE 7
rRmY Unique Clone Sequence Information

SEQ ID NO 56
GGGAGAGGAGAGAACGUUCUACGAUCUGGGCGAGCCAGUCUGACUGAGGA
AGCGCUGUCGAUCGAUCGAUCGAUGAAGGGCG
SEQ ID NO 57
GGGAGAGGAGAGAACGUUCUACGCGGUCGGGUGUGUGGAGGAAGUAGUUC
GUCGCUGUCGAUCGAUCGAUCGAUGAAGGGCG
SEQ ID NO 58
GGGAGAGGAGAGAACGUUCUACAGGCGUGUUGGUAGGUACGACGAGGCAU
GCGCUGUCGAUCGAUCGAUCGAGAAGGGCG
SEQ ID NO 59
GGGAGAGGAGAGAACGUUCUACAGGCGUGUUGGUAGGGUACGACGAGGCA
UGCGCUGUCGAUCGAUCGAUCGAUGAAGGGCG
SEQ ID NO 60
GGGAGAGGAGAGAACGUUCUACGAAAAAGAUAUGAGAGAAAGGAUUAAGA
GACGCUGUCGAUCGAUCGAUCGAUGAAGGGCG
SEQ ID NO 61
GGGAGAGGAGAGAACGUUCUACGAAAAAGAUAUGAGAGAAAGGAUUAAGA
GACGCUGUCGAUCGAUCGAUCGAUGAAGGGCG
SEQ ID NO 62
GGGAGAGGAGAGAACGUUCUACGAAGAAGAUAUGAGAGAAAGGAUUAAGA
GACGCUGUCGAUCGAUCGAUCGAUGAAGGGCG
SEQ ID NO 63
GGGAGAGGAGAGAACGUUCUACGAAAAAGAUAUGAGAGAAAGGAUUAAGA
GACGCUGUCCAUCGAUCGAUCGAUGAAGGGCG
SEQ ID NO 64
GGGAGAGGAGAGAACGUUCUACGAAAAAGAUAUGAGAGAAAGGAUUAAGA
GGCGCUGUCGAUCGAUCGAUCGAUGAAGGGCG
SEQ ID NO 65
GGGAGAGGAGAGAACGUUCUACGAAAAAGACAUGAGAGAAAGGAUUAAGA
GACGCUGUCGAUCGAUCGAUCGAUGAAGGGCG
SEQ ID NO 66
GGGAGAGGAGAGAACGUUCUACNAAAAAGUAUAUGAGAGAAAGGAUUAAN
AGACGCUGUCGAUCGAUCGAUCGAUGAAGGGCG
SEQ ID NO 67
GGGAGAGGAGAGAACGUUCUACGAAAAAGAUAUGAGAGAAAAGGAUUGAG
AGAUGCUGUCGAUCGAUCGAUCGAUGAAGGGCG
SEQ ID NO 68
GGGAGAGGAGAGCACGUUCUACGAAAAAGAUAUGGAGAGAAAGGAUUAAG
AGACGCUGUCGAUCGAUCGAUCGAUGAAGGGCG
SEQ ID NO 69
GGGAGAGGAGAGAACGUUCUACGAAAAAGAUAUGAGAGAAAGGAUUAAAA
GAGACUCUGUCGAUCGAUCGAUCGAUGAAGGGCG
SEQ ID NO 70
GGGAGAGGAGAGAACGUUCUACGAANAAGAUACAUAGUAGAAAGGAUUAA
UAAGACGCUGUCGAUCGAUCGAUCGAUGAAGGGCG
SEQ ID NO 71
GGGAGAGGAGAGAACGUUCUACAGGCGUGUUGGUAGGGUACGACGAGGCA
UGCGCUGUCGAUCGAUCGAUCGAUGAAGGGCG
SEQ ID NO 72
GGGAGAGGAGAGAACGUUCUACGCAAAAAUGUGAUGCGAGGUAAUGGACG
CCGCUGUCGAUCGAUCGAUCGAUGAAGGGCG
SEQ ID NO 73
GGGAGAGGAGAGAACGUUCUACGGACCUCAGCGAUAGGGGUUGAAACCGA
CACGCUGUCGAUCGAUCGAUCGAUGAAGGGCG
SEQ ID NO 74
GGGAGAGGAGAGAACGUUCUACAUGGUCGGAUGCUGGGGAGUAGGCAAGG
UUCGCUGUCGAUCGAUCGAUCGAUGAAGGGCG
SEQ ID NO 75
GGGAGAGGAGAGAACGUUCUACGUAUCGGCGAGCGAAGCAUCCGGGAGCG
UUCGCUGUCGAUCGAUCGAUCGAUGAAGGGCG
SEQ ID NO 76
GGGAGAGGAGAGAACGUUCUACGUAUUGGCGCGCGAAGCAUCCGGGAGCG
UUCGCUGUCGAUCGAUCGAUCGAUGAAGGGCG
SEQ ID NO 77
GGGAGAGGAGAGAACGUUCUACUUAUACCUGACGGCCGGAGGCGCAUAGG
UGCGCUGUCGAUCGAUCGAUCGAUGAAGGGCG
SEQ ID NO 78
GGGAGAGGAGAGAACGUUCUACAUGGUCGGAUGCUGGGGAGUAGGCAAGG
UUCGCUGUCGAUCGAUCGAUCGAUGAAGGGCG
SEQ ID NO 79
GGGAGAGGAGAGAACGUUCUACACGAGAGUACUGAGGCGCUUGGUACAGA
GUCGCUGUCGAUCGAUCGAUCGAGAAGGGCG
SEQ ID NO 80
GGGAGAGGAGAGAACGUUCUACAGAAGGUAGAAAAAGGAUAGCUGUGAGA
AGCGCUGUCGAUCGAUCGAUCGAUGAAGGGCG
SEQ ID NO 81
GGGAGAGGAGAGAACGUUCUACUGAGGGAUAAUACGGGUGGAUUGUCUUC
CCGCUGUCGAUCGAUCGAUCGAUGAAGGGCG
SEQ ID NO 82
GGGAGAGGAGAGAACGUUCUACAUUGAGCGUUGAAGUUGGGGAAGCUCCG
AGGCCGCUGUCGAUCGAUCGAUCGAUGAAGGGCG
SEQ ID NO 83
GGGAGAGGAGAGAACGUUCUACGCGGAGAUAUACAGCGAGGUAAUGGAAC
GCCGCUGUCGAUCGAUCGAUCGAUGAAGGGCG
SEQ ID NO 84
GGGAGAGGAGAGAACGUUCUACGAAGACAGCCCAAUAGCGGCACGGAACU
UGCGCUGUCGAUCGAUCGAUCGAUGAAGGGCG
SEQ ID NO 85
GGGAGAGGAGAGAACGUUCUACCGGUUGAGGGCUCGCGUGGAAGGGCCAA
CACGCGCUGUCGAUCGAUCGAUCGAUGAAGGGCG
SEQ ID NO 86
GGGAGAGGAGAGAACGUUCUACAUAUCAAUAGACUCUUGACGUUUGGGUU
UGCGCUGUCGAUCGAUCGAUCGAUGAAGGGCG
SEQ ID NO 87
GGGAGAGGAGAGAACGUUCUACAGUGAAGGAAAAGUAAGUGAAGGUGUGC
GCUGUCGAUCGAUCGAUCGAUGAAGGGCG
SEQ ID NO 88
GGGAGAGGAGAGAACGUUCUACGGAUGAAAUGAGUGUCUGCGAUAGGUUA
AGCGCUGUCGAUCGAUCGAUCGAUGAAGGGCG
SEQ ID NO 89
GGGAGAGGAGAGAACGUUCUACGGAAGGAAAUGUGUGUCUGCGAUAGGUU
AAGCGCUGUCGAUCGAUCGAUCGAUGAAGGGCG

### Example 2

### Aptamer Modification

### Example 2A

### rRfY IgE Clone Minimization

Efforts were taken to minimize the IgE aptamers described above in Example 1A while maintaining, preferably improving, binding affinity. In order to identify the core structural elements required for h-IgE binding, the 3′-boundaries of several of the high affinity h-IgE binders were determined. RNA transcripts were labeled at the 5′-end with γ-³²P ATP and T4 polynucleotide kinase. Radiolabeled ligands were subjected to partial alkaline hydrolysis and then selectively bound in solution to h-IgE at 500 nM before being partitioned over nitrocellulose filters. Retained oligonucleotides were resolved on 8% denaturing polyacrylamide gels. The smallest oligonucleotide bound to h-IgE defined the 3′-boundary. The 3′-boundaries of selected clones are described in Table 8. On the basis of the boundary experiments as well as visual inspection of predicted folds, truncated constructs were prepared and oligos were ordered from Integrated DNA Technologies (Coralville, Iowa). Minimized versions of the parent clones with nucleic acid sequences according to SEQ ID NO 1, SEQ ID NO 18, and SEQ ID NO 21 showed significant protein binding, measured by the sandwich filter binding assay previously described. Minimer binding data are shown in Table 8, while the corresponding sequences are shown in Table 9.

TABLE 8
Minimer binding activity

| | | 3′- | h-IgE | |
|---|---|---|---|---|
| Minimized | | boundary of | Km | h-IgE K_{D2} |
| Clone | Parent Clone | parent | (nM) | (nM) |
| SEQ ID NO 90 | SEQ ID NO 11 | U49 | 0.33 | 28.2 |
| SEQ ID NO 91 | SEQ ID NO 11 | | 0.56 | 36.9 |
| SEQ ID NO 92 | SEQ ID NO 11 | | 0.25 | 22.0 |
| SEQ ID NO 93 | SEQ ID NO 18 | U55 | 0.65 | 17.1 |
| SEQ ID NO 94 | SEQ ID NO 18 | | 1.01 | 26.5 |
| SEQ ID NO 95 | SEQ ID NO 21 | G47 | 4.5 | 117.2 |
| SEQ ID NO 96 | SEQ ID NO 21 | | 0.365 | 39.7 |

*All measurements were done in 1X SHMCK buffer plus 0.1 mg/mL tRNA.

Unless noted otherwise, individual sequences listed below are represented in the 5′ to 3′ orientation and were selected under rRfY SELEX™ wherein all purines (A and G) are 2′-OH, and all pyrimidines (C and U) are 2′-fluoro. In some embodiments, the invention comprises aptamers with a nucleic acid sequences as described in Table 9 below. In other embodiments, the nucleic acid sequences of the aptamers described in Table 9 additionally comprise a 3′ cap (e.g., a 3′ inverted dT (3T)), and/or a 5′ amine (NH₂) modification to facilitate chemical coupling, and/or conjugation to a high molecular weight, non-immunogenic compound (e.g., PEG).

TABLE 9
Sequences of rRfY minimized aptamers

SEQ ID NO 90
GGGAAAAGCGAAUCAUACACAAGACGUCGCCAGAUUGAGUGUCGUGGUU
SEQ ID NO 91
GGAAUCAUACACAAGACGUCGCCAGAUUGAGUGUCGUGGUUCC
SEQ ID NO 92
GGAAUCAUACACAAGACGUCGCCAGAUUGAGUGUCGUGGUU
SEQ ID NO 93
GGAGAUCCGAGGGUGGGCAAUAGGCUCACAAGGGUUU
SEQ ID NO 94
GGAUCCGAGGGUGGGCAAUAGGCUCACAAGGGUCC
SEQ ID NO 95
GGAAUCAUACACAAGACGUCAGUAAGAUUGAGUGUAUGGUUCC
SEQ ID NO 96
GGAAUCAUACACAAGACGUCAGUAAGAUUGAGUGUAUGGUU

### Example 2B

### dRmY IgE Clone Minimization

Efforts were taken to minimize the dRmY IgE aptamers described above in Example 1B while maintaining, preferably improving, binding affinity. On the basis of the inspection of predicted folds for clones with nucleic acid sequences according to SEQ ID NO 43 and SEQ ID NO 46, a panel of minimized sequences was designed. The highest affinity molecule, ARC445 (SEQ ID NO 101) is 23 nucleotides in length and binds h-IgE with a K_{D} of 22 nM. The data are summarized in Table 10. Table 11 shows the nucleotide sequences of ARC441 to ARC447 (SEQ ID NOs 97-103), the truncants derived from the clones with nucleic acid sequences according to SEQ ID NO 43 and SEQ ID NO 46.

TABLE 10
Minimized dRmY h-IgE binders

| Minimer | ARC reference | | |
|---|---|---|---|
| SEQ ID | number for | SEQ ID NO | |
| NO | minimer | Parent clone | K_{D} h-IgE (nM) |
| 97 | ARC441 | SEQ ID NO 43 | N.B. |
| 98 | ARC442 | SEQ ID NO 43 | 174 |
| 99 | ARC443 | SEQ ID NO 43 | 55 |
| 100 | ARC444 | SEQ ID NO 46 | 73 |
| 101 | ARC445 | SEQ ID NO 46 | 22 |
| 102 | ARC446 | SEQ ID NO 46 | 43 |
| 103 | ARC447 | SEQ ID NO 46 | N.B. |

K_{D} measurements were performed in 1X PBS in the presence of 0.1 mg/mL BSA, 1 mg/mL tRNA and 0.1 mg/mL ssDNA at 25° C. for 30 minutes.

Unless noted otherwise, individual sequences listed below are represented in the 5′ to 3′ orientation and were selected under dRmY SELEX™ wherein all purines (A and G) are deoxy, and all pyrimidines (C and U) are 2′-O-methyl. In some embodiments, the invention comprises aptamers with a nucleic acid sequences as described in Table 11 below. In other embodiments, the nucleic acid sequences of the aptamers described in Table 11 additionally comprise a 3′ cap (e.g., a 3′ inverted dT (3T)), and/or a 5′ amine (NH₂) modification to facilitate chemical coupling, and/or conjugation to a high molecular weight, non-immunogenic compound (e.g., PEG).

TABLE 11

Truncants of clones with nucleic acid
sequences according to SEQ ID NO 43
and SEQ ID NO 46

SEQ ID NO 97 (ARC 441)
UUCUGGGGACCCAUGGGGGAA
SEQ ID NO 98 (ARC 442)
GUUCUGGGGACCCAUGGGGGAAC
SEQ ID NO 99 (ARC 443)
AGUUCUGGGGACCCAUGGGGGAACU
SEQ ID NO 100 (ARC 444)
GCCUGGGGACCCAUGGGGGGC
SEQ ID NO 101 (ARC 445)
AGCCUGGGGACCCAUGGGGGGCU
SEQ ID NO 102 (ARC 446)
UAGCCUGGGGACCCAUGGGGGGCUA
SEQ ID NO 103 (ARC 447)
GCCUGGGGAACCAUGGGGGGC

### Example 2C

### Doped Reselection

### ARC445

Doped reselections are used to explore the sequence requirements within an active clone or minimer. During doped reselection, selections are carried out with a synthetic, degenerate pool that has been designed based on a single sequence. The level of degeneracy usually varies from 70% to 85% wild type nucleotide. In general, neutral mutations are observed but in some cases sequence changes can result in improvements in affinity. The composite sequence information can then be used to identify the minimal binding motif and aid in aptamer medicinal chemistry efforts.

A selection using a doped pool based on the minimized h-IgE binding sequence ARC445 (SEQ ID NO 101) (described in Example 2B) was performed in order to identify higher affinity binders. The selection was against h-IgE immobilized to the surface of a hydrophobic plate and utilized techniques designed to drive selection toward higher affinity aptamers such as combinations of multiple longer washes (e.g. 30 minutes, 60 minutes, overnight).

Pool preparation. A DNA template with the sequence 5′-GGGAGAGGAGAGAACGTTCTACAGCCTGGGGACCCATGGGGGGCTGGTCG ATCGATCGATCATCGATG-3′ (SEQ ID NO 104) was synthesized using an ABI EXPEDITE™ DNA synthesizer, and deprotected by standard methods. The nucleotides in bold had an 85% chance of being the indicated residue and a 5% chance of being one of the other 3 nucleotides. The templates were amplified with 5′primer 5′-GGGAGAGGAGAGAACGTTCTAC-3′ (SEQ ID NO 52) and 3′ primer 5′-CATCGATGATCGATCGATCGACC-3′ (SEQ ID NO 105) and then used as a template for in vitro transcription with T7 RNA polymerase (Y639F). Transcriptions were done using 200 mM Hepes, 40 mM DTT, 2 mM spermidine, 0.01% TritonX-100, 10% PEG-8000, 9.6 mM MgCl₂, 2.9 mM MnCl₂, 30 μM GTP, 2 mM mCTP, 2 mM mUTP, 2 mM dGTP, 2 mM dATP, 2 mM GMP, 2 mM spermine, 0.01 units/μl inorganic pyrophosphatase, and T7 polymerase (Y639F).

Selection. Each round of selection was initiated by immobilizing 20 pmoles of h-IgE to the surface of a Nunc Maxisorp hydrophobic plate for 1 hour at room temperature in 100 μL 1×Dulbecco's PBS (DPBS). The supernatant was then removed and the wells were washed 2 times with 120 μL 1×Dulbecco's PBS. The wells were blocked by adding 100 μL blocking buffer (1×Dulbecco's PBS, 0.1 mg/mL tRNA, 0.1 mg/mL salmon sperm DNA, and 0.1 mg/mL BSA) and incubating 1 hour at room temperature. The supernatant was removed and the wells were washed 2 times with 120 μL wash buffer. Starting at round 2, a negative binding incubation for one hour to an empty well, and a negative binding incubation to a well blocked with BSA were both conducted for the RNA pools. The positive selection was conducted by adding 100 pmoles of pool RNA in 100 μL 1×Dulbecco's PBS to the target well. 0.1 mg/mL tRNA and 0.1 mg/mL salmon sperm DNA was also added to the positive selection. After incubation for 1 hour at room temperature the supernatant was removed and the wells were washed five times with 120 μL wash buffer (1×DPBS) as outlined in Table 12 below. Additional selections were added by branching off the selected pool at round 3 and round 4. These were conducted with longer washes to increase selection stringency.

The RNA was reverse transcribed with the ThermoScript RT-PCR™ system (Invitrogen) in a 100 μl reaction volume at 65 degrees for 30 minutes, using the 3′ primer sequence according to SEQ ID NO 5. The cDNA was amplified by PCR (20 mM Tris pH 8.4, 50 mM KCl, 2 mM MgCl₂, 0.5 μM 5′ primer (SEQ ID NO 52), 0.5 μM 3′ primer (SEQ ID NO 105), 0.5 mM each dNTP, 0.05 units/μL Taq polymerase (New England Biolabs) using the number of PCR cycles (last column, Table 12) required to obtain a PCR band on a 4% agarose E-Gel (Invitrogen, Carlsbad, Calif.) equal in intensity to the 100 bp marker lane of a 100 bp DNA ladder when loaded according to the manufacturer's recommendations (˜48 ng of DNA mass) (New England Biolabs, Catalog #N3231L, Beverly, Mass.). The PCR products were then desalted using Centrisep Spin columns (Princeton Separations). Templates were transcribed overnight at 37 degrees using 200 mM Hepes, 40 mM DTT, 2 mM spermidine, 0.01% TritonX-100, 10% PEG-8000, 9.6 mM MgCl₂, 2.9 mM MnCl₂, 30 μM GTP, 2 mM mCTP, 2 mM mUTP, 2 mM dGTP, 2 mM dATP, 2 mM GMP, 2 mM spermine, 0.01 units/μl inorganic pyrophosphatase, and T7 polymerase (Y639F). RNA for subsequent rounds was purified on a 10% polyacryamide gel. Table 12 below shows a summary of the doped reselection profile for ARC445 (SEQ ID NO 101).

TABLE 12
ARC445 doped reselection profile

| | | | | | |
|---|---|---|---|---|---|
| | RNA | Negative | target | | PCR |
| Round | (pmol) | steps | (pmol) | washes | cycles |

Regular conditions

| | | | | | |
|---|---|---|---|---|---|
| 1 | 100 | none | 20 | 120 μl/quick | 19 |
| 2 | 100 | well, BSA | 20 | 120 μl/quick | 10 |
| 3a | 100 | well, BSA | 20 | 120 μl/quick | 10 |
| 4a | 80.64 | well, BSA | 20 | 120 μl/quick | 10 |
| 5a | 100 | well, BSA | 20 | 120 μl/quick | 10 |

LONG WASHES starting at round 3 (split from regular round 3 template)

| | | | | | |
|---|---|---|---|---|---|
| 1 | 100 | none | 20 | 120 μl/quick | 19 |
| 2 | 100 | well, BSA | 20 | 120 μl/quick | 10 |
| 3b | 30.67 | well, BSA | 20 | 120 μl/30 min | 10 |
| 4b | 100 | well, BSA | 20 | 120 μl/ON | 12 |

LONG WASHES starting at round 4 (split from regular round 4 template)

| | | | | | |
|---|---|---|---|---|---|
| 1 | 100 | none | 20 | 120 μl/quick | 19 |
| 2 | 100 | well, BSA | 20 | 120 μl/quick | 10 |
| 3a | 100 | well, BSA | 20 | 120 μl/quick | 10 |
| 4c | 80.64 | well, BSA | 20 | 120 μl/30 min | 10 |
| 5c | 100 | well, BSA | 20 | 120 μl/ON | 12 |

DNA from the final selection rounds from all wash conditions and from round 2 and 3 from the non-stringent selection were cloned using TOPO TA cloning kit (Invitrogen, Carlsbad Calif.). Seven sequences were selected to be synthesized and tested for specific binding to h-IgE using the dot blot assay and conditions previously described in Example 1B above. None of the seven clones tested showed any significant binding to h-IgE. Table 13 below shows the sequences of clones from the ARC445 doped reselection.

Unless noted otherwise, individual sequences listed below are represented in the 5′ to 3′ orientation and were selected under dRmY SELEX™ wherein all purines (A and G) are deoxy, and all pyrimidines (C and U) are 2′-O-methyl. In some embodiments, the invention comprises aptamers with a nucleic acid sequences as described in Table 13 below. In other embodiments, the nucleic acid sequences of the aptamers described in Table 13 additionally comprise a 3′ cap (e.g., a 3′ inverted dT (3T)), and/or a 5′ amine (NH₂) modification to facilitate chemical coupling, and/or conjugation to a high molecular weight, non-immunogenic compound (e.g., PEG).

TABLE 13

Sequences of clones from the ARC445 doped
reselection

SEQ ID NO 106 ARC664
AGCCUGGGGACCCAUGGGGGCU
SEQ ID NO 107 ARC665
CGCCUGGGGACCCAGGGCGGCCU
SEQ ID NO 108 ARC666
ACCCUGGUGGCCCAUGGGGUGCU
SEQ ID NO 109 ARC667
AGCCUGGCCACCCAUGGGGGGUGGU
SEQ ID NO 110 ARC668
AGUCUGGGGACAGAUGGAUGGCU
SEQ ID NO 111 ARC669
AGCUGUGGAGUCGUGUGGGGCU
SEQ ID NO 112 ARC67O
AAGCCUGGGGACCCAUGCGGGGGCU

### Example 2D

### Doped Re-Selection of DNA IgE Aptamers

A selection using a doped pool based on the h-IgE binding sequence D17.4 5′-GGGGCACGTTTATCCGTCCCTCCTAGTGGCGTGCCCC-3′ (SEQ ID NO 113), (U.S. Pat. No. 5,686,592 incorporated herein by reference in its entirety) was performed in order to identify higher affinity binders. The selection was against h-IgE immobilized to the surface of a hydrophobic plate and utilized techniques designed to drive selection toward higher affinity aptamers such as combinations of multiple longer washes (e.g. 30 minutes, 60 minutes, overnight). The experiment yielded a number of D17.4 derivatives with increased affinity for h-IgE.

Pool preparation. The DNA template (ARC 273) with the sequence 5′gatcccttgttcagtccGGGGCACGTTTATCCGTCCCTCCTAGTGGCGT GCCCCttaagccacaggactccaaa-3′ (SEQ ID NO 114) in which the primer binding sites are represented in lower case and the nucleotides in bold had an 85% chance of being the indicated residue and a 5% chance of being one of the other 3 nucleotides. The template was synthesized on an ABI EXPEDITE™ DNA synthesizer, and deprotected by standard methods. The pool was amplified with the 5′ primer 5′-GATCCCTTGTTCAGTCCG-3′ (SEQ ID NO 115) and 3′ primer 5′-GGAGTCCTGTGGCTTArA-3′ (SEQ ID NO 116) (where rA stands for ribo adenosine which enabled primer cleavage post-PCR, allowing template and pool bands to be separated by gel) using standard conditions. The product was subjected to alkaline hydrolysis (200 mM NaOH, 90° C., 15 min) followed by precipitation with isopropanol. The strands were separated on an 8% denaturing polyacrylamide gel and the ssDNA pool, which migrated with a lower mobility, was excised from the gel.

Each round of selection was initiated by immobilizing 20 pmoles of h-IgE to the surface of a Nunc Maxisorp hydrophobic plate for 1 hour at room temperature in 100 μL selection buffer (1×SCHMK; see Example 1A). The supernatant was then removed and the wells were washed 4 times with 120 μL wash buffer (1×SCHMK, 0.2% BSA and 0.5% Tween-20). The wells were blocked by adding 100 μL blocking buffer (1×SHMCK, 1% BSA, 0.5% Tween-20) and incubating 1 hour at room temperature. The supernatant was removed and the wells were washed 4 times with 120 μL wash buffer. In round one, 80 pmoles of pool DNA (1.5×10¹³ unique molecules) in 100 μL selection buffer was incubated in a blank well for 1 hour at room temperature as a negative selection step to eliminate non-specific binders. The supernatant was then removed, 12 μL of 9.1 mg/mL salmon sperm DNA in 1×SCHMK was added, and the mixture was transferred to the well containing target protein. After incubation 1 hour at room temperature the supernatant was removed and the wells were washed multiple times with 120 μL wash buffer. Table 14 below shows the selection conditions for the doped reselection.

TABLE 14
Selection stringency as a function of round and washing

| | | # washes | # washes | |
|---|---|---|---|---|
| | | with 15 | with an | # washes |
| | | minute | overnight | post |
| Round | # initial | incubation | incubation | buffer |
| # | washes | in buffer | in buffer | incubation |
| 1 | 6 | — | — | — |
| 2 | 6 | — | — | — |
| 3 | 6 | — | — | — |
| 4 | 6 | 2 | — | — |
| 5 | 6 | 2 | 1 | 1 |

In all cases, the pool DNA bound to immobilized h-IgE was eluted with 2×150 μL washes of hot elution buffer (7 M Urea, 100 mM NaOAc pH 5, 3 mM EDTA), precipitated by the addition of isopropanol, then amplified by PCR. DNA eluted after round one of selection was amplified and purified as described above for the initial DNA doped pool amplification using the 5′ and 3′ primers according to SEQ ID NO 115 and SEQ ID NO 116 using standard conditions. For rounds 2-4, eluted DNA was amplified using the 5′ and 3′ primers according to SEQ ID NO 115 and 5′-(5-biotin-T)(5-biotin-T)(5-biotin-T)GGAGTCCTGTGGCTTAA-3′ (SEQ ID NO 117). The PCR product was then extracted with phenol and precipitated with ethanol. DNA was then re-suspended in 5-10 μl of 1×SCHMK buffer with addition of 300 pmoles of neutravidin (Pierce, Rockford, Ill.), incubated for 30 min. at room temperature, followed by addition of 10 μl of formamide loading dye and separation on an 8% denaturing polyacrylamide gel. The biotin-neutravidin complex remains intact through denaturation, significantly reducing the mobility of the anti-sense DNA strand relative to the sense DNA strand. Thus the strands were separated and the desired ssDNA pool members, which migrate with a higher mobility, were excised from the gel and carried into the next round of selection.

After selection rounds 3 and 5, the pool templates re-amplified using the 5′ and 3′ primers according to SEQ ID NO 115 and 5′-GGAGTCCTGTGGCTTAA-3′ (SEQ ID NO 118) (which is completely unmodified DNA) and were cloned using TOPO TA cloning kit (Invitrogen). 84 individual sequences were generated. Individual clones were prepared without the primer binding sequences and screened for h-IgE binding at 5 and 50 nM using the dot blot assay set up and conditions previously described in Example 1A. Three clones with nucleic acid sequences according to the following SEQ ID NOs in this initial screen did not show any binding to h-IgE: SEQ ID NO 125, SEQ ID NO 137, and SEQ ID NO 138. K_{D}s were determined for the best binders identified in the initial screen, using a titration of h-IgE (30 pM to 30 nM, 3 fold dilutions) (Table 15). Several clones showed improved binding versus the parent sequence, D17.4 (SEQ ID NO 113). The clone with a nucleic acid sequence according to SEQ ID NO 140 showed among the most significant improvements in affinity relative to the parent sequence D17.4 (SEQ ID NO 113). Interestingly, the differences between the clone with a nucleic acid sequence according to SEQ ID NO 140 and clone D17.4 (SEQ ID NO 113) were quite subtle involving changes only in the proposed Watson/Crick stem directly adjacent to the loop region of the D17.4 aptamer (SEQ ID NO 113). Nearly all of the unique clones from the re-selection (including the clone with a nucleic acid sequence according to SEQ ID NO 140) had acquired mutations in the G8-C30 base pair of D17.4 to become either A8-T30 or C8-G30 effectively switching the functional groups presented in the major groove of the helix at that pairing position from a 5′-strand-carbonyl/3′-strand-amino in the case of G8-C30 to a 5′-strand-amino/3′-strand carbonyl in the cases of both A8-T30 and C8-G30. The highest affinity clones (other than that of the clone according to SEQ ID NO 140) were then redesigned with their own loop sequences (residues 9-29) and an optimized stem sequence based on the stem of the clone with a nucleic acid sequence according to SEQ ID NO 140 (residues 1-8 and 30-37). The C4-C34 mispairing was reverted to G4-C34 in the optimized SEQ ID NO 140 stem. Table 15 below shows nucleotide sequence lengths and affinities of selected DNA aptamers. Table 16 shows the nucleotide sequences of DNA aptamers selected.

TABLE 15
Lengths and binding affinities of selected DNA aptamers

| SEQ ID | | |
|---|---|---|
| NO/ | Length | K_{D} (nM) |
| 113 | 37 | 0.5-2 |
| 132 | 37 | 0.4 |
| 119 | 37 | 0.6 |
| 139 | 36 | 0.4 |
| 133 | 37 | 0.7 |
| 147 | 37 | 0.5 |
| 128 | 37 | 0.5 |
| 140 | 36 | 0.2 |
| 149 | 38 | 0.8 |
| 131 | 37 | 0.3 |
| 150 | 37 | 0.7 |
| 151 | 37 | 0.5 |
| 152 | 37 | 0.2 |
| 153 | 37 | 0.6 |
| 154 | 37 | 0.4 |
| 155 | 37 | 0.4 |
| 156 | 37 | 0.2 |

For the DNA aptamers described below all the nucleotides (A, T, C and G) are deoxy. Unless noted otherwise, the individual sequences are represented in the 5′ to 3′ orientation. In some embodiments, the invention comprises aptamers with a nucleic acid sequences as described in Table 16 below. In other embodiments, the nucleic acid sequences of the aptamers described in Table 16 additionally comprise a 3′ cap (e.g., a 3′ inverted dT (3T)), and/or a 5′ amine (NH₂) modification to facilitate chemical coupling, and/or conjugation to a high molecular weight, non-immunogenic compound (e.g., PEG).

TABLE 16

DNA h-IgE Doped re-selection aptamer
sequence information (Clone sequences are
listed without the primer regions)

SEQ ID NO 113 D17.4
GGGGCACGTTTATCCGTCCCTCCTAGTGGCGTGCCCC
SEQ ID NO 119
GGGGCACATTTATCCGTCCCTCCTAGTGGTGTGCCCC
SEQ ID NO 120
GGGGTACCTTTATCCGTCCCTCCTAGTGGGGTGCCCC
SEQ ID NO 121
GGGGTACCTTTATCCGTCCCTCCTAGTGGGGTACCCC
SEQ ID NO 122
GGGGCAAATTTATCCGTCCCTCCTAGTGGTTTGCCCC
SEQ ID NO 123
GGGGCATATTTATCCGTCCCTCCTAGTGGTATGCCCC
SEQ ID NO 124
GGGGCACATTTATCCGTTCCTCCTAGTGGTGTGCCCC
SEQ ID NO 125
GGGGTACATTTATCCGTCCCTCCTAGTGGCATGCCCC
SEQ ID NO 126
GGGGCATGTTTATCCGTCCCTCCTAGTGGCATGCCCC
SEQ ID NO 127
GGGGCAACTTTATCCGTTCCTCCTAGTGGGTTGCCC
SEQ IDNO 128
GGGGCACATTCATCCGTCCCTCCTAGTGGTGTGCTCC
SEQ IDNO 129
GGGGTACCTTGATCCGTCCCTCCTAGTGGGGTGCCCC
SEQ ID NO 130
GGGGCATGTTTATCCGTTCCTCCTAGTGGCATGCCCC
SEQ ID NO 131
GGGGCAGCTTTATCCGTTCCTCCTAGTGGGCTGCCTC
SEQ ID NO 132
GGGGTACCTTTATCCGTTTCTCCTAGTGGGGTGCCCC
SEQ ID NO 133
GGGGTATGTTGATCCGTCCCTCCTAGTGGCATGCCCC
SEQ ID NO 134
GGGGCATGTTCATCCGTTCCTCCTAGTGGCGTGCCCC
SEQ ID NO 135
GGGACACATTTATCCGTTACTCTTAGTGGTGTGCCCC
SEQ ID NO 136
GGGGCACATTTATCCGTTACTCTTAGTGGTGTGCCCC
SEQ ID NO 137
GGGGCACGTTTACAGTCCCTCCTTATCGCCTCCC
SEQ ID NO 138
GGGGCACGTTTACAGTCCCTCCTTATCGCCTCCC
SEQ ID NO 139
GGGCAACTTTATCCGTTCCTCTTAGTGGGTTGCCCC
SEQ ID NO 140
GGGCTACTTTATCCGTCCCTCCTAGTGGGTAGCCCC
SEQ ID NO 141
GGCACCTTTATCCGTCCCTCCTAGTGGGGTGCCCC
SEQ ID NO 142
GGGGCACCTTTATCCGTCCCTCCTAGTGGGGTGCCCC
SEQ ID NO 143
GGGCACATTCATCCGTTCCTCCTAGTGGTGTGCCCC
SEQ ID NO 144
GGCACCTTTATCCGTTCCTTCTAGTGGGGTGCCC
SEQ ID NO 145
CGGCACCTTTATCCGTTACTCTTAGTGNGGTGCCCC
SEQ ID NO 146
GGCACCTTGATCCGTTCCTCCTAGTGGGGTGCCCC
SEQ ID NO 147
GCGGGCAAATTCATCCGTCCCTCCTAGTGGTTTGCCC
SEQ ID NO 148
GGGCACTTTATCCGTTCCTTCTAGTGGGTGTCCC
SEQ ID NO 149
GGCGGCAGCTTTATCCGTACCTCCCAGTGGGCTGCTCC
SEQ ID NO 150 ARC474
GGGGCAGCTTTATCCGTACCTCCCAGTGGGCTGCCCC
SEQ ID NO 151 ARC475
GGGGCTACTTTATCCGTCCCTCCTAGTGGGTAGCCCC
SEQ ID NO 152 ARC476
GGGGCTACTTTATCCGTACCTCCCAGTGGGTAGCCCC
SEQ ID NO 153 ARC477
GGGGCTACTTGATCCGTCCCTCCTAGTGGGTAGCCCC
SEQ ID NO 154 ARC478
GGGGCTACTTCATCCGTCCCTCCTAGTGGGTAGCCCC
SEQ ID NO 155 ARC479
GGGGCTACTTTATCCGTTCCTCTTAGTGGGTAGCCCC
SEQ ID NO 156 ARC480
GGGGCTACTTTATCCGTTCCTCCTAGTGGGTAGCCCC
SEQ ID NO 157 ARC656
GGGGCTACTTTATCCGTTCCTCCTAGTGGGTAGCCCC-3T

### Example 2E

### Aptamer Medicinal Chemistry of ARC445 for Increased Plasma Stability and Increased In Vitro Affinity

A highly stable and potent variant of ARC445 (SEQ ID NO 101) was identified through a systematic synthetic approach involving multiple phases of aptamer synthesis, purification and assaying for binding activity. Modifications, such as systematic replacement of the 2′-deoxy containing residues with 2′-O methyl containing residues was the basic approach used to achieve a significant increase in plasma nuclease resistance and overall stability.

During the processes of clone screening and minimization that led to ARC445 (SEQ ID NO 101), there was excellent agreement among the relative potency of aptamers in binding (as measured by dot-blot assay previously described), ELISA, FACS and histamine release assays (described in Example 3 below). Accordingly, the majority of test variants were tested for h-IgE binding affinity in dot-blot binding assays as an indicator of relative potency. For K_{D} determination, chemically synthesized aptamers were purified using denaturing polyacrylamide gel electrophoresis, 5′ end labeled with γ-³²P ATP and were tested for direct binding to full human h-IgE. An 8 point protein titration was used in the previously described dot blot binding assay (either {100 nM, 30 nM, 10 nM, 3 nM, 1 nM, 300 pM, 100 pM, 0 pM} or {10 nM, 3 nM, 1 nM, 300 pM, 100 pM, 30 pM, 10 pM, 0 pM}) in Dulbecco's PBS (with Mg⁺⁺ and Ca⁺⁺) with 0.1 mg/mL BSA at room temperature, for 30 minutes. K_{D} values were calculated by fitting the equation y=(max/(1+K/protein))+yint using KaleidaGraph (KaleidaGraph v. 3.51, Synergy Software). Sequences of the ARC445 derivatives synthesized, purified and assayed for binding to h-IgE as well as the results of the protein binding characterization are tabulated below in Table 17.

The first step in the process of replacing deoxy containing residues with 2′-O methyl containing residues was the synthesis and assay for binding activity of ARC1250-ARC1264 (SEQ ID NOs 158-172) each of which is equivalent to ARC445 with the addition of a 3′-inverted-dT (3T) and the replacement of a single 2′-deoxy residue with a 2′-O methyl residue. As can be seen from the binding data in Table 17, some positions readily tolerate substitution of a deoxy residue for a 2′-O methyl residue, while others do not. Interestingly, replacement of a 2′-deoxy residue with a 2′-O methyl residue conferred a significant improvement in affinity at position 10 (ARC1256) (SEQ ID NO 164).

Based upon the structure activity relationship (SAR) results from Phase I of the aptamer medicinal chemistry process, a second series of aptamers were designed, synthesized, purified and tested for binding to h-IgE. For these and all subsequent molecules, molecules that retained an affinity (K_{D}) of <1M or better where used in arriving at further aptamer design strategies. In Phase 2 which resulted in ARC1332-ARC1337 (SEQ ID NOs 173-178), the data from phase 1 was used to design more highly modified composite molecules using exclusively 2′-O methyl substitutions. The addition of stabilizing phosphorothioate containing linkages were also tested. The best of these in terms of simple binding affinity was ARC1335 (SEQ ID NO 176).

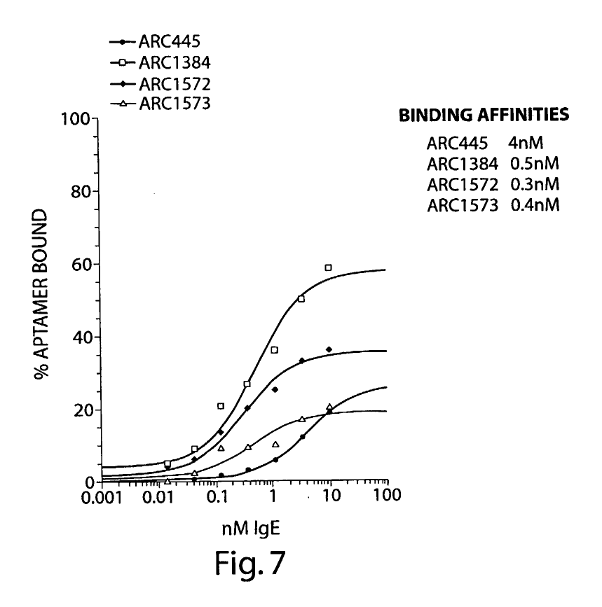

In Phases 3 (ARC1382-ARC1384, SEQ ID NOs 179-181) and Phase 4 of aptamer medicinal chemistry process (ARC1572-1573, SEQ ID NOs 182-183) the effects of further phosphorothioate modifications in the ARC1335 (SEQ ID NO 176) context were tested. The molecule from these limited series that seemed to strike the best balance between an intermediate number of phosphorothioates and highest affinity was ARC1384 (SEQ ID NO 181) (see FIG. 7).

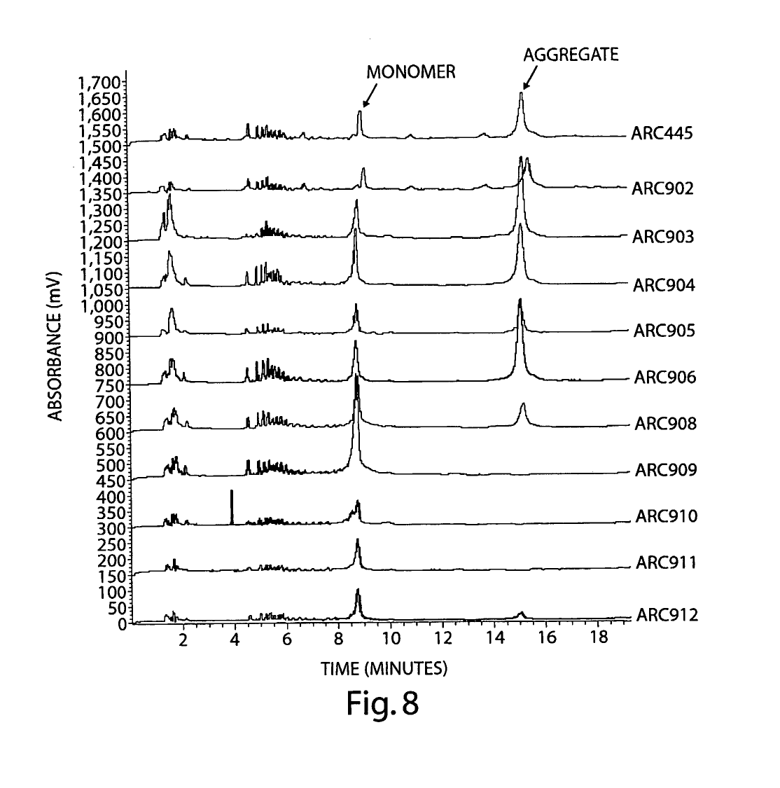

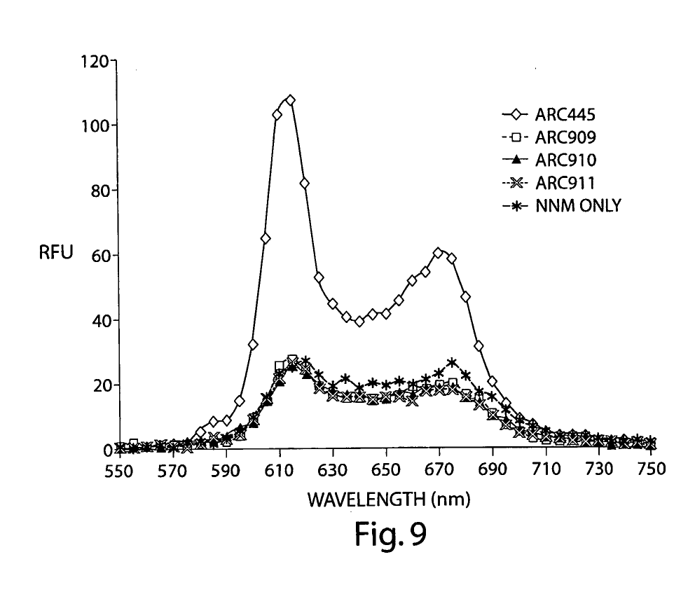

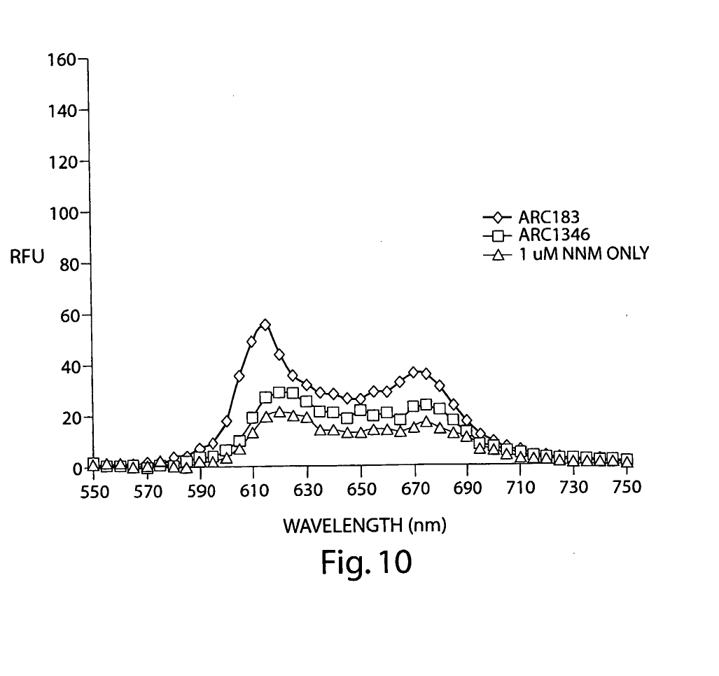

During the synthesis of ARC445 and its derivatives, peaks were observed on ion exchange HPLC that appeared to correspond to multimeric aggregates of ARC445. FIG. 8 is an example of the HPLC trace analysis of ARC445 and several derivatives which shows the multiple peaks due to aptamer aggregation. ARC445 contains 2 runs of guanosine residues at positions 6-9 and 16-21. While not intending to be bound by theory, either or both of these runs may be the cause of aptamer aggregation. It has been reported that in much the same way that ethidium bromide fluorescence is increased upon binding to duplex RNA and DNA, that N-methylmesoporphyrin IX (NMM) fluorescence is increased upon binding to G-quartet structures (Arthanari et al., Nucleic Acids Research, 26(16): 3724 (1996); Marathais et al., Nucleic Acids Research, 28(9): 1969 (2000); Joyce et al., Applied Spectroscopy, 58(7): 831 (2004)). Thus as shown in FIG. 9, we used NMM fluorescence to establish that ARC445 does in fact adopt a G-quartet structure. According to the literature protocols, 100 microliter reactions containing ˜1 micromolar NMM and 2 micromolar aptamer in Dulbecco's PBS containing magnesium and calcium were analyzed using a SpectraMax Gemini XS fluorescence plate reader. Fluorescence emission spectra were collected from 550 to 750 nm with and excitation wavelength of 405 nm. The G-quartet structure of the anti-thrombin DNA aptamer ARC183 has long been established in the literature (Macaya et al., Proc. Natl. Acad. Sci., 90: 3745 (1993)) and thus it was used as a positive control in this experiment (FIG. 10). ARC1346 shown in FIG. 10 is an aptamer of a similar size and nucleotide composition as ARC445 that is not predicted to have a C-quartet structure and is therefore a negative control in the experiment (FIG. 10). As can be seen FIGS. 9 and 10, ARC183 and ARC445 show a significant increase in NMM fluorescence relative to NMM alone while the negative control, ARC1346 does not.

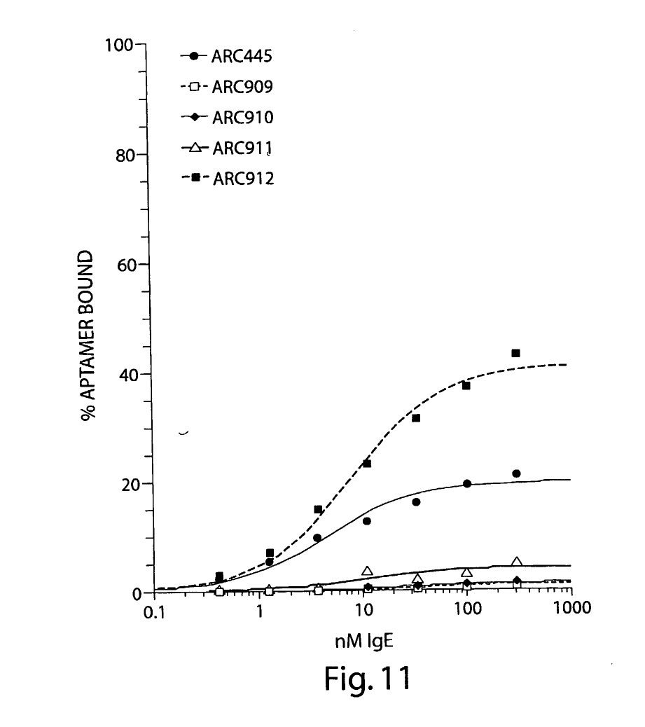

In addition to substituting dG with dG analogs and screening for minimization of the “C-quartet” signal in the NMM fluorescence assay, aptamers were screened for binding using the dot blot assay and the binding reaction conditions previously described (Dulbeccos PBS (with Ca⁺⁺ and Mg⁺⁺) plus 0.1 mg/mL BSA, room temperature for 30 minutes) to test whether dG analogs improved affinity for and thus potency against h-IgE. The first analog tested was deoxy-7-deaza G. With the nitrogen at position 7 on the purine base replaced by carbon, the hydrogen bond acceptor required for the G:G pairing in a G-quartet is effectively removed. In Phase 5.1 (SEQ ID NOs 184-201) each individual G in ARC445 is replaced by 7-deaza G. As can be seen in FIG. 8 and FIG. 9, removal of the G N-7 at positions 18-20 (ARC909-911, SEQ ID NOs 191-193) completely removed the aptamer aggregation observed by HPLC and NMM fluorescence. However these substitutions also completely remove binding to h-IgE, as demonstrated by the binding curves depicted in FIG. 11 (see also K_{D} values reported in Table 17). ARC912 (SEQ ID NO 194) also showed significant reduction in HPLC aggregation and retained some binding to h-IgE. Based upon the binding results and HPLC for ARC912 (SEQ ID NO 194), the mC:dG pair at position 3:21 was substituted for another Watson/Crick pairing that did not have a dG at position 21, in attempts to reduce multimeric aggregation. This was done in ARC1244-ARC1249 (SEQ ID NOs 195-200) but did not yield any aptamers with affinities even comparable to ARC445 (SEQ ID NO 101) and was thus dropped as an option for modification of ARC445.

While not wishing to be bound by any theory, substitution of dG with 7-deaza-G in provided the insight that it is the longer G-run from positions 16 to 21 that is the likely cause of the aggregation phenomenon and that the N-7 position in many of the residues tested may be required for high affinity binding to h-IgE either through direct hydrogen bonding interactions or through interactions with other residues in the aptamer itself that promote functional folding of the aptamer.

In phase 5.2 of the aptamer medicinal chemistry process (SEQ ID NOs 201-211), dG was substituted with deoxy inosine in the context of ARC1335 (SEQ ID NO 176). Since deoxy inosine lacks the exocyclic amine found in deoxy guanosine, a single amino to N7 hydrogen bond is removed from a potential G-quartet for each dG to dI substitution. Binding assays for ARCs 1548, 1552-1555, and 1562-1567 (SEQ ID NOs 201-211 respectively) revealed an SAR relationship for dG to dI substitution almost completely reversed from that of the dG to 7-deaza-G substitutions. In the dI series, substitutions were not tolerated at positions 6-9 while they were tolerated from moderately to very well at positions 16-21.

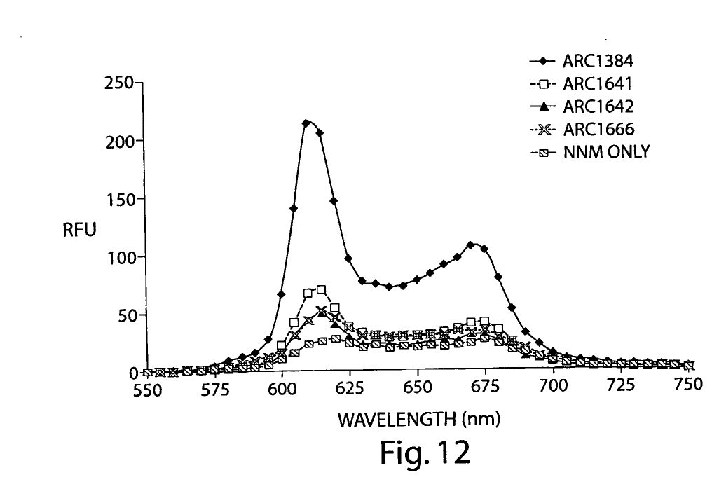

The results from phase 5.2 led to the design of composite molecules containing multiple dG to dI substitutions in the ARC1384 context. The results from phase 5.3 (SEQ ID NOs 212-219) yielded a number of aptamers with greatly improved affinity relative to ARC445 (SEQ ID NO 101) and ARC11384 (SEQ ID NO 181). For example, ARC1666 (SEQ ID NO 216) also showed significantly reduced G-quartet formation as assayed by NMM fluorescence, as depicted in FIG. 12.

In some embodiments, the invention comprises aptamers with a nucleic acid sequences as described in Table 17 below. In some embodiments, the nucleic acid sequences of the aptamers described in Table 17, where lacking, additionally comprise a 3′ cap (e.g., an inverted dT cap (3T)), and/or 5′ amine (NH₂) modification to facilitate chemical coupling, and/or conjugation to a high molecular weight, non-immunogenic compound (e.g., PEG). In other embodiments, the nucleic acid sequences described in Table 17 lack the indicated 3′ cap (e.g., a 3′ inverted dT cap (3T)). Lower case letters “m”, and “d” denote 2-O-methyl, and deoxy modifications respectively, “s” denotes an internucleotide phopshorothioate substitution, and “I” denotes an inosine substitution for guanosine.

TABLE 17

Sequences and Binding Affinities of ARC445
Modified Derivatives

| | | | |
|---|---|---|---|
| | | Sequence (5′->3′), | |
| | | (3T = inv dT), (T = dT), | |
| | | (s = phosphorothioate), | |
| | | (nN = 2′-O Methyl | |
| | | containing residue)(dI = | |
| SEQ | | deoxy inosine containing | |
| ID | | residue)(X = 7-deaza | K_{D} |
| NO | ARC # | guanosine containing residue | (nM) |

| | | | |
|---|---|---|---|
| 101 | ARC445 | sAdGmCmCmUdGdGdGdGdAmCmCmC | 3 |
| | | dAmUdGdGdGdGdGdGmCmU | |
| 158 | ARC1250 | mAdGmCmCmUdGdGdGdGdAmCmCmC | 11 |
| | | dAmUdGdGdGdGdGdGmCmU-3T | |
| 159 | ARC1251 | dAmGmCmCmUdGdGdGdGdAmCmCmC | 7 |
| | | dAmUdGdGdGdGdGdGmCmU-3T | |
| 160 | ARC1252 | dAdGmCmCmUmGdGdGdGdAmCmCmC | 98 |
| 161 | ARC1253 | dAdGmCmCmUdGmGdGdGdAmCmCmC | 2 |
| | | dAmUdGdGdGdGdGdGmCmU-3T | |
| 162 | ARC1254 | dAdGmCmCmUdGdGmGdGdAmCmCmC | 24 |
| | | dAmUdGdGdGdGdGdGmCmU-3T | |
| 163 | ARC1255 | dAdGmCmCmUdGdGdGmGdAmCmCmC | 8 |
| | | dAmUdGdGdGdGdGdGmCmU-3T | |
| 164 | ARC1256 | dAdGmCmCmUdGdGdGdGmAmCmCmC | 0.4 |
| | | dAmUdGdGdGdGdGdGmCmU-3T | |
| 165 | ARC1257 | dAdGmCmCmUdGdGdGdGdAmCmCmC | 6 |
| | | mAmUdGdGdGdGdGdGmCmU-3T | |
| 166 | ARC1258 | dAdGmCmCmUdGdGdGdGdAmCmCmC | 15 |
| | | dAmUmGdGdGdGdGdGmCmU-3T | |
| 167 | ARC1259 | dAdGmCmCmUdGdGdGdGdAmCmCmC | 5 |
| | | dAmUdGmGdGdGdGdGmCmU-3T | |
| 168 | ARC1260 | dAdGmCmCmUdGdGdGdGdAmCmCmC | 102 |
| | | dAmUdGdGmGdGdGdGmCmU-3T | |
| 169 | ARC1261 | dAdGmCmCmUdGdGdGdGdAmCmCmC | 63 |
| | | dAmUdGdGdGmGdGdGmCmU-3T | |
| 170 | ARC1262 | dAdGmCmCmUdGdGdGdGdAmCmCmC | 32 |
| | | dAmUdGdGdGdGmGdGmCmU-3T | |
| 171 | ARC1263 | dAdGmCmCmUdGdGdGdGdAmCmCmC | 64 |
| | | dAmUdGdGdGdGdGmGmCmU-3T | |
| 172 | ARC1264 | mAmGmCmCmUdGdGdGdGdAmCmCmC | 47 |
| | | dAmUdGdGdGdGmGmGmCmU-3T | |
| 173 | ARC1332 | dAmGmCmCmUdGdGdGdGmAmCmCmC | 0.5 |
| | | mAmUdGdGdGdGdGdGmCmU-3T | |
| 174 | ARC1333 | dAmGmCmCmUdGmGdGdGdAmCmCmC | 1.5 |
| | | dAmUdGmGdGdGdGdGmCmU-3T | |
| 175 | ARC1334 | dAmGmCmCmUdGmGdGdGmAmCmCmC | 0.3 |
| | | mAmUdGmGdGdGdGdGmCmU-3T | |
| 176 | ARC1335 | mAmGmCmCmUdGmGdGdGmAmCmCmC | 0.3 |
| | | mAmUdGmGdGdGdGdGmCmU-3T | |
| 177 | ARC1336 | dA-s-mGmCmCmUdGmGdGdGmAmCm | No |
| | | CmCmAmU-s-mGdGdGdG-s-dGmCm | Binding |
| | | U-3T | |
| 178 | ARC1337 | dA-s-mGmCmCmUdGmGdGdG-s-mA | 0.2 |
| | | mCmCmCmAmU-s-dG-s-mGdGdGd | |
| | | G-s-dGmCmU-3T | |
| 179 | ARC1382 | mAmGmCmCmUdGmGdG-s-dGmAmCm | 1.1 |
| | | CmCmAmU-s-dG-s-mGdG-s-dGmC | |
| | | mU-3T | |
| 180 | ARC1383 | mAmGmCmCmUdGmGdG-s-dGmAmCm | 1 |
| | | CmCmAmU-s-dGmGdG-s-dGdG-s- | |
| | | dGmCmU-3T | |
| 181 | ARC1384 | mAmGmCmCmUdGmG-s-dGmAmCmCm | 0.5 |
| | | CmAmU-s-dG-s-mGdG-s-dGdG- | |
| | | s-dGmCmU-3T | |
| 182 | ARC1572 | mAmGmCmCmU-s-dGmG-s-dG-s-d | 0.3 |
| | | GmAmCmCmAmU-s-dGmG-s-dG-s- | |
| | | dG-s-dG-s-dGmCmU-3T | |
| 183 | ARC1573 | mAmGmCmCmU-s-dG-s-mG-s-dG- | 0.4 |
| | | s-dGmAmCmCmCmAmU-s-dG-s-m | |
| | | G-s-dG-s-dG-s-dG-s-dG-s-mC | |
| | | mU-3T | |
| 184 | ARC902 | dAXmCmCmUdGdGdGdGdAmCmCmCd | 9 |
| | | AmUdGdGdGdGdGdGmCmU | |
| 185 | ARC903 | dAdGmCmCmUXdGdGdGdAmCmCmCd | 14 |
| | | AmUdGdGdGdGdGdGmCmU | |
| 186 | ARC904 | dAdGmCmCmUdGXdGdGdAmCmCmCd | 8 |
| | | AmUdGdGdGdGdGdGmCmU | |
| 187 | ARC905 | dAdGmCmCmUdGdGXdGdAmCmCmCd | 9 |
| | | AmUdGdGdGdGdGdGmCmU | |
| 188 | ARC906 | dAdGmCmCmUdGdGdGXdAmCmCmCd | 88 |
| | | AmUdGdGdGdGdGdGmCmU | |
| 189 | ARC907 | dAdGmCmCmUdGdGdGdGdAmCmCmC | 22 |
| | | dAmUXdGdGdGdGdGmCmU | |
| 190 | ARC908 | dAdGmCmCmUdGdGdGdGdAmCmCmC | 3 |
| | | dAmUdGXdGdGdGdGmCmU | |
| 191 | ARC909 | dAdGmCmCmUdGdGdGdGdAmCmCmC | No |
| | | dAmUdGdGXdGdGdGmCmU | Binding |
| 192 | ARC910 | dAdGmCmCmUdGdGdGdGdAmCmCmC | No |
| | | dAmUdGdGdGXdGdGmCmU | Binding |
| 193 | ARC911 | dAdGmCmCmUdGdGdGdGdAmCmCmC | No |
| | | dAmUdGdGdGdGXdGmCmU | Binding |
| 194 | ARC912 | dAdGmCmCmUdGdGdGdGdAmCmCmC | 9 |
| | | dAmUdGdGdGdGdGXmCmU | |
| 195 | ARC1244 | dAdGdGmCmUdGdGdGdGdAmCmCmC | 450 |
| | | dAmUdGdGdGdGdGmCmCmU | |
| 196 | ARC1245 | dAdGmGmCmUdGdGdGdGdAmCmCmC | 198 |
| | | dAmUdGdGdGdGdGmCmCmU | |
| 197 | ARC1246 | dAdGmUmCmUdGdGdGdGdAmCmCmC | 34 |
| | | dAmUdGdGdGdGdGmAmCmU | |
| 198 | ARC1247 | dAdGmUmCmUdGdGdGdGdAmCmCmC | 192 |
| | | dAmUdGdGdGdGdGmAmCmU | |
| 199 | ARC1248 | dAdGdAmCmUdGdGdGdGdAmCmCmC | 38 |
| | | dAmUdGdGdGdGdGmAmCmU | |
| 200 | ARC1249 | dAdGmAmCmUdGdGdGdGdAmCmCmC | 130 |
| | | dAmUdGdGdGdGdGmAmCmU | |
| 201 | ARC1548 | mAdImCmCmUdGmGdGdGmAmCmCmC | 3.3 |
| | | mAmUdGmGdGdGdGdGmCmU-3T | |
| 202 | ARC1552 | mAmGmCmCmUdImGdGdGmAmCmCmC | No |
| | | mAmUdGmGdGdGdGdGmCmU-3T | Binding |
| 203 | ARC1553 | mAmGmCmCmUdGdIdGdGmAmCmCmC | No |
| | | mAmUdGmGdGdGdGdGmCmU-3T | Binding |
| 204 | ARC1554 | mAmGmCmCmUdGmGdIdGmAmCmCmC | No |
| | | mAmUdGmGdGdGdGdGmCmU-3T | Binding |
| 205 | ARC1555 | mAmGmCmCmUdGmGdGdImAmCmCmC | No |
| | | mAmUdGmGdGdGdGdGmCmU-3T | Binding |
| 206 | ARC1562 | mAmGmCmCmUdGmGdGdGmAmCmCmC | 0.6 |
| | | mAmUdIdGdGdGdGdGmCmU-3T | |
| 207 | ARC1563 | mAmGmCmCmUdGmGdGdGmAmCmCmC | 2.2 |
| | | mAmUdGdIdGdGdGdGmCmU-3T | |
| 208 | ARC1564 | mAmGmCmCmUdGmGdGdGmAmCmCmC | 0.5 |
| | | mAmUdGmGdIdGdGdGmCmU-3T | |
| 209 | ARC1565 | mAmGmCmCmUdGmGdGdGmAmCmCmC | 1.4 |
| | | mAmUdGmGdGdIdGdGmCmU-3T | |
| 210 | ARC1566 | mAmGmCmCmUdGmGdGdGmAmCmCmC | 0.1 |
| | | mAmUdGmGdGdGdIdGmCmU-3T | |
| 211 | ARC1567 | mAmGmCmCmUdGmGdGdGmAmCmCmC | 3.5 |
| | | mAmUdGmGdGdGdGdImCmU-3T | |
| 212 | ARC1641 | mAmGmCmCmUdGmGdG-s-dGmAmCm | 0.4 |
| | | CmCmAmU-s-dG-s-mGdG-s-dGd | |
| | | I-s-dGmCmU-3T | |
| 213 | ARC1642 | mAmGmCmCmUdGmGdG-s-dGmAmCm | 0.4 |
| | | CmCmAmU-s-dI-s-mGdG-s-dGd | |
| | | I-s-dGmCmU-3T | |
| 214 | ARC1643 | mAmGmCmCmU-s-dGmG-s-dG-s-d | 1.1 |
| | | GmAmCmCmCmAmU-s-dGmG-s-dG- | |
| | | s-dG-s-dI-s-dGmCmU-3T | |
| 215 | ARC1644 | mAmGmCmCmU-s-dGmG-s-dG-s-d | 1.7 |
| | | GmAmCmCmCmAmU-s-dImG-s-dG- | |
| | | s-dG-s-dI-s-dGmCmU-3T | |
| 216 | ARC1666 | mAmGmCmCmUdGmGdG-s-dGmAmCm | 0.035 |
| | | CmCmAmU-s-dI-s-mGdI-s-dGd | |
| | | I-s-dGmCmU-3T | |
| 217 | ARC1667 | mAmGmCmCmUdGmGdG-s-dGmAmCm | 0.4 |
| | | CmCmAmU-s-dG-s-mGdI-s-dGd | |
| | | I-s-dGmCmU-3T | |
| 218 | ARC1728 | mAmGmCmCmU-s-dGmG-s-dG-s-d | 2.3 |
| | | GmAmCmCmCmAmU-s-dGmG-s-dI- | |
| | | s-dGmCmU-3T | |
| 219 | ARC1729 | mAmGmCmCmU-s-dGmG-s-dG-s-d | 1 |
| | | GmAmCmCmCmAmU-s-dImG-s-dI- | |
| | | s-dG-s-dI-s-dGmCmU-3T | |

### Example 2F

### ARC656 Aptamer Medicinal Chemistry

A highly stable and potent variant of ARC656 (SEQ ID NO 157) (described in Example 2D) was identified through a systematic synthetic approach involving multiple phases of aptamer synthesis, purification and assay for binding activity. Systematic replacement of the 2′-deoxy containing residues with 2′-O methyl containing residues was the basic approach used, in order to achieve a significant increase in plasma nuclease resistance and overall stability.

During the processes of clone screening and minimization that led to ARC656 (SEQ ID NO 157), there was excellent agreement among the relative potency of aptamers in binding (as measured by dot-blot assay previously described), ELISA, FACS and histamine release assays (described in Example 3 below). Accordingly the test variant binding affinity for h-IgE measured in a dot-blot assay binding assay was used to characterize the relative potency of the majority of the aptamers synthesized. For K_{D} determination, chemically synthesized aptamers were purified using denaturing polyacrylamide gel electrophoresis, 5′ end labeled with γ-³²P ATP and were tested for direct binding to full human IgE. An 8 point protein titration was used in the dot blot binding assay {30 nM, 10 nM, 3 nM, 1 nM, 300 pM, 100 pM, 30 pM 0 pM} using Dulbecco's PBS (with Mg⁺⁺ and Ca⁺⁺) with 0.1 mg/mL BSA. K_{D} values were calculated by fitting the equation y=(max/(1+K/protein))+yint using KaleidaGraph (KaleidaGraph v. 3.51, Synergy Software). Sequences of the ARC656 derivatives synthesized, purified and assayed for binding to h-IgE as well as the results of the protein binding characterization are tabulated below in Table 18.

The first step in the process of replacing deoxy containing residues with 2′-O methyl containing residues was the synthesis and assay for binding activity of ARC1265-ARC1306 (SEQ ID NOs 220-261), each of which is equivalent to ARC656 with the replacement of a single 2′-deoxy residue with a 2′-O methyl residue. As can be seen from the binding data in Table 18, some positions readily tolerate substitution of a deoxy residue for a 2′-O methyl residue, while others do not. The proposed stem region of ARC656 best tolerated substitution of deoxy containing residues with 2′-O methyl residues. In addition, phase one included some block substitutions of deoxy to 2′-O methyl in the stem region of the aptamer.

Based upon the structure activity relationship (SAR) results from Phase 1 of the aptamer medicinal chemistry design, a second series of aptamers were designed, synthesized, purified and tested for binding to h-IgE. The addition of stabilizing phosphorothioate containing linkages (ARC1391 (SEQ ID NO 266) & ARC1417 (SEQ ID NO 292)) were also tested. The best of these in terms of simple binding affinity was ARC1410 (SEQ ID NO 285). While it is clear that many of the constructs tested retain relatively high affinity binding for h-IgE, few of them retain affinity equal to the parent compound ARC656 (SEQ ID NO 157).

In some embodiments, the invention comprises aptamers with a nucleic acid sequences as described in Table 18 below. In some embodiments, the nucleic acid sequences of the aptamers described in Table 18, where lacking, additionally comprise a 3′ cap (e.g., an inverted dT cap (3T)), and/or 5′ amine (NH₂) modification to facilitate chemical coupling, and/or conjugation to a high molecular weight, non-immunogenic compound (e.g., PEG). In other embodiments, the nucleic acid sequences described in Table 18 lack the indicated 3′ cap (e.g. a 3′ inverted dT cap (3T)). Lower case letters “m”, and “d” preceding nucleotide abbreviations A, C, G, or T denote 2-O-methyl, and deoxy modifications respectively, and “s” denotes an internucleotide phosphorothioate substitution.

TABLE 18

Sequences and Binding Affinities of ARC656
Modified Derivatives

| | | | |
|---|---|---|---|
| | | Sequence (5′->3′), | |
| | | (3T = inv dT), (T = dT), | |
| | | (s = phosphorothioate), | |
| | | (nN = 2′-O Methyl | |
| | | containing residue)(dI = | |
| SEQ | | deoxy inosine containing | |
| ID | | residue)(X = 7-deaza | K_{D} |
| NO | ARC # | guanosine containing residue | (nM) |

| | | | |
|---|---|---|---|
| 157 | ARC656 | dGdGdGdGdCTdAdCTTTdATdCdCdGT | 0.2 |
| | | TdCdCTdAdGTdGdGdGTdAdGdCdCdC | |
| | | dC-3T | |
| 220 | ARC1265 | mGdGdGdGdCTdAdCTTTdATdCdCdGT | 0.2 |
| | | TdCdCTdCdCTdAdGTdGdGdGTdAdGd | |
| | | CdCdCdC-3T | |
| 221 | ARC1266 | dGmGdGdGdCTdAdCTTTdATdCdCdGT | 0.2 |
| | | TdCdCTdCdCTdAdGTdGdGdGTdAdGd | |
| | | CdCdCdC-3T | |
| 222 | ARC1267 | dGdGmGdGdCTdAdCTTTdATdCdCdGT | 0.1 |
| | | TdCdCTdCdCTdAdGTdGdGdGTdAdGd | |
| | | CdCdCdC-3T | |
| 223 | ARC1268 | dGdGdGmGdCTdAdCTTTdATdCdCdGT | 0.2 |
| | | TdCdCTdCdCTdAdGTdGdGdGTdAdGd | |
| | | CdCdCdC-3T | |
| 224 | ARC1269 | dGdGdGdGmCTdAdCTTTdATdCdCdGT | 0.3 |
| | | TdCdCTdCdCTdAdGTdGdGdGTdAdGd | |
| | | CdCdCdC-3T | |
| 225 | ARC1270 | dGdGdGdGdCmUdAdCTTTdATdCdCdG | 0.2 |
| | | TTdCdCTdCdCTdAdGTdGdGdGTdAdG | |
| | | dCdCdCdC-3T | |
| 226 | ARC1271 | dGdGdGdGdCTmAdCTTTdATdCdCdGT | 0.2 |
| | | TdCdCTdCdCTdAdGTdGdGdGTdAdGd | |
| | | CdCdCdC-3T | |
| 227 | ARC1272 | dGdGdGdGdCTdAmCTTTdATdCdCdGT | 0.2 |
| | | TdCdCTdCdCTdAdGTdGdGdGTdAdGd | |
| | | CdCdCdC-3T | |
| 228 | ARC1273 | dGdGdGdGdCTdAdCmUTTdATdCdCdG | 3 |
| | | TTdCdCTdCdCTdAdGTdGdGdGTdAdG | |
| | | dCdCdCdC-3T | |
| 229 | ARC1274 | dGdGdGdGdCTdAdCTmUTdATdCdCdG | 2 |
| | | TTdCdCTdCdCTdAdGTdGdGdGTdAdG | |
| | | dCdCdCdC-3T | |
| 230 | ARC1275 | dGdGdGdGdCTdAdCTTmUdATdCdCdG | 0.3 |
| | | TTdCdCTdCdCTdAdGTdGdGdGTdAdG | |
| | | dCdCdCdC-3T | |
| 231 | ARC1276 | dGdGdGdGdCTdAdCTTTmATdCdCdGT | 3 |
| | | TdCdCTdCdCTdAdGTdGdGdGTdAdGd | |
| | | CdCdCdC-3T | |
| 232 | ARC1277 | dGdGdGdGdCTdAdCTTTdAmUdCdCdG | 6 |
| | | TTdCdCTdCdCTdAdGTdGdGdGTdAdG | |
| | | dCdCdCdC-3T | |
| 233 | ARC1278 | dGdGdGdGdCTdAdCTTTdATmCdCdGT | 2 |
| | | TdCdCTdCdCTdAdGTdGdGdGTdAdGd | |
| | | CdCdCdC-3T | |
| 234 | ARC1279 | dGdGdGdGdCTdAdCTTTdATdCmCdGT | 0.8 |
| | | TdCdCTdCdCTdAdGTdGdGdGTdAdGd | |
| | | CdCdCdC-3T | |
| 235 | ARC1280 | dGdGdGdGdCTdAdCTTTdATdCdCmGT | 0.6 |
| | | TdCdCTdCdCrdAdGTdGdGdGTdAdGd | |
| | | CdCdCdC-3T | |
| 236 | ARC1281 | dGdGdGdGdCTdAdCTTTdATdCdCdGm | 0.1 |
| | | UTdCdCTdAdGTdGdGdGTdAdGdCdCd | |
| | | CdC-3T | |
| 237 | ARC1282 | dGdGdGdGdCTdAdCTTTdATdCdCdGT | 0.2 |
| | | mUdCdCTdCdCTdAdGTdGdGdGTdAdG | |
| | | dCdCdCdC-3T | |
| 238 | ARC1283 | dGdGdGdGdCTdAdCTTTdATdCdCdGT | 0.6 |
| | | TmCdCTdCdCTdAdGTdGdGdGTdAdGd | |
| | | CdCdCdC-3T | |
| 239 | ARC1284 | dCdGdGdGdCTdAdCTTTdATdCdCdGT | 0.1 |
| | | TdCmCTdCdCTdAdGTdGdGdGTdAdGd | |
| | | CdCdCdC-3T | |
| 240 | ARC1285 | dGdGdGdGdCTdAdCTTTdATdCdCdGT | 0.8 |
| | | TdCdCmUdCdCTdAdGTdGdGdGTdAdG | |
| | | dCdCdCdC-3T | |
| 241 | ARC1286 | dGdGdGdGdCTdAdCTTTdATdCdCdGT | 7 |
| | | TdCdCTmCdCTdAdGTdGdGdGTdAdGd | |
| | | CdCdCdC-3T | |
| 242 | ARC1287 | dGdGdGdGdCTdAdCTTTdATdCdCdGT | 1.0 |
| | | TdCdCTdCmCTdAdGTdGdGdGTdAdGd | |
| | | CdCdCdC-3T | |
| 243 | ARC1288 | dGdGdGdGdCTdAdCTTTdATdCdCdGT | 2 |
| | | TdCdCTdCdCmUdAdGTdGdGdGTdAdG | |
| | | dCdCdCdC-3T | |
| 244 | ARC1289 | dGdGdGdGdCTdAdCTTTdATdCdCdGT | No |
| | | TdCdCTdCdCTmAdGTdGdGdGTdAdGd | Binding |
| | | CdCdCdC-3T | |
| 245 | ARC1290 | dGdGdGdGdCTdAdCTTTdATdCdCdGT | No |
| | | TdCdCTdCdCTdAmGTdGdGdGTdAdGd | Binding |
| | | CdCdCdC-3T | |
| 246 | ARC1291 | dGdGdGdGdCTdAdCTTTdATdCdCdGT | No |
| | | TdCdCTdCdCTdAdGmUdGdGdGTdAdG | Binding |
| | | dCdCdCdC-3T | |
| 247 | ARC1292 | dGdGdGdGdCTdAdCTTTdATdCdCdGT | 22 |
| | | TdCdCTdCdCTdAdGTmGdGdGTdAdGd | |
| | | CdCdCdC-3T | |
| 248 | ARC1293 | dGdGdGdGdCTdAdCTTTdATdCdCdGT | No |
| | | TdCdCTdCdCTdAdGTGmGdGTdAdGdC | Binding |
| | | dCdCdC-3T | |
| 249 | ARC1294 | dGdGdGdGdCTdAdCTTTdATdCdCdGT | 8 |
| | | TdCdCTdCdCTdAdGTdGdGmGTdAdGd | |
| | | CdCdCdC-3T | |
| 250 | ARC1295 | dGdGdGdGdCTdAdCTTTdATdCdCdGT | 3 |
| | | TdCdCTdCdCTdAdGTdGdGdGmUdAdG | |
| | | dCdCdCdC-3T | |
| 251 | ARC1296 | dGdGdGdGdCTdAdCTTTdATdCdCdGT | 1.2 |
| | | TdCdCTdCdCTdAdGTdGdGdGTmAdGd | |
| | | CdCdCdC-3T | |
| 252 | ARC1297 | dGdGdGdGdCTdAdCTTTdATdCdCdGT | 0.8 |
| | | TdCdCTdCdCTdAdGTdGdGdGTdAmGd | |
| | | CdCdCdC-3T | |
| 253 | ARC1298 | dGdGdGdGdCTdAdCTTTdATdCdCdGT | 0.4 |
| | | TdCdCTdCdCTdAdGTdGdGdGTdAdGm | |
| | | CdCdCdC-3T | |
| 254 | ARC1299 | dGdGdGdGdCIdAdCTTTdATdCdCdGT | 0.5 |
| | | TdCdCTdCdCTdAdGTdGdGdGTdAdGd | |
| | | CmCdCdC-3T | |
| 255 | ARC1300 | dGdGdcdGdCTdAdcTTTdATdCdCdGT | 0.7 |
| | | TdCdCTdCdCTdAdGTdGdGdGTdAdGd | |
| | | GdCdCmCdC-3T | |
| 256 | ARC1301 | dGdGdGdGdCTdAdcTTTdATdCdCdGT | 0.5 |
| | | TdCdCTdCdCTdAdGTdGdGdGTdAdGd | |
| | | CdCdCmC-3T | |
| 257 | ARC1302 | mGmGmGmGdCTmAdCTTTdATdCdCdGT | 19 |
| | | TdCdCTdCdCTdAdGTdGdGmGTmAmGd | |
| | | CdCdCdCdC-3T | |
| 258 | ARC1303 | dGdGdGdGmCmUdAmCTTTdATdCdCdG | 1.3 |
| | | TTdCdCTdCdCTdAdCTdGdGdGmUdAd | |
| | | GmCmCmCmC-3T | |
| 259 | ARC1304 | mGmGmGmGdCTdAdCTTTdATdCdCdGT | 0.6 |
| | | TdCdCTdCdCTdAdGTdCdGdGTdAdGm | |
| | | CmCmCmC-3T | |
| 260 | ARC1305 | mGmGmGmGmCTdAdCTTTdATdCdCdGT | No |
| | | TdCdCTdCdCTdAdGTdCdGdGTdAmGm | Binding |
| | | CmCmCmC-3T | |
| 261 | ARC1306 | mGmGmGmCTdAdCTTTdATdCdCdGTTd | 14 |
| | | CdCTdCdCTdAdGTdGdGdGTdAMGmCm | |
| | | CmC-3T | |
| 262 | ARC1387 | mGmGmGmGdCTmAdCTTTdATdCdCdGT | 2.4 |
| | | TdCdCTdCdCTdAdGTdGdGdGTdAmGm | |
| | | CmCmCmC-3T | |
| 263 | ARC1388 | mGmGmGmGmCmUmAmCTTTdATdCdCdG | 4.4 |
| | | TTdCdCTdCdCTdAdGTdGdGdGTdAmG | |
| | | mCmCmCmC-3T | |
| 264 | ARC1389 | mGmGmGmGdCTmAdCTTmUdATdCdCdG | 5.4 |
| | | mUmUdCmCTdCdCTdAdGTdGdGdGTdA | |
| | | mGmCmCmCmC-3T | |
| 265 | ARC1390 | mGmGmGmGmCmUmAmCTTmUdATdCdCd | 17 |
| | | GmUmUdCmCTdCdCTdAdGTdGdGdGTd | |
| | | AmGmCmCmCmC-3T | |
| 266 | ARC1391 | mGmGmGmGdCTmAdC-s-TTTdATdCdC | 4.4 |
| | | dGTTdCdCTdCdCTdAdGTdGdGdGTdA | |
| | | mGmCmCmCmC-3T | |
| 267 | ARC1392 | mGmGmGmGdCTmAdCT-s-TTdATdCdC | 3 |
| | | dGTTdCdCTdCdCTdAdGTdGdGdGTdA | |
| | | mGmCmCmCmC-3T | |
| 268 | ARC1393 | mGmGmGmGdCTmAdCTT-s-TdATdCdC | No |
| | | dGTTdCdCTdCdCTdAdGTdGdGdGTdA | Binding |
| | | mGmCmCmCmC-3T | |
| 269 | ARC1394 | mGmGmGmGdCTmAdCTTT-s-dATdCdC | 8.1 |
| | | dGTTdCdCTdCdCTdAdGTdGdGdGTdA | |
| | | mGmCmCmCmC-3T | |
| 270 | ARC1395 | mGmGmGmGdCTmAdCTTTdA-s-TdCdC | 5.7 |
| | | dGTTdCdCTdCdCTdAdGTdGdGdGTdA | |
| | | mGmCmCmCmC-3T | |
| 271 | ARC1396 | mGmGmGmGdCTmAdCTTTdAT-s-dCdC | 2 |
| | | dGTTdCdCTdCdCTdAdGTdGdGdGTdA | |
| | | mGmCmCmCmC-3T | |
| 272 | ARC1397 | mGmGmGmGdCTmAdCTTTdATdC-s-dC | 1.8 |
| | | dGTTdCdCTdCdCTdAdGTdGdGdGTdA | |
| | | mGmCmCmCmC-3T | |
| 273 | ARC1398 | mGmGmGmGdCTmAdCTTTdATdCdC-s- | 2.7 |
| | | dGTTdCdCTdCdCTdAdGTdGdGdGTdA | |
| | | mGmCmCmCmC-3T | |
| 274 | ARC1399 | mGmGmGmGdCTmAdCTTTdATdCdCdG- | 1.8 |
| | | s-TTdCdCTdCdCTdAdGTdGdGdGTdA | |
| | | mGmCmCmCmC-3T | |
| 275 | ARC1400 | mGmGmGmGdCTmAdCTTTdATdCdCdG | 2.4 |
| | | T-s-TdCdCTdCdCTdAdGTdGdGdGT | |
| | | dAmGmCmCmCmC-3T | |
| 276 | ARC1401 | mGmGmGmGdCTmAdCTTTdATdCdCdGT | 2 |
| | | T-s-dCdCTdCdCTdAdGTdGdGdGTdA | |
| | | mGmCmCmCmC-3T | |
| 277 | ARC1402 | mGmGmGmGdCTmAdCTTTdATdCdCdGT | 2.1 |
| | | TdC-s-dCTdCdCTdAdGTdGdGdGTdA | |
| | | mGmCmCmCmC-3T | |
| 278 | ARC1403 | mGmGmGmGdCTmAdCTTTdATdCdCdGT | Not |
| | | TdC-s-TdCdCTdAdGTdGdGdGTdAmG | Done |
| | | mCmCmCmC-3T | |
| 279 | ARC1404 | mGmGmGmGdCTmAdCTTTdATdCdCdGT | 1.8 |
| | | TdCdCT-s-dCdCTdAdGTdGdGdGTdA | |
| | | mGmCmCmCmC-3T | |
| 280 | ARC1405 | mGmGmGmGdCTmAdCTTTdATdCdCdGT | 2.5 |
| | | TdCdCTdC-s-dCTdAdGTdGdGdGTdA | |
| | | mGmCmCmCmC-3T | |
| 281 | ARC1406 | mGmGmGmGdCTmAdCTTTdATdCdCdGT | 3.2 |
| | | TdCdCTdCdC-s-TdAdGTdGdGdGTdA | |
| | | mGmCmCmCmC-3T | |
| 282 | ARC1407 | mGmGmGmGdCTmAdCTTTdATdCdCdGT | 1.8 |
| | | TdCdCTdCdCT-s-dAdGTdGdGdGTdA | |
| | | mGmCmCmCmC-3T | |
| 283 | ARC1408 | mGmGmGmGdCTmAdcTTTdATdCdCdGT | 1 |
| | | TdCdCTdCdCTdA-s-dGTdGdGdGTdA | |
| | | mGmCmCmCmC-3T | |
| 284 | ARC1409 | mGmGmGmGdCTmAdCTTTdATdCdCdGT | Not |
| | | TdCdCTdCdCTdAdG-s-TdGdGdGTdA | Done |
| | | mGmCmCmCmC-3T | |
| 285 | ARC1410 | mGmGmGmGdCTmAdcTTTdATdCdCdGT | 0.7 |
| | | TdCdCTdCdCTdAdGT-s-dGdGdGTdA | |
| | | mGmCmCmCmC-3T | |
| 286 | ARC1411 | mGmGmGmGdCTmAdcTTTdATdCdCdGT | 5.4 |
| | | TdCdCTdCdCTdAdGTdG-s-dGdGTdA | |
| | | mGmCmCmCmC-3T | |
| 287 | ARC1412 | mGmGmGmGdCTmAdCTTTdATdCdCdGT | 2.5 |
| | | TdCdCTdCdCTdAdGTdGdG-s-dGTdA | |
| | | mGmCmCmCmC-3T | |
| 288 | ARC1413 | mGmGmGmGdCTmAdCTTTdATdCdCdGT | 3.3 |
| | | TdCdCTdCdCTdAdGTdGdGdG-s-TdA | |
| | | mGmCmCmCmC-3T | |
| 289 | ARC1414 | mGmGmGmGdCTmAdCTTTdATdCdCdGT | 2.9 |
| | | TdCdCTdCdCTdAdGTdGdGdGT-s-dA | |
| | | mGmCmCmCmC-3T | |
| 290 | ARC1415 | mGmGmGmGdCTmAdCT-s-TTdA-s-Td | 1.2 |
| | | C-s-dCdGTT-s-dCdCT-s-dCdCT- | |
| | | s-dAdGT-s-dGdGdG-s-TdAmGmCmC | |
| | | mCmC-3T | |
| 291 | ARC1416 | mGmGmGmGmCmUmAmCT-s-TTdA-s-T | No |
| | | dC-s-dCdGTT-s-dCdCT-s-dCdCT- | Binding |
| | | s-dAdGT-s-dGdGdG-s-TdAmGmCmC | |
| | | mCmC-3T | |
| 292 | ARC1417 | mGmGmGmGmCmUmAmCT-s-TmUdA-s- | 27 |
| | | TdC-s-dCdGmUmUdCmCT-s-dCdCT- | |
| | | s-dAdGT-s-dGdGdG-s-TdAmGmCmC | |
| | | mCmC-3T | |

### Example 2G

### ARC1666 Aptamer Medicinal Chemistry

Additional stabilized and potent variants of ARC1666 (SEQ ID NO 101) were identified through a systematic synthetic approach involving multiple phases of aptamer synthesis, purification and assaying for binding activity. A series of aptamers were designed in which additional stabilizing phosphorothioate containing linkages were introduced in the context of ARC1666. In Phase 1, multiple stabilizing phosphorothioate linkages were introduced at the 3′ end of ARC1666 in an attempt to increase serum stability by decreasing nuclease degradation at the 3′ end. In Phase 2, stabilizing phosphorothioate linkages were introduced at sites that do not contain phosphorothioate linkages in ARC1666. In Phase 3, additional 2′-OMe substitutions were introduced in the context of ARC1666 and the terminal stem was lengthened.

A majority of test variants were tested for h-IgE binding affinity in the dot-blot binding assays previously described as an indicator of relative potency. Chemically synthesized aptamers were purified using denaturing polyacrylamide gel electrophoresis, 5′ end labeled with γ-³²P ATP and were tested for direct binding to full length human h-IgE (Athens Research, Athens, Ga.). A protein titration was used in the dot blot binding assay in Dulbecco's PBS (with Mg⁺⁺ and Ca⁺⁺) with 0.1 mg/mL BSA at room temperature, for 30 minutes. K_{D} values were calculated by fitting the equation y=Max*((([aptamer]+[protein]+K_{D})−SQRT(([aptamer]+[protein]+K_{D})̂2−4([aptamer][protein])))/(2*[aptamer]))+y-Int.

Sequences of all ARC1666 derivatives synthesized, purified and assayed for binding to h-IgE as well as the results of the protein binding characterization are tabulated below in Table 19 below. As can be seen in Table 19, the binding affinities of the derivatives from Phase 1 and 2 were similar to that of ARC1666, indicating the introduction of additional phosphorothioate linkages did not have an adverse effect on binding affinity.

In some embodiments, the invention comprises aptamers with a nucleic acid sequences as described in Table 19 below. In some embodiments, the nucleic acid sequences of the aptamers described in Table 19, where lacking, additionally comprise a 3′ cap (e.g., an inverted dT cap (3T)), and/or 5′ amine (NH₂) modification to facilitate chemical coupling, and/or conjugation to a high molecular weight, non-immunogenic compound (e.g., PEG). In other embodiments, the nucleic acid sequences described in Table 19 lack the indicated 3′ cap (e.g., a 3′ inverted dT cap (3T)). Lower case letters “m”, and “d” denote 2-O-methyl, and deoxy modifications respectively, “s” denotes an internucleotide phopshorothioate substitution, “I” denotes an inosine substitution, and PEG20K indicates a 20 kDa PEG group.

TABLE 19

Sequences and Approximate Binding affinities
of ARC16666 derivatives

| | | | |
|---|---|---|---|
| SEQ | | | |
| ID | | | |
| NO | ARC # | Sequence | K_{D} (nM) |

| | | | |
|---|---|---|---|
| 216 | ARC1666 | mAmGmCmCmUdGmGdG-s-dGmAmCmCmCm | 0.035 |
| | | AmU-s-dI-s-mGdI-s-dGdI-s-dGmCm | |
| | | U-3T | |
| 299 | ARC2340 | mAmGmCmCmUdGmGdG-s-dGmAmCmCmCm | 0.08 |
| | | AmU-s-dI-s-mGdI-s-dGdI-s-dG-s- | |
| | | mCmU-3T | |
| 300 | ARC2341 | mAmGmCmCmUdGmGdG-s-dGmAmCmCmCm | 0.05 |
| | | AmU-s-dI-s-mGdI-s-dGdI-s-dG-s- | |
| | | mC-s-mU-3T | |
| 301 | ARC2342 | mAmGmCmCmUdGmGdG-s-dGmAmCmCmCm | 0.19, |
| | | AmU-s-dI-s-mGdI-s-dGdI-s-dG-s- | .04* |
| | | mC-s-mU-s--3T | |
| 302 | ARC2369 | mAmGmCmCmUdGmGdG-s-dGmAmCmCmCm | .03 |
| | | AmU-s-dI-s-mGdI-s-dGdI-s-dGmCm | |
| | | U-nh-PEG20K | |
| 303 | ARG3084 | mAmGmCmCmUdGmGdG-s-dGmAmCmCmCm | N.D. |
| | | AmU-s-dI-s-mGdI-s-dGdI-s-dGmCm | |
| | | UNH2 | |
| 304 | ARG2969 | mA-s-mGmCmCmUdGmGdG-s-dGmAmCmC | .09 |
| | | mCmAmU-s-dI-s-mGdI-s-dGdI-s-dG | |
| | | mCmU-3T | |
| 305 | ARC2970 | mAmG-s-mCmCmUdGmGdG-s-dGmAmCmC | .04 |
| | | mCmAmU-s-dI-s-mGdI-s-dGdI-s-dG | |
| | | mCmU-3T | |
| 306 | ARC2971 | mAmGmC-s-mCmUdGmGdG-s-dGmAmCmC | .06 |
| | | mCmAmU-s-dI-s-mGdI-s-dGdI-s-dG | |
| | | mCmU-3T | |
| 307 | ARC2972 | mAmGmCmC-s-mUdGmGdG-s-dGmAmCmC | .05 |
| | | mCmAmU-s-dI-s-mGdI-s-dGdI-s-dG | |
| | | mCmU-3T | |
| 308 | ARC2973 | mAmGmCmCmU-s-dGmGdG-s-dGmAmCmC | .05 |
| | | mCmAmU-s-dI-s-mGdI-s-dGdI-s-dG | |
| | | mCmU-3T | |
| 309 | ARC2974 | mAmGmCmCmUdG-s-mGdG-s-dGmAmCmC | .07 |
| | | mCmAmU-s-dI-s-mGdI-s-dGdI-s-dG | |
| | | mCmU-3T | |
| 310 | ARC2975 | mAmGmCmCmUdGmG-s-dG-s-dGmAmCmC | .04 |
| | | mcmAmU-s-dI-s-mGdI-s-dGdI-s-dG | |
| | | mCmU-3T | |
| 311 | ARC2976 | mAmGmCmCmUdGmGdG-s-dG-s-mAmCmC | 0.3 |
| | | mCmAmU-s-dI-s-mGdI-s-dGdI-s-dG | |
| | | mCmU-3T | |
| 312 | ARC2977 | mAmGmCmCmUdGmGdG-s-dGmA-s-mCmC | .05 |
| | | mCmAmU-s-dI-s-mGdI-s-dGdI-s-dG | |
| | | mCmU-3T | |
| 313 | ARC2978 | mAmGmCmCmUdGmGdG-s-dGmAmC-s-mC | .05 |
| | | mCmAmU-s-dI-s-mGdI-s-dGdI-s-dG | |
| | | mCmU-3T | |
| 314 | ARC2979 | mAmGmCmCmUdGmGdG-s-dGmAmCmC-s- | .05 |
| | | mCmAmU-s-dI-s-mGdI-s-dGdI-s-dG | |
| | | mCmU-3T | |
| 315 | ARC2980 | mAmGmCmCmUdGmGdG-s-dGmAmCmCmC- | .04 |
| | | s-mAmU-s-dI-s-mGdI-s-dGdI-s-dG | |
| | | mCmU-3T | |
| 316 | ARC2981 | mAmGmCmCmUdGmGdG-s-dGmAmCmCmCm | .02 |
| | | A-s-mU-s-dI-s-mGdI-s-dGdI-s-dG | |
| | | mCmU-3T | |
| 317 | ARC2982 | mAmGmCmCmUdGmGdG-s-dGmAmCmCmCm | .03 |
| | | AmU-s-dI-s-mG-s-dI-s-dGdT-s-dG | |
| | | mCmU-3T | |
| 318 | ARC2983 | mAmGmCmCmUdGmGdG-s-dGmAmCmCmCm | .02 |
| | | AmU-s-dI-s-mGdI-s-dG-s-dI-s-dG | |
| | | mCmU-3T | |
| 319 | ARC3262 | mAmGmCmCmUdGmGmG-s-dGmAmCmCmCm | 0.16 |
| | | AmU-s-dI-s-mGdI-s-dGdI-s-dGmCm | |
| | | U-3T | |
| 320 | ARC3263 | mAmGmCmCmUdGmGdG-s-mGmAmCmCmCm | .026 |
| | | AmU-s-dI-s-mGdI-s-dGdI-s-dGmCm | |
| | | U-3T | |
| 321 | ARC3264 | mAmGmCmCmUdGmGdG-s-dGmAmCmCmCm | 0.36 |
| | | AmU-s-mI-s-mGdI-s-dGdI-s-dGmCm | |
| | | U-3T | |
| 322 | ARC3265 | mAmGmCmCmUdGmGmG-s-mGmAmCmCmCm | 0.29 |
| | | AmU-s-dI-s-mGdI-s-dGdI-s-dGmcm | |
| | | U-3T | |
| 323 | ARC3266 | mAmGmCmCmUdGmGmG-s-dGmAmCmCmCm | 1.3 |
| | | AmU-s-mI-s-mGdI-s-dGdI-s-dGmCm | |
| | | U-3T | |
| 324 | ARC3267 | mAmGmCmCmUdGmGdG-s-mGmAmCmCmCm | 0.2 |
| | | AmU-s-mI-s-mGdI-s-dGdI-s-dGmCm | |
| | | U-3T | |
| 325 | ARC3268 | mAmGmCmCmUdGmGmG-s-mGmAmCmCmCm | 0.6 |
| | | AmU-s-mI-s-mGdI-s-dGdI-s-dGmCm | |
| | | U-3T | |
| 326 | ARC3269 | mAmGmCmCmUdGmGmG-s-dGmAmCmCmCm | 0.52 |
| | | AmU-s-dI-s-mGdI-s-dGdI-s-dG-s- | |
| | | mC-s-mU-s--3T | |
| 327 | ARC3270 | mAmGmCmCmUdGmGdG-s-mGmAmCmCmCm | 0.04 |
| | | AmU-s-dI-s-mGdI-s-dGdI-s-dG-s- | |
| | | mC-s-mU-s--3T | |
| 328 | ARC3271 | mAmGmCmCmUdGmGdG-s-dGmAmCmCmCm | 0.23 |
| | | AmU-s-mI-s-mGdI-s-dGdI-s-dG-s- | |
| | | mC-s-mU-s--3T | |
| 329 | ARC3272 | mAmGmCmCmUdGmGmG-s-mGmAmCmCmCm | 0.12 |
| | | AmU-s-dI-s-mGdI-s-dGdI-s-dG-s- | |
| | | mC-s-mU-s--3T | |
| 330 | ARC3273 | mAmGmCmCmUdGmGmG-s-dGmAmCmCmCm | 0.86 |
| | | AmU-s-mI-s-mGdI-s-dGdI-s-dG-s- | |
| | | mC-s-mU-s--3T | |
| 331 | ARC3274 | mAmGmCmCmUdGmGdG-s-mGmAmCmCmCm | 0.12 |
| | | AmU-s-mI-s-mGdI-s-dGdI-s-dG-s- | |
| | | mC-s-mU-s--3T | |
| 332 | ARC3275 | mAmGmCmCmUdGmGmG-s-mGmAmCmCmCm | 0.67 |
| | | AmU-s-mI-s-mGdI-s-dGdI-s-dG-s- | |
| | | mC-s-mU-s--3T | |
| 333 | ARC3276 | mAmGmUmAmGmCmCmUdGmGdG-s-dGmAm | 0.03 |
| | | CmCmCmAmU-s-dI-s-mGdI-s-dGdI- | |
| | | s-dGmCmUmAmCmU-3T | |

*indicates two independent measurements
N.D. indicates not determined

### Example 2H

### Synthesis of Aptamer-5′-PEG Conjugates

The oligonucleotide 5′ NH₂ mA mG mC mC mU dG mG dG-s-dG mA mC mC mC mA mU-s-dI-s-mG dI-s-dG dI-s-dG mC mU-idT 3′ (ARC1666, SEQ ID NO 216) was synthesized on an AKTA OligoPilot 100 synthesizer (GE Healthcare, Uppsala, Sweden) according to the recommended manufacturer's procedures using standard commercially available 2′-OMe RNA, deoxyinosine and DNA phosphoramidites (Glen Research, Sterling, Va.) and a inverted deoxythymidine CPG support. A terminal amine functions was attached with a 5′-amino-modifier C6-TFA (Glen Research, Sterling, Va.). After deprotection, the oligonucleotide was purified by ion exchange chromatography on Super Q 5PW (30) resin (Tosoh Biosciences) and ethanol precipitated.

Aliquots of the 5′-amine-modified aptamer were conjugated to different PEG moieties post-synthetically (e.g., 40 kDa, 60 kDa PEG moieties). Aptamers were dissolved in a water/DMSO (1:1) solution to a concentration between 1.5 and 3 mM. Sodium carbonate buffer, pH 8.5, was added to a final concentration of 100 mM, and the oligo was reacted overnight with a 1.7-3 fold molar excess of the desired PEG reagent (e.g. 40 kDa Sunbright GL2-400NP p-nitrophenyl carbonate ester [NOF Corp, Japan], 40 kDa or 60 kDa mPEG2-NHS ester [Nektar, Huntsville Ala.]) dissolved in an equal volume of acetonitrile. The resulting 40 kDa or 60 kDa PEGylated products were purified by ion exchange chromatography on Super Q 5PW (30) resin (Tosoh Biosciences), and desalted using reverse phase chromatography performed on Amberchrom CG300-S resin (Rohm and Haas), and lyophilized.

The resulting PEGylated aptamer sequences are listed in Table 20 below. Lower case letters “f”, “m”, and “d” denote 2′-fluoro, 2-O-methyl, and deoxy modifications respectively, “s” denotes an internucleotide phopshorothioate substitution, “I” denotes an inosine substitution for guanosine, “NH” denotes an amine to facilitate chemical coupling, and “3T” denotes a 3′ inverted dT.

TABLE 20

5′ PEG conjugates of anti-IgE aptamer
ARC1666

ARC1787 (SEQ ID NO 293)
40K-NH-mAmGmCmCmUdGmGdG-s-dGmAmCmCmCmAmU-s-dI-s-mG
dI-s-dGdI-s-dGmCmU-3T
ARC1788 (SEQ ID NO 294)
60KNH--mAmGmCmCmUdGmGdG-s-dGmAmCmCmCmAmU-s-dI-s-mG
dI-s-dGdI-s-dGmCmU-3T

PEGylation had little to no effect on aptamer function, measured by a cell based assay described in Example 3 below.

### Example 2I

### Synthesis of Aptamer-3′-5′-PEG Conjugates

The oligonucleotide 5′ NH₂ mA mG mC mC mU dG mG dG-s-dG mA mC mC mC mA mU-s-dI-s-mG dI-s-dG dI-s-dg mC mU —NH₂ 3′ (ARC1784, SEQ ID NO 297) was synthesized on an AKTA OligoPilot 100 synthesizer (GE Healthcare Uppsala, Sweden) according to the recommended manufacturer's procedures using standard commercially available 2′-OMe RNA, deoxyinosine and DNA phosphoramidites (Glen Research, Sterling, Va.) and a 3′-phthalimide-amino-modifier C6 CPG support (Glen Research, Sterling, Va.). A terminal amine functions was attached with a 5′-amino-modifier C6-TFA (Glen Research, Sterling, Va.). After deprotection, the oligonucleotides was purified by ion exchange chromatography on Super Q 5PW (30) resin (Tosoh Biosciences) and ethanol precipitated.

Aliquots of the 3′-5′-diamine-modified aptamer were conjugated to different PEG moieties post-synthetically (e.g., 20 kDa and 30 kDa PEG moieties). Aptamers were dissolved in a water/DMSO (1:1) solution to a concentration between 1.5 and 3 mM. Sodium carbonate buffer, pH 8.5, was added to a final concentration of 100 mM, and the oligo was reacted overnight with a 2.7-3.5 fold molar excess of the desired PEG reagent (e.g., 20 kDa or 30 kDa Sunbright GL2-400NP p-nitrophenyl carbonate ester [NOF Corp, Japan]) dissolved in an equal volume of acetonitrile. The resulting 2×20 kDa or 2×30 kDa PEGylated products were purified by ion exchange chromatography on Super Q 5PW (30) resin (Tosoh Biosciences), and desalted using reverse phase chromatography performed on Amberchrom CG300-S resin (Rohm and Haas), and lyophilized.

The resulting bi-PEGylated aptamer sequences are listed in Table 21 below. Lower case letters “f”, “m”, and “d” denote 2′-fluoro, 2-O-methyl, and deoxy modifications respectively, “s” denotes an internucleotide phopshorothioate substitution, “I” denotes an inosine substitution for guanosine, and “NH” denotes an amine to facilitate chemical coupling.

TABLE 21

3′-5′10 -PEG Conjugates of anti-IgE aptamer
ARC1666

ARC1785 (SEQ ID NO 295)
PEG20K-NH-mAmGmCmCmUdGmGdG-s-dGmAmCmCmCmAmU-s-dI-
s-mGdI-s-dGdI-s-dGmCmU-NH-PEG20K
ARC1790 (SEQ ID NO 296)
PEG30K-NH-mAmGmCmGmUdGmGdG-s-dGmAmCmCmCmAmU-s-dI-
s-mGdI-s-dGdI-s-dGmCmU-NH-PEG30K

3′-5′ PEGylation had little to no effect on aptamer function as measured by a cell based assay described in Example 3 below.

### Example 2J

### ARC1666 3′ Truncants

3′-truncants of ARC1666 (n-1, n-2, and n-3) were synthesized, purified. Dot blot binding assays were performed on ARC2343 (n-1), ARC2344 (n-2) and ARC2345 (n-3) to assess the activity of n-1, -2 and -3 ARC1666. The sequences of ARC2343, ARC2344 and ARC2345 are listed in Table 22 below. For K_{D} determination, aptamers were 5′ end labeled with γ-³²P ATP, combined with h-IgE and incubated at room temperature for 30 minutes in 1×Dulbecco's PBS buffer plus 0.1 mg/mL BSA in a final volume of 30 μL. The mixture was applied to a dot blot apparatus (Schleicher and Schuell Minifold-1 Dot Blot, Acrylic), assembled (from top to bottom) with nitrocellulose, nylon, and gel blot membranes. RNA that is bound to protein is captured on the nitrocellulose filter; whereas the non-protein bound RNA is captured on the nylon filter. Data was fit to the equation (amp1.1/(1+K_{D1}/[IgE])+amp1.2/(1+K_{D2}/[IgE]))+background in which amp1.1 and amp1.2 represent the plateau values for two phases of a biphasic saturation plot and K_{D1} and K_{D2} represent the dissociation constants for each interaction to the resulting data. The resulting calculated K_{D} values are listed in Table 23 below.

For the sequences listed in Table 22, lower case letters “m”, and “d” denote 2-O-methyl, and deoxy modifications respectively, “s” denotes an internucleotide phopshorothioate substitution, “I” denotes an inosine substitution.

TABLE 22
Sequences of ARC1666 3′-Truncants

ARC2343 (SEQ ID NO 334)
mAmGmCmCmUdGmGdG-s-dGmAmCmCmCmAmU-s-dI-s-mGdI-s-dG
dI-s-dGmCmU
ARC2344 (SEQ ID NO 335)
mAmGmCmCmUdGmGdG-s-dGmAmCmCmCmAmU-s-dI-s-mGdI-s-dG
dI-s-dGmC
ARC2345 (SEQ ID NO 336)
mAmGmCmCmUdGmGdG-s-dGmAmCmCmCmAmU-s-dI-s-mGdI-s-dG
dI-s-dG

TABLE 23
K_{D}'s for ARC1666 3′-truncants binding to IgE

| SEQ ID | | |
|---|---|---|
| NO | Aptamer | K_{D} (nM)_{—} |
| 216 | ARC1666 | 0.06 |
| 334 | ARC2343 | 0.09 |
| 335 | ARC2344 | 0.41 |
| 336 | ARC2345 | 29.3 |

### Example 3

### Functional Cell Assays

### Example 3A

### Receptor (FcεR1) Binding Inhibition ELISA

A panel of the rRfY IgE aptamers (described above in Example 1A) were tested for the ability to inhibit complex formation between h-IgE and soluble, FcεR1_{α—}Fc (purified in-house) using an ELISA assay. FcεR1 (100 μL, 10 μg/mL) in 1×PBS was incubated in the wells of a Nunc Maxisorb 96 well plate overnight at 4° C. to coat the surface of the wells. The supernatants were removed, the wells were washed 3 times with 120 μl 1×PBS, and the wells were blocked with 300 mL 1×PBS plus 0.2% Tween-20 at 4° C. for two hours. After blocking, the wells were washed three times with 1×PBS. Various concentrations of aptamer were next incubated with 0.5 nM h-IgE in 100 μL PBS plus 0.05% Tween-20 at room temperature for 30 minutes, and then the mixtures were added to the assay well and incubated at room temperature for 1 hour. The wells were then washed 5 times with 120 μL 1×PBS. Bound h-IgE was detected by the addition of HRP-labeled anti-h-IgE polyclonal antibody (Goat anti-human IgE-HRP (074-1004) (KPL, Gaithersburg, Md.)). Quantablue substrate (Pierce, Rockford, Ill.) was used to detect peroxidase activity. 100 μl of Quantablue substrate was added to each well and incubated at room temperature for 15 minutes. Next, 100 μl of the provided stop solution was added to each well, and the plates were read on a SpectraMax 96 well plate reader at excitation/emission of 325 nm and 420 nm respectively. The relative fluorescence units (RFU) of each well were used to calculate IC50's. Table 24 below shows the IC₅₀s calculated for FcεRI_{α} receptor inhibition with various aptamers from the rRfY selection.

TABLE 24
Receptor (FcεR1) binding inhibition - rRfY aptamers

| SEQ ID | |
|---|---|
| NO | IC₅₀ (nM) |
| 11 | 2.6 |
| 12 | 5.0 |
| 13 | 4.3 |
| 14 | 26.8 |
| 18 | 51.3 |
| 21 | 3.7 |
| 19 | 7.4 |

Functional Activity of ARC1666 and ARC1666 derivatives in Cynomolgus Monkey Serum

ARC1666 (SEQ ID NO 216), a highly potent variant of ARC445 (SEQ ID NO 101) was identified during phase 5.3 of the ARC445 aptamer medicinal chemistry efforts (see Example 2E) in which derivative molecules of ARC445 (SEQ ID NO 101) designed to contain multiple dG to dI substitutions were synthesized, purified, and assayed for binding activity. The functional activity of ARC1666 in cynomolgus monkey sera was measured using the FcεR1α binding inhibition ELISA described above.

To measure the functional activity of ARC1666 in cynomolgus monkey sera, 1 μM of ARC1666 was incubated in 1 mL 99% cynomolgus monkey serum at 37° C. Aliquots were removed at various time points (0, 1, 2, 4, 8, 12, 24, and 48 hours), quenched with 20 μL 500 mM EDTA and flash frozen. Cynomolgus monkey sera containing ARC1666 was tested for the ability to inhibit complex formation between h-IgE (Athens Research, Athens, Ga.) and soluble FcεR1_{α—}Fc (purified in-house), using an ELISA assay as follows. FcεR1 (100 μL, 0.5 μg/mL) in 1×PBS was incubated in the wells of a Nunc Maxisorb 96 well plate overnight at 4° C. to coat the surface of the wells. The supernatants were removed, the wells were washed 3 times with 200 μl 1×TBS+0.05% Tween-20, and the wells were blocked with 200 μL 5% milk in 1×TBS plus 0.05% Tween-20 at room temperature for one hour. After blocking, the wells were washed three times with 1×TBS+0.05% Tween-20.

A dilution of ARC1666 in cynomolgus monkey sera from each time point (typically a 1:15 dilution) was next incubated with 2.5 nM h-IgE in 100 μL TBS plus 0.05% Tween-20 at room temperature for 15 minutes, and then the mixtures were added to the assay wells and incubated at room temperature for 1 hour. The wells were then washed 3 times with 200 μL 1×TBS+0.05% Tween-20. Bound h-IgE was detected by the addition of HRP-labeled anti-h-IgE polyclonal antibody (Goat anti-human IgE-HRP (074-1004) (KPL, Gaithersburg, Md.)). After forty-five minute incubation with anti-h-IgE polyclonal antibody, wells were washed 3 times with 200 μL 1×TBS+0.05% Tween-20. Quantablue substrate (Pierce, Rockford, Ill.) was used to detect peroxidase activity. 100 μl of Quantablue substrate was added to each well and incubated at room temperature for 1 minute. Next, 100 μl of the provided stop solution was added to each well, and the plates were read on a VersaMax 96 well plate reader at 450 nm.

Aptamer concentration in each sample was determined from a standard curve based on percent inhibition in the ELISA for each aptamer as follows. For the standard curve, ARC1666 was serially diluted (1:1.5) in a percentage of cynomolgus monkey sera (0, 3.9 nM-45 nM) and run in the receptor (FcεR1) binding inhibition ELISA described above. The final percentage of cynomolgus monkey sera used to generate the standard curve depended on the dilution factor of the samples (typically 1:15, described above) to normalize for percentage of monkey sera used in both the standard curve and samples. Absorbance values at 450 nm (“ABS”) versus log aptamer concentration was graphed and a linear curve fit (y=mx+b) was completed where “y” is the measured absorbance value, at 450 nm, “m” is the slope (provided by the curve fit), “b” is the y-intercept (provided by the curve fit), and “x” is the amount of functional aptamer, to be calculated. The following equation was used to calculate the final amount of active aptamer at the various time points: Amount of functional aptamer [nM]=[[10̂((ABS-b)/(m))]*dilution factor]. Based on this study, ARC1666 retained at least 58% functional activity at the 2 hour time point in the ELISA assay.

The functional activity of ARC1666 derivatives (described in Example 2G) in cynomolgus monkey serum was also measured using the FcεR1α binding inhibition ELISA. Calculation of aptamer concentration in each sample was determined from a standard curve based on percent inhibition in the ELISA for each aptamer, as previously described. The results for ARC2342 revealed that the introduction of multiple phosphorothioate substitutions at the 3′-end of ARC1666 (Phase I of the ARC1666 aptamer medicinal chemistry process), did not have an adverse affect on the functional activity of ARC2342. The functional activity of ARC2342 in cynomolgus monkey serum was better than that of ARC1666, showing at least 71% activity at the 2 hour time point. Additionally, the results for ARC2982, ARC2983, ARC2979, ARC2970, ARC2971, ARC2972, ARC2976, and ARC2977 revealed that the introduction of additional phosphorothioate linkages at sites which did not contain phosphorothioate linkages in the context of ARC1666 (Phase 2 of the ARC1666 aptamer medicinal chemistry process) did not have an These constructs showed a range of 50% to 72% activity in cynomolgus monkey serum at the 2 hour time point.

### Example 3B

### Biacore Analysis

To verify the functional activity of ARC1666 in cynomolgus monkey serum (described in Example 3A), Biacore analysis was performed. The binding of target protein (IgE) was used to measure the functional concentration of ARC1666 incubated with Cynomolgus monkey serum.

All biosensor binding measurements were performed at 25° C. using a BIACORE 2000 equipped with a research-grade CM5 biosensor chip (BIACORE Inc. Piscataway, N.J.). Purified recombinant human IgE (Athens research biotechnology, Athens, Ga.) was immobilized to the biosensor surface using amino-coupling chemistry. To achieve this, the surfaces of two flow cells were first activated for 7 minutes with a 1:1 mixture of 0.1 M NHS (Nhydroxysuccinimide) and 0.4 M EDC (3-(N,Ndimethylamine) propyl-N-ethylcarbodiimide) at a flow rate of 5 μl/min. After surface activation, one flow cell was injected with 50 μg/ml of IgE at a rate of 10 μl/min for 20 minutes to allow for establishment of covalent bonds to the activated surface. Next, 1 M ethanolamine hydrochloride pH 8.5 was injected for 7 minutes at a rate of 5 μl/min to inactivate residual esters. As a negative control, a blank flow cell was prepared by injecting 1 M ethanolamine hydrochloride pH 8.5 for 7 minutes to inactivate residual esters without protein injection.

The same ARC1666 samples generated for the ELISA-based analysis, as described above in Example 3A, were assayed using Biacore. To measure the active concentration of ARC1666 in Cynomolgus monkey serum, all samples were diluted 25 fold in HBS-P for a final Cynomolgus monkey serum concentration of 4% before injecting into the Biacore 2000. To establish a standard curve, ARC1666 was serially diluted (100 nM to 3.1 nM) into HBS-P buffer (10 mM HEPES pH7.4, 150 mM NaCl, 0.005% Surfactant 20) supplemented with 4% Cynomolgus monkey serum and 50 mM EDTA. All diluted samples were injected into the Biacore 2000 one at a time for binding at a rate of 20 μl/min continuously for 5 min followed by a period of no injection for a duration of 3 minutes. To test subsequent concentrations, the surface was regenerated by injecting 1N NaCl for 60 seconds at a rate of 20 μl/min.

RU peak response at the end of each binding phase was plotted against the various concentrations of ARC1666 to generate a standard curve. The RU response unit was linear over the concentration range of 3.1 nM to 50 nM in HBS-P with 4% Cynomolgus monkey serum. To calculate the functional ARC1666 concentration at each serum incubation period, RU response unit was converted to concentration using a linear response standard curve multiplied by the dilution factor of 25. The functional activity of ARC1666 measured using Biacore correlated well with the functional activity as measured using the FcεR1α-Fc binding inhibition ELISA.

### Example 3C

### Receptor (FcεR1) Binding Inhibition by FACS

A panel of aptamers representing rRfY, dRmY and DNA compositions was tested for the ability to inhibit h-IgE binding to FcεR1 expressed on the surface of rat basophilic leukemia (RBL) cells. SX38 cells, an RBL cell line (Harvard University, Cambridge, Mass.), stably expressing the human α, β, and γ chains of the h-IgE receptor, were used in a flow cytometry h-IgE binding assay. The cells were cultured in Eagle's Minimal Essential Medium (MEM) with 16% fetal bovine serum (FBS) and 1 mg/mL G418 (Invitrogen) at 37° C., 5% CO2. One million SX38 cells in 1×DPBS with 1% BSA (Sigma, St. Louis, Mo.) and 0.03% NaAzide (FACs buffer) (VWR, West Chester, Pa.) were aliquotted into an appropriate number of wells in a 96 well plate and incubated on ice for 30 minutes. For each aptamer concentration, a 2× mixture of aptamer plus h-IgE (Athens Research and Technology, Athens, Ga.) was incubated on ice for 30 minutes. The aptamer and h-IgE mixture was added to the SX38 cells to obtain a final 1× concentration and incubated on ice for an additional 30 minutes. Final aptamer concentrations ranging from 0-1 μM were screened against 3 μg/mL h-IgE (15 nM). The cells were then washed 3× with FACs buffer to remove any h-IgE that did not bind to the receptor. To detect h-IgE binding, an anti-h-IgE-FITC antibody (QED Biosciences, San Diego, Calif.) was added to the cells and incubated for 30 minutes on ice. After the incubation, the cells were washed 3× with FACs buffer to remove any unbound antibody. The cells were resuspended in FACs buffer and were analyzed using FACSCAN (BD Biosciences, San Jose, Calif.). h-IgE alone and h-IgE versus the naïve pool were included as positive and negative controls, respectively. h-IgE aptamer activity was measured by percent inhibition of h-IgE binding to the cells. Mean fluorescence intensity values were used to calculate percent inhibition. Table 25 shows the IC₅₀ for various rRfY, dRmY, and DNA clones selected as calculated by FACS assay.

TABLE 25
rRfY, dRmY, DNA clones: Receptor (FceR1)
binding inhibition by FACS assay

| SEQ ID | | | |
|---|---|---|---|
| NO | ARC# | Composition | IC₅₀ (nM) |
| 12 | | rRfY | 7.1 |
| 11 | | rRfY | 14 |
| 21 | | rRfY | 14.4 |
| 18 | | rRfY | 35 |
| 13 | | rRfY | 10.3 |
| 19 | | rRfY | 9.9 |
| 14 | | rRfY | 17.3 |
| 91 | | rRfY | 3.8 |
| 93 | | rRfY | 12.5 |
| 98 | ARC442 | dRmY | 58.9 |
| 99 | ARC443 | dRmY | 30.2 |
| 100 | ARC444 | dRmY | 52.4 |
| 102 | ARC445 | dRmY | 22.6 |
| 102 | ARC446 | dRmY | 33.8 |
| 149 | | DNA | 55.9 |
| 149 (with 3′ idT) | | DNA | 35.8 |
| 152 | | DNA | 17.4 |
| 151 | | DNA | 17.1 |
| 151 (with 3′ idT) | | DNA | 10.6 |
| 155 | | DNA | 14.4 |
| 156 | | DNA | 9.7 |

*idT: same sequence with inverted dT residue at the 3′-end
N.D.: not determined

Several of the modified derivatives of ARC445 (SEQ ID NO 101) (described in Example 2E) as well as ARC656 (SEQ ID NO 157) (described in Example 2F) were also tested for h-IgE receptor (FceR1) binding inhibition using the FACS assay described above with the following modifications: for each aptamer concentration, a 2× mixture of aptamer plus h-IgE (Athens Research and Technology, Athens, Ga.) was incubated on ice for 30 minutes; the aptamer and h-IgE mixture was added to the SX38 cells to obtain a final 1× concentration and incubated on ice for an additional 2 hours; and final aptamer concentrations ranging from 0-1 μM were screened against 0.5 μg/mL h-IgE (2.5 nM. The calculated IC₅₀ values are summarized in Table 26.

TABLE 26
ARC445 modified Derivatives; ARC656: Receptor
(FceR1) binding inhibition by FACS assay

| SEQ ID | | FACs IC₅₀ |
|---|---|---|
| NO | ARC # | (nM) |
| 101 | ARC445 | 10.8 |
| 176 | ARC1335 | 7.3 |
| 178 | ARC1337 | 4.7 |
| 179 | ARC1382 | 6.9 |
| 180 | ARC1383 | 7.5 |
| 181 | ARC1384 | 4.4 |
| 182 | ARC1572 | 22.8 |
| 183 | ARC1573 | 69.4 |
| 212 | ARC1641 | 1.8 |
| 213 | ARC1642 | 1.8 |
| 214 | ARC1643 | 8.0 |
| 215 | ARC1644 | 5.2 |
| 216 | ARC1666 | 1.7 |
| 217 | ARC1667 | 2.3 |
| 218 | ARC1728 | 13.2 |
| 219 | ARC1729 | 2.9 |
| 157 | ARC656 | 21.2 |

The PEG-conjugated aptamers described in Examples 2H and 2I were all active in the FACS assay, under the conditions described directly above. The calculated IC₅₀ values are summarized in Table 27 below. As can be seen from the data in Table 27, various types of PEG conjugation had little effect on aptamer function.

TABLE 27

| SEQ ID | | | FACS IC₅₀ |
|---|---|---|---|
| NO | PEG | ARC # | (nM) |
| 295 | 2 × 20 kDa | ARC1785 | 2.0 |
| | (linear) | | |
| 296 | 2 × 30 kDa | ARC1790 | 0.89 |
| | (linear) | | |
| 293 | 40 kDa | ARC1787 | 1.2 |
| | branched | | |
| 294 | 60 kDa | ARC1788 | 1.2 |
| | branched | | |

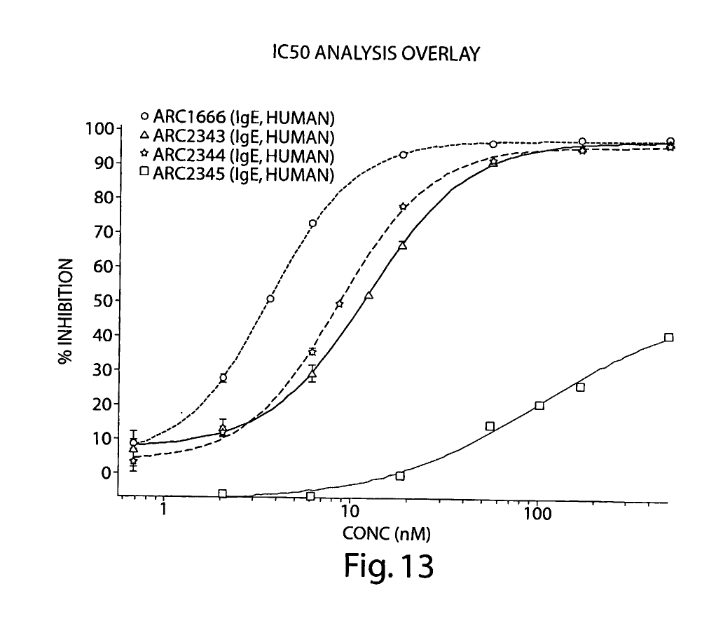

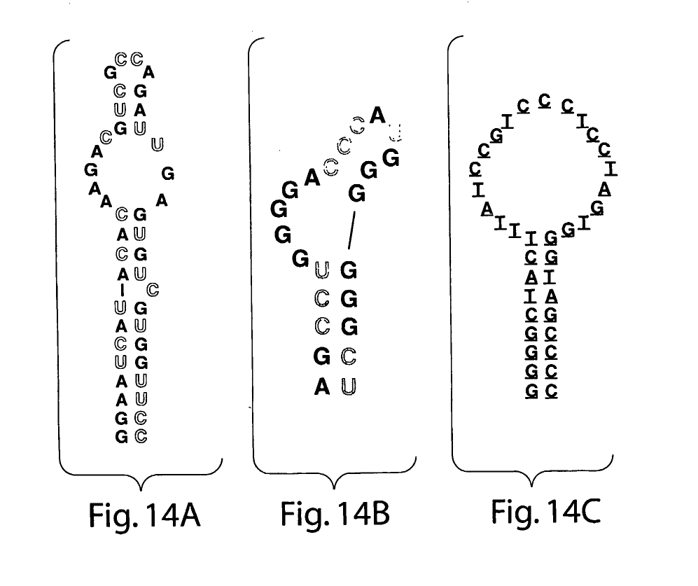

The ARC1666 3′ truncants described in Example 2J were tested in the FACS assay under the conditions described directly above. As can be seen from FIG. 13, ARC2343 and ARC2344, (n-1 and n-2 respectively) both retained within 10-fold functional activity as compared to the full length ARC1666 while the functional activity of ARC2345 was present but significantly reduced compared to ARC1666. The functional activity corresponds to the binding activity described above in Example 2J.

### Example 3D

### Aptamer Cross Reactivity with Cynomolgous Monkey IgE

Aptamers were tested for the ability to inhibit complex formation between cynomolgous monkey IgE and FcεR1_{α}-Fc using an ELISA assay. FcεR1_{α}-Fc (100 mL, 5 μg/mL) in PBS was incubated in the wells of a Nunc Maxisorb 96 well plate overnight at 4° C. to coat the surface of the wells. The supernatants were removed, the wells were washed 3 times with 120 μl 1×PBS, and the wells were blocked with 300 μL PBS plus 0.2% Tween-20 at 4° C. for 2 hours. The wells were washed 3 times with 120 μL 1×PBS. Various concentrations of aptamer were next incubated with 0.5 ng h-IgE in 100 μL PBS (or cynomolgous monkey serum (Sigma) diluted to 100% with same) at room temperature for 30 minutes, and then the mixtures were added to the assay well and incubated at room temperature for 1 hour. The wells were then washed 5 times with 120 μL PBS. Bound IgE (both monkey and human) was detected by the addition of HRP-labeled anti-IgE polyclonal antibody (Goat anti-human IgE-HRP (074-1004) (KPL, Gaithersburg, Md.)). Quantablue substrate (Pierce, Rockford, Ill.) was used to detect peroxidase activity. 100 μl of Quantablue substrate was added to each well and incubated at room temperature for 15 minutes. Next, 100 μl of the provided stop solution was added to each well, and the plates were read on a SpectraMax 96 well plate reader at excitation/emission of 325 nm and 420 nm respectively. The relative fluorescence units (RFU) of each well were used to calculate IC₅₀'s).

The presence of the following DNA aptamers with nucleic acid sequences according to SEQ ID NO 149 (with a 3′inverted deoxy thymidine) and SEQ ID NO 151 (with a 3′ inverted deoxy thymidine), or the rRfY aptamers with nucleic acid sequences according to SEQ ID NO 90 and SEQ ID NO 93, at concentrations up to 250 nM and 1 μM respectively, did not inhibit monkey binding to FcεR1-Fc, while the dRmY aptamers ARC445 (SEQ ID NO 101) and ARC1666 (SEQ ID NO 216) were quite potent at blocking the interaction (ARC445, IC₅₀monkey=161 nM; IC₅₀human=63 nM; ARC1666, IC₅₀monkey=8 nM; IC₅₀human=5 nM), indicating this molecule is cross reactive with cynomolgous monkey IgE and human IgE. Additional control experiments showed that ARC445 did not inhibit detection of monkey IgE in this assay format.

FIG. 14 shows possible secondary structures for rRfY, dRmY and DNA minimized aptamers showing highest potency in IgE:FcεR1 binding inhibition by FACS. Left: SEQ ID NO 91 (rRfY), outlined residues are 2′-F; middle: ARC445 (SEQ ID NO 101) (dRmY), underlined residues are 2′-deoxy, outlined residues are 2′-OMe; right ARC475 (SEQ ID NO 151) (DNA), underlined residues are 2′-deoxy.

### Example 3E

### Inhibition of Histamine Release

IgE aptamer cell based activity was measured using a histamine release assay using SX38 cells (Dana Farber Cancer Institute, Boston, Mass.), an RBL cell line stably expressing the human α, β, and γ chains of the h-IgE receptor. Because supernatants of SX38 cells in the presence of h-IgE and an anti-h-IgE cross linking antibody contain more histamine than aptamer blocked cells, a competitive histamine ELISA (Immuno-Biological Laboratories, Minneapolis, Minn.) can be used to quantify the levels of histamine released into the supernatants of SX38 cells treated with h-IgE and h-IgE cross linking antibody+/−IgE aptamer.

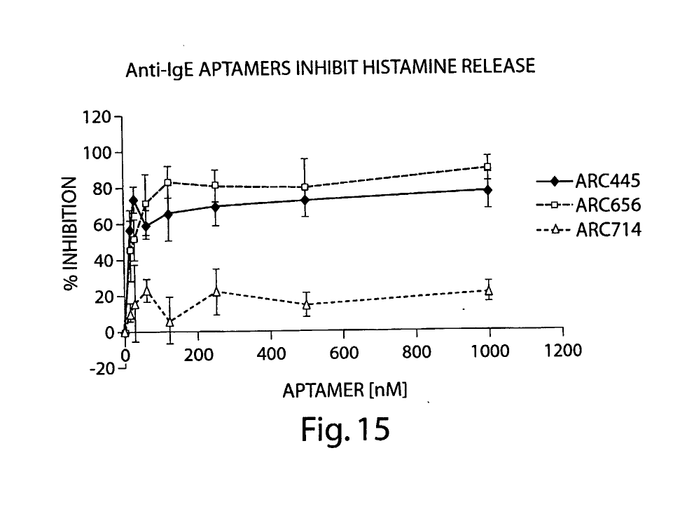

The assay was performed as follows. Two hundred thousand (200,000) SX38 cells per well were plated in MEM plus 16% FBS in a 24 well plate one day prior to the experiment. The next day, appropriate concentrations of aptamer (8 point titration of 2-fold serial dilutions beginning with 1 μM) plus 3 ug/mL of h-IgE (Athens Research and Technology, Athens, Ga.) were incubated together for 30 minutes in MEM plus 16% FBS. The mixture was then added to the SX38 cells and incubated at 37° C., 5% CO₂ for 30 minutes. Each concentration was tested in quadruplicate. The cells were washed three times with MEM plus 16% FBS, then incubated for an additional 5.5 hours in MEM plus 16% FBS. An h-IgE cross-linking antibody (QED Biosciences, San Diego, Calif.) was added to the media on the cells at a concentration of 1 ug/mL and incubated for an additional 2 hours. The supernatants were collected and frozen at −20° C. until use in the histamine ELISA. The histamine ELISA was used according to manufacturer's recommendations. h-IgE alone and h-IgE versus the naïve pool were included as positive and negative controls, respectively. IgE aptamer activity was measured by percent inhibition of histamine release into the supernatants as shown in FIG. 15 (wherein ARC445 is SEQ ID NO 101, ARC656 is SEQ ID NO 157 and ARC714 is a non-binding negative control).

Several modified derivatives of ARC445 (SEQ ID NO 101) (described in Example 2E), along with the DNA aptamer, ARC656 (SEQ ID NO 157) (described in Example 2F) were also tested for their ability to inhibit histamine release in SX38 cells as described above. The calculated IC₅₀s for the ARC445 derivatives tested are summarized in Table 28.

TABLE 28
ARC445 Modified Derivatives; ARC656: Inhibition
of Histamine Release in SX38 cells

| SEQ ID | | Histamine Release IC₅₀ |
|---|---|---|
| NO | ARC # | (nM) |
| 101 | ARC445 | 31.28 |
| 176 | ARC1335 | 33.69 |
| 181 | ARC1384 | 59.0 |
| 216 | ARC1666 | 19.4 |
| 157 | ARC656 | 16.9 |

### Example 4

### PK Studies

In Examples 4, all mass based concentration data in this example refers only to the molecular weight of the oligonucleotide portion of the aptamer, irrespective of the mass conferred by PEG conjugation.

### Example 4A

### Plasma Stability of Anti-IgE Aptamers

A subset of the aptamers identified in the aptamer medicinal chemistry process was assayed for nuclease stability in both human and rat plasma. Plasma nuclease degradation was measured on denaturing polyacrylamide gel electrophoresis as described below. Briefly, for plasma stability determination, chemically synthesized aptamers were purified using denaturing polyacrylamide gel electrophoresis, 5′ end labeled with γ-³²P ATP and then gel purified again. Trace ³²-P labeled aptamer was incubated in the presence of 100 nM unlabeled aptamer in 95% human plasma (or 95% rat plasma) in a 200 microliter binding reaction. The reaction for the time zero point was made separately with the same components except that the plasma was replaced with PBS. This insured that the amount or radioactivity loaded on gels was consistent across an experiment. Reactions were incubated at 37° C. in a thermocycler for the 1, 3, 10, 30 and 100 hours unless otherwise specified. At each time point, 20 microliters of the reaction was removed, combined with 200 microliters of formamide loading dye and flash frozen in liquid nitrogen and stored at −20° C. After the last time point was taken, frozen samples were thawed and 20 microliters was removed from each time point. SDS was then added to the small samples to a final concentration of 0.1%. The samples were then incubated at 90° C. for 10-15 minutes and loaded directly onto a 15% denaturing PAGE gel and run at 12 W for 35 minutes. Radioactivity on the gels was quantified using a Storm 860 phosphoroimager system (Amersham Biosciences, Piscataway, N.J.). The percentage of full length aptamer at each time point was determined by quantifying the full length aptamer band and dividing by the total counts in the lane. The fraction of full length aptamer at each time-point was then normalized to the percentage full length aptamer of the 0 hour time-point. The fraction of full length aptamer as a function of time was fit to the equation:

m1*ê(−m2*m0)

where m1 is the maximum % full length aptamer (m1=100); and m2 is the rate of degradation.

The half-life of the aptamer (T_{1/2}) is equal to the (ln 2)/m2.

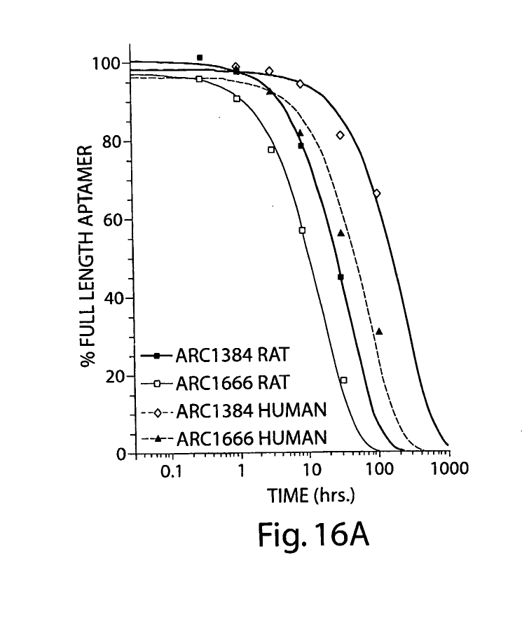

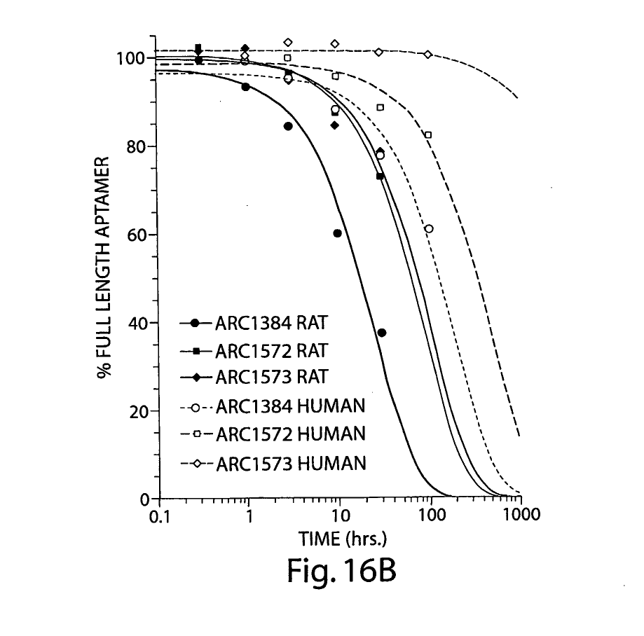

Sample data is shown in FIGS. 16A and 16B and the results for the aptamers tested are summarized in Table 29. Consistent with our expectations, aptamers are more stable in human plasma than in rat plasma and increasing the number of stabilizing modifications to the sugar-phosphate backbone correlates with increasing plasma stability.

TABLE 29
Plasma stability of ARC445 and ARC656 derivatives

| | | | ~T½ in |
|---|---|---|---|
| SEQ ID | | ~T½ in rat | human |
| NO | ARC # | plasma (hrs) | plasma (hrs) |
| 101 | ARC445 | 2.2 | Not done |
| 176 | ARC1335 | 16 | Not done |
| 178 | ARC1337 | 29 | Not done |
| 179 | ARC1382 | 16 | Not done |
| 180 | ARC1383 | 26 | Not done |
| 181 | ARC1384 | 22 | 149 |
| 182 | ARC1572 | 62 | 339 |
| 183 | ARC1573 | 74 | >500 |
| 216 | ARC1666 | 11 | 49 |
| 219 | ARC1729 | 42 | 180 |
| 157 | ARC656 | Not done | 6 |
| 285 | ARC1410 | Not done | 9 |

### Example 4B

### Pharmacokinetics of anti-IgE Aptamers Versus PEGylation

### ARC1785, ARC1787, ARC1788, and ARC1790 after IV Administration to Mice at 10 mg/kg

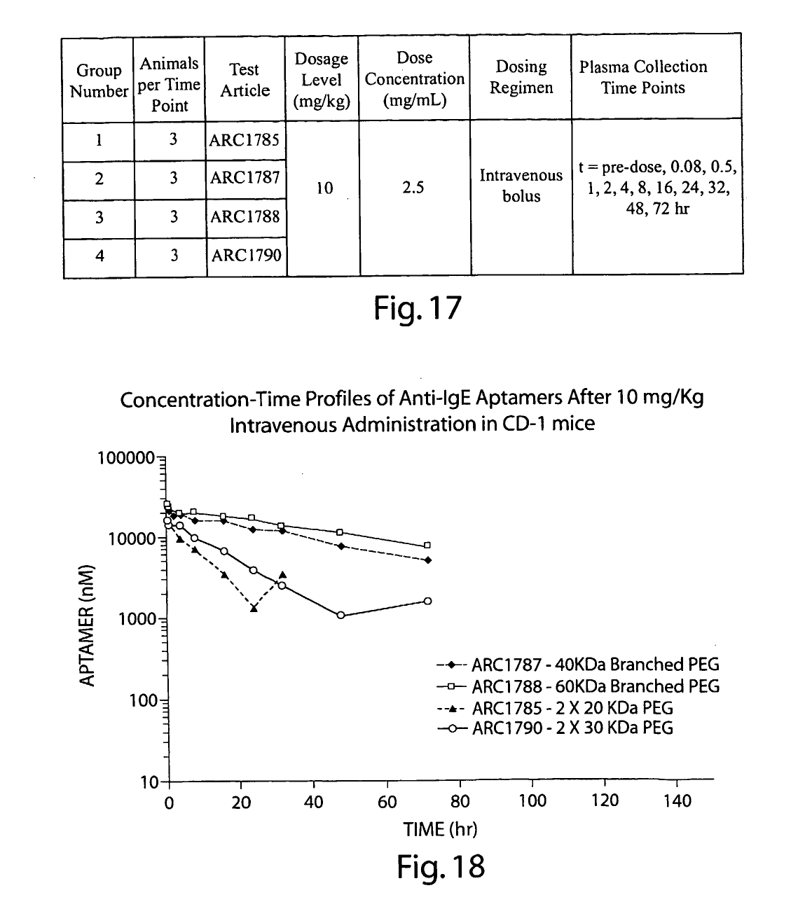

The design for the murine pharmacokinetic study of ARC1785, ARC1787, ARC1788, and ARC1790 via intravenous (IV) administration is shown in FIG. 17. Briefly, the study consisted of 3 groups (33 animals total). At each sample collection time point, 3 animals were terminally sacrificed and 0.5 mL of whole blood collected via cardiac puncture for harvesting of plasma. The aptamer was formulated for injection from a lyophilized powder to a final concentration of 10 mg/mL (oligo weight) in standard 0.9% saline and sterile-filtered (0.2 μm) prior to dosing. The route of administration used was a single intravenous bolus injection at a dose of 10 mg/kg. At specified time points, t=pre-dose, 0.08, 0.5, 1, 2, 4, 8, 16, 24, 32, 48, and 72 hours, whole blood samples were obtained, transferred directly to K2EDTA-coated tubes, mixed by inversion, and placed on wet ice.

Plasma was harvested by centrifugation of blood-EDTA tubes at 3500 rpm for 5 minutes. Plasma samples were transferred to fresh labeled 1.5 mL tubes and stored at −80° C. until the time of analysis. Analysis of plasma samples for aptamer concentration was accomplished using a homogeneous assay format utilizing the direct addition of plasma aliquots to assay wells containing the commercially available fluorescent nucleic acid detection reagent Oligreen™ (Molecular Probes, Eugene, Oreg.). After a brief incubation period (5 min) at room temperature, protected from light, the assay plates were read by a fluorescence plate reader. The fluorescence signal from each well was proportional to the concentration of aptamer in the well, and sample concentrations were calculated by interpolation of fluorescence values from a fluorescence-concentration standard curve (mean values from duplicate curves). Mean plasma concentrations were obtained at each time point from the three animals in each group, and are plotted versus time post-dose in FIG. 18.

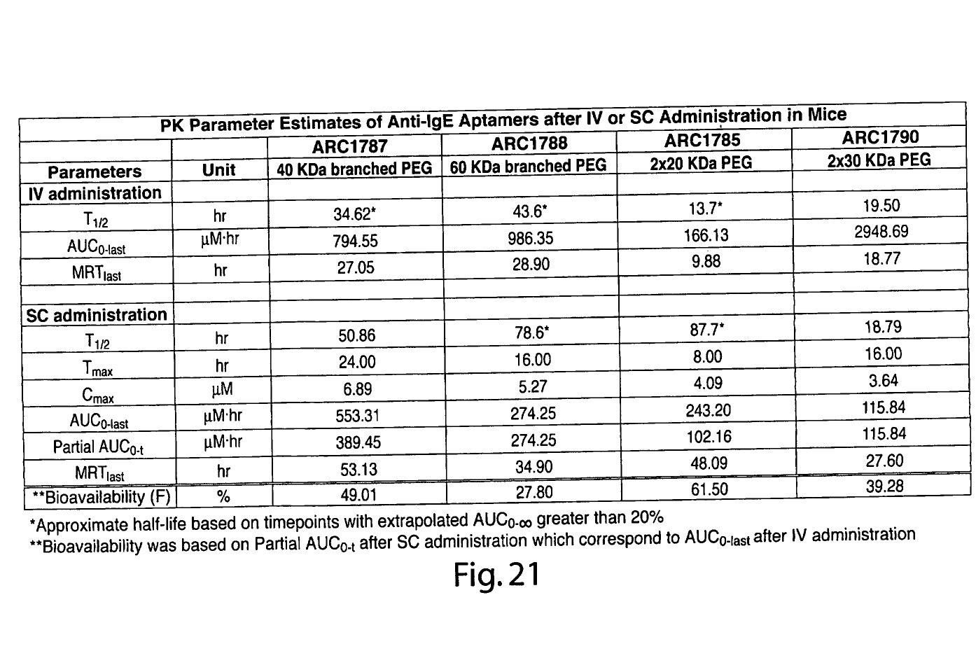

Plasma concentration versus time data appeared was subjected to noncompartmental analysis (NCA) using the industry standard pharmacokinetic modeling software WinNonLin™ v.4.0 (Pharsight Corp., Mountain View, Calif.). Estimates were obtained for the following primary pharmacokinetic parameters: maximum plasma concentration, Cmax; area under the concentration-time curve, AUC; terminal half-life, t1/2; terminal clearance, C1; and volume of distribution at steady state, Vss. Noncompartmental analysis (NCA) of the data using WinNonLin™ v.4.0 (Pharsight Corp., Mountain View, Calif.), yielded the estimates for the primary pharmacokinetic parameters listed in FIG. 21.

### Example 4C

### Pharmacokinetics and Bioavailability of Anti-IgE Aptamers Versus PEGylation

### ARC1785, ARC1787, ARC1788 and ARC1790 after SC Administration to Mice at 10 mg/kg

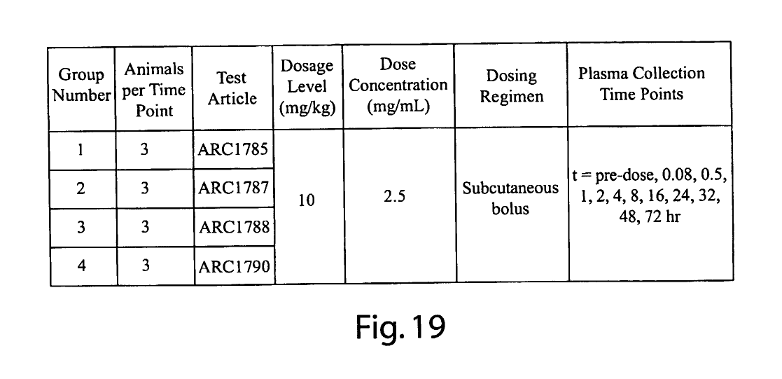

The design for the murine pharmacokinetic study of the bioavailability of ARC1785, ARC1787, ARC1788, and ARC1790 via subcutaneous routes of administration is shown in FIG. 19. Briefly, the study consisted of 3 groups (33 animals total). At each sample collection time point, 3 animals were terminally sacrificed and 0.5 mL of whole blood collected via cardiac puncture for harvesting of plasma. The aptamer was formulated for injection from a lyophilized powder to a final concentration of 10 mg/mL (oligo weight) in standard 0.9% saline and sterile-filtered (0.2 μm) prior to dosing. The route of administration used was a single subcutaneous bolus injection at a dose of 10 mg/kg. At specified time points, t=pre-dose, 0.08, 0.5, 1, 2, 4, 8, 16, 24, 32, 48, and 72 hours, whole blood samples were obtained, transferred directly to K2EDTA-coated tubes, mixed by inversion, and placed on wet ice.

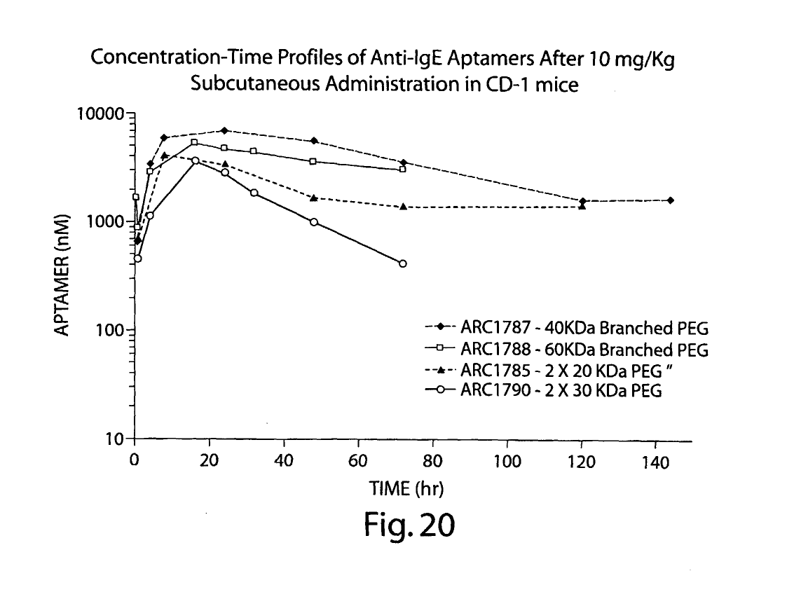

Plasma was harvested by centrifugation of blood-EDTA tubes at 3500 rpm for 5 minutes. Plasma samples were transferred to fresh labeled 1.5 mL tubes and stored at −80° C. until the time of analysis. Analysis of plasma samples for aptamer concentration was accomplished using a homogeneous assay format utilizing the direct addition of plasma aliquots to assay wells containing the commercially available fluorescent nucleic acid detection reagent Oligreen™ (Molecular Probes, Eugene, Oreg.). After a brief incubation period (5 min) at room temperature, protected from light, the assay plates were read by a fluorescence plate reader. The fluorescence signal from each well was proportional to the concentration of aptamer in the well, and sample concentrations were calculated by interpolation of fluorescence values from a fluorescence-concentration standard curve (mean values from duplicate curves). Mean plasma concentrations were obtained at each time point from the three animals in each group, and are plotted versus time post-dose in FIG. 20.

Plasma concentration versus time data appeared was subjected to noncompartmental analysis (NCA) using the industry standard pharmacokinetic modeling software WinNonLin™ v.4.0. Estimates were obtained for the following primary pharmacokinetic parameters: maximum plasma concentration, Cmax; area under the concentration-time curve, AUC; terminal half-life, t1/2; terminal clearance, C1; and volume of distribution at steady state, Vss. Noncompartmental analysis (NCA) of the data using WinNonLin™ v.4.0 (Pharsight Corp., Mountain View, Calif.)., yielded the estimates for the primary pharmacokinetic parameters listed in FIG. 21. As can be seen from the table of FIG. 21, all of the PEG conjugated aptamers tested exhibited subcutaneous bioavailability.

### Example 4D

### Ex Vivo Pharmacodynamic Study of PEGylated Anti-IgE Aptamers in Cynomolgus Macaque

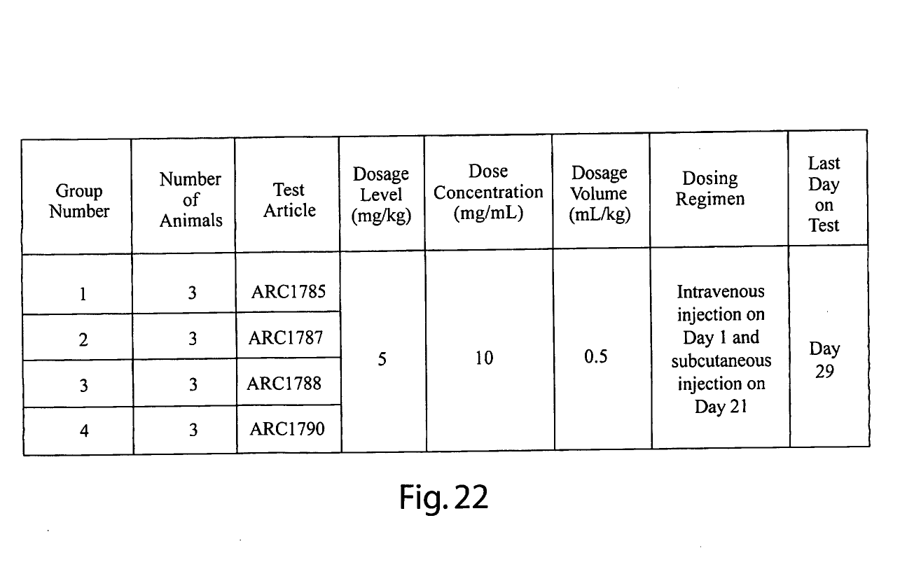

The design for the ex vivo study of the pharmacodynamics of ARC1785, ARC1787, ARC1788, and ARC1790 via subcutaneous and intravenous routes of administration in cynomolgus macaques is shown in FIG. 22. Briefly, the study consisted of 4 groups (12 animals total). At each sample collection time point, approximately 1.8 mL of whole blood was collected via peripheral vessel for harvesting of plasma. The aptamer was formulated for injection from a lyophilized powder to a final concentration of 10 mg/mL (oligo weight) in standard 0.9% saline and sterile-filtered (0.2 μm) prior to dosing. The route of administration used was a single intravenous injection on Day I at a dose of 5 mg/kg, and a subcutaneous injection on Day 21 at a dose of 5 mg/kg. At specified time points, t=pre-dose, 5 and 30 minutes, 1, 2, 4, 6, 8, 12, 24, 36, 48, 72, 96, 144, 192, 240, 288, and 336 hours post treatment, whole blood samples were obtained, transferred directly to K2EDTA-coated tubes, mixed by inversion, and placed on wet ice.

Plasma was harvested by centrifugation of blood-EDTA tubes at 3500 rpm for 5 minutes. Plasma samples were transferred to fresh labeled 1.5 mL tubes and stored at −80° C. until the time of analysis. Analysis of plasma samples for aptamer activity was accomplished using an ELISA based assay measuring the ability of IgE aptamers to inhibit binding to FcεRI_{α}.

Harvested plasma containing ARC1666 was tested for the ability to inhibit complex formation between h-IgE (Athens Research, Athens Ga.) and soluble FcεR1_{α—}Fc (purified in-house) using an ELISA assay as follows. FcεR1 (100 μL, 0.5 μg/mL) in 1×PBS was incubated in the wells of a Nunc Maxisorb 96 well plate overnight at 4° C. to coat the surface of the wells. The supernatants were removed, the wells were washed 3 times with 200 μl 1×TBS+0.05% Tween-20, and the wells were blocked with 200 μL 5% milk in 1×TBS plus 0.05% Tween-20 at room temperature for one hour. After blocking, the wells were washed three times with 1×TBS+0.05% Tween-20.

A dilution of ARC1666 in cynomolgus monkey sera from each time point (typically a 1:1.5 dilution) was next incubated with 2.5 nM h-IgE in 100 μL TBS plus 0.05% Tween-20 at room temperature for 15 minutes, and then the mixtures were added to the assay wells and incubated at room temperature for 1 hour. The wells were then washed 3 times with 200 μL 1×TBS+0.05% Tween-20. Bound h-IgE was detected by the addition of HRP-labeled anti-h-IgE polyclonal antibody (Goat anti-human IgE-HRP (074-1004) (KPL, Gaithersburg, Md.)). After forty-five minute incubation with anti-h-IgE polyclonal antibody, wells were washed 3 times with 200 μL 1×TBS+0.05% Tween-20. Quantablue substrate (Pierce, Rockford, Ill.) was used to detect peroxidase activity. 100 μl of Quantablue substrate was added to each well and incubated at room temperature for 1 minute. Next, 100 μl of the provided stop solution was added to each well, and the plates were read on a VersaMax 96 well plate reader at 450 nm.

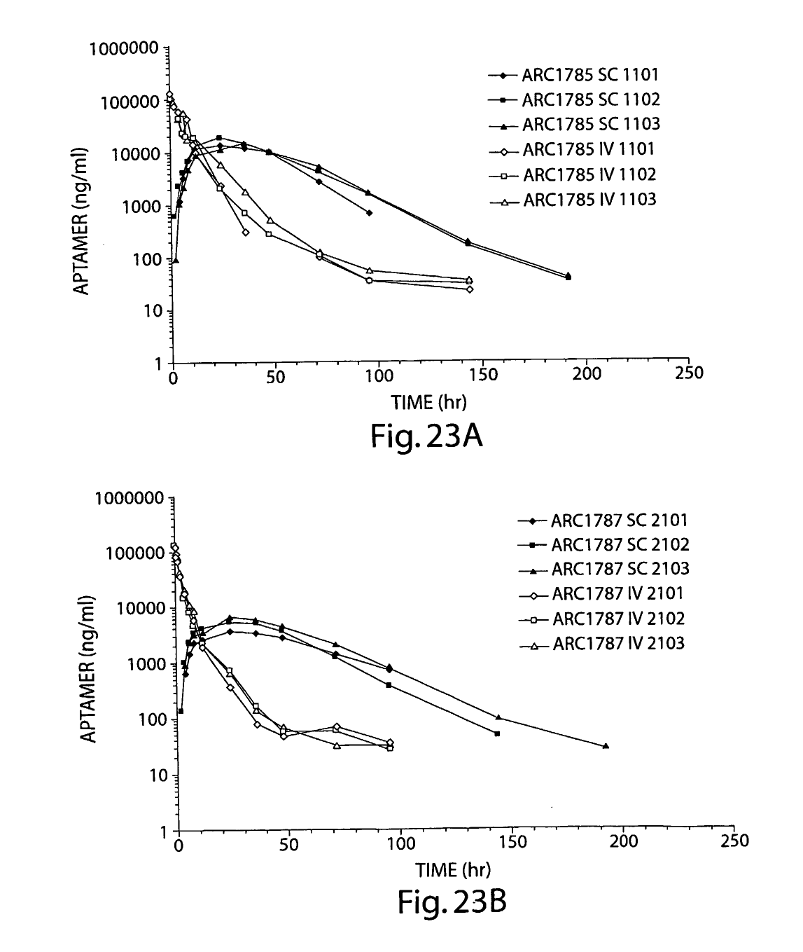

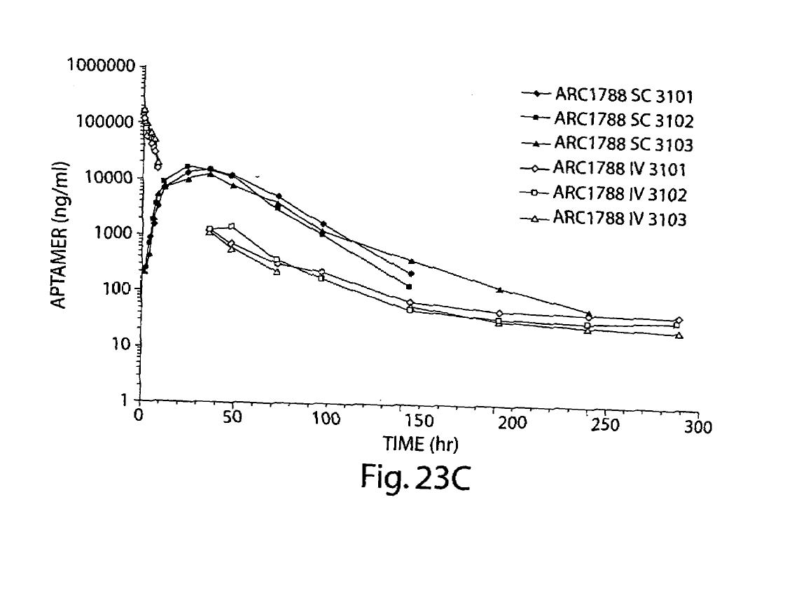

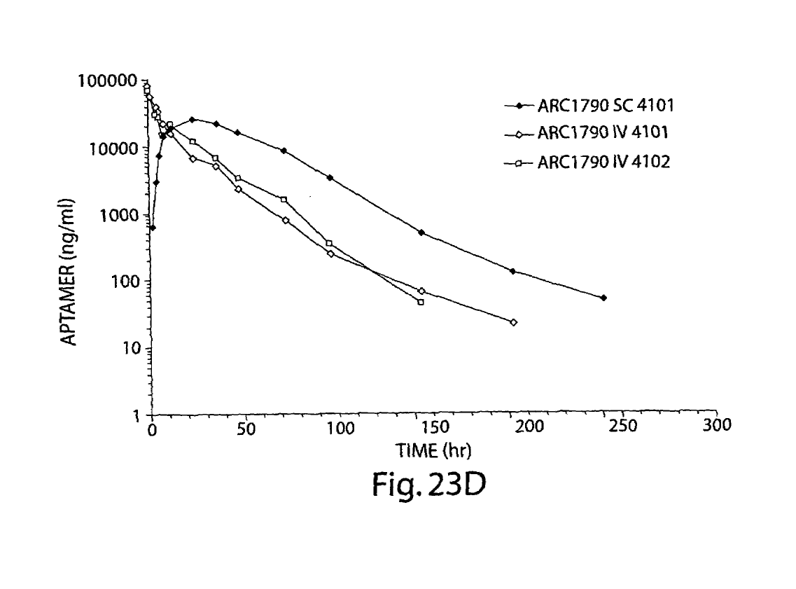

Aptamer concentration in each sample was determined from a standard curve based on percent inhibition in the ELISA for each aptamer as follows. For the standard curve, ARC1666 was serially diluted (1:5) in a percentage of cynomolgus monkey sera (0, 3.9 nM-45 nM) and run in the receptor (FcεR1) binding inhibition ELISA described above in Example 3B. The final percentage of cynomolgus monkey sera used to generate the standard curve depended on the dilution factor of the samples (typically 1:15, described above) to normalize for percentage of monkey sera used in both the standard curve and samples. Absorbance values at 450 nm (“ABS”) versus log aptamer concentration was graphed and a linear curve fit (y=m×+b) was completed where “y” is the measured absorbance value, at 450 nm, “m” is the slope (provided by the curve fit), “b” is the y-intercept (provided by the curve fit), and “x” is the amount of functional aptamer, to be calculated. The following equation was used to calculate the final amount of active aptamer at the various time points: Amount of functional aptamer [nM]=[[10̂((ABS-b)/(m))]*dilution factor]. Active aptamer concentration versus time data for each PEGylated aptamer tested is shown in FIG. 23.

The invention having now been described by way of written description and example, those of skill in the art will recognize that the invention can be practiced in a variety of embodiments and that the description and examples above are for purposes of illustration and not limitation of the following claims.

1) An aptamer that binds to IgE comprising a nucleic acid sequence having a 3′ and 5′ end selected from the group consisting of SEQ ID NOs 299-336.

2) The aptamer of claim 1, wherein the nucleic acid sequence is further modified to comprise at least one chemical modification.

3) The aptamer of claim 2, wherein the modification is selected from the group consisting of a chemical substitution at sugar position; a chemical substitution at a phosphate position; and a chemical substitution at a base position of the nucleic acid.

4) The aptamer of claim 2, wherein the modification is selected from the group consisting of: incorporation of a modified nucleotide, 3′ capping, conjugation to a high molecular weight non-immunogenic compound, and conjugation to a liphophilic compound.

5) The aptamer of claim 4, wherein the high molecular weight, non-immunogenic compound is polyalkylene glycol.

6) The aptamer of claim 5, wherein the polyalkylene glycol is polyethylene glycol.

7) The aptamer of claim 6, wherein the nucleic acid sequence comprises a polyethylene glycol conjugated to its 5′ end.

8) The aptamer of claim 6, wherein the nucleic acid sequence comprises a polyethylene glycol conjugated to its 3′ end.

9) The aptamer of claim 4, wherein the 3′ cap is an inverted nucleotide.

10) The aptamer of claim 5, wherein the polyethylene glycol comprises a molecular weight selected from the group consisting of: a 60 kDa, a 40 kDa, a 30 kDa and a 20 kDa.

11) A composition comprising a therapeutically effective amount of the aptamer of claim 1 or a salt thereof and a pharmaceutically acceptable carrier or diluent.

12) A method of treating atopic disease comprising administering an aptamer to a subject having atopic disease an effective amount of the aptamer of claim 1 or a salt thereof.

13) The method according to claim 12, wherein the aptamer is selected from the group consisting of SEQ ID NOs 299-336.

14) A method of treating a disease mediated by IgE comprising administering an effective amount of the aptamer or salt thereof according to claim 1 to a patient in need thereof.

15) A method of detecting IgE in a sample comprising the steps of contacting the sample with the aptamer of claim 1, and determining whether the aptamer specifically binds to the sample, wherein in binding of the aptamer to the sample indicates the sample contains IgE.

16) An aptamer that binds an antibody.

17) The aptamer, as claimed in claim 16, wherein said antibody comprises a constant (Fc) region.

18) The aptamer, as claimed in claim 17, wherein said aptamer binds said Fc region of said antibody.

19) The aptamer, as claimed in claim 18, wherein said antibody is selected from the group consisting of: IgA, IgD, IgE, IgG, and IgM.

20) An aptamer which binds the Fc portion of an antibody, wherein, said aptamer is produced according to the method comprising:
a) preparing a candidate mixture of nucleic acids;
b) contacting said candidate mixture of nucleic acids with an antibody comprising an Fc region, wherein, nucleic acids having an increased affinity to said antibody relative to the candidate mixture may be partitioned from the remainder of the candidate mixture;
c) partitioning the increased affinity nucleic acids from the remainder of the candidate mixture;
d) amplifying the increased affinity nucleic acids to yield a mixture of nucleic acids enriched for nucleic acids with relatively higher affinity and specificity for binding to said antibody; and
e) determining whether said nucleic acids with relatively higher affinity and specificity for binding to said antibody, from step d, bind the Fc region.

21) The aptamer, as claimed in claim 20, wherein the method of determining whether said nucleic acids with relatively higher affinity and specificity for binding to said antibody, from step d, binds the Fc region further comprises the following steps:
f) contacting said nucleic acids with relatively higher affinity and specificity for binding to said antibody, from step d, with the antibody, from step b, under conditions such that nucleic acid-antibody complexes are formed; and
g) contacting said nucleic acid-antibody complex with the Fc receptor for said antibody under conditions such that nucleic acid-antibody complexes which do not bind said Fc receptor are isolated.

22) The aptamer, as claimed in claim 21, further comprising the following step:
h) dissociating the nucleic acid ligand from said isolated nucleic acid-antibody complex, thereby, producing a purified aptamer that binds said antibody under conditions such that said aptamer bound antibody fails to bind the Fc receptor specific for said antibody.

23) The aptamer, as claimed in claim 20, wherein said antibody is selected from the group consisting of: IgA, IgD, IgE, IgG, and IgM.

24) The aptamer, as claimed in claim 20, wherein said Fc receptor is isolated from the cell and solubilized.

25) The aptamer, as claimed in claim 20, wherein said Fc receptor is in situ in a cell.

26) A method for identifying aptamers that bind the Fc portion of an antibody comprising:
a) preparing a candidate mixture of nucleic acids;
b) contacting said candidate mixture of nucleic acids with an antibody comprising an Fc region, wherein, nucleic acids having an increased affinity to said antibody relative to the candidate mixture may be partitioned from the remainder of the candidate mixture;
c) partitioning the increased affinity nucleic acids from the remainder of the candidate mixture;
d) amplifying the increased affinity nucleic acids to yield a mixture of nucleic acids enriched for nucleic acids with relatively higher affinity and specificity for binding to said antibody; and
e) determining whether said nucleic acids with relatively higher affinity and specificity for binding to said antibody, from step d, bind the Fc region.

27) The method, as claimed in claim 26, wherein the method of determining whether said nucleic acids with relatively higher affinity and specificity for binding to said antibody, from step d, binds the Fc region further comprises the following steps:
f) contacting said nucleic acids with relatively higher affinity and specificity for binding to said antibody, from step d, with the antibody, from step b, under conditions such that nucleic acid-antibody complexes are formed; and
g) contacting said nucleic acid-antibody complex with the Fc receptor for said antibody under conditions such that nucleic acid-antibody complexes which do not bind said Fc receptor are identified.

28) The method, as claimed in claim 27, further comprising:
h) dissociating the nucleic acid ligand from said isolated nucleic acid-antibody complex, thereby, identifying a substantially purified aptamer that binds said antibody under conditions such that said aptamer bound antibody fails to bind the Fc receptor specific for said antibody.

29) The method, as claimed in claim 26, wherein said antibody is selected from the group consisting of: IgA, IgD, IgE, IgG, and IgM.

30) The method, as claimed in claim 26, wherein said Fc receptor is isolated from the cell and solubilized.

31) The method, as claimed in claim 26, where said Fc receptor is in situ in a cell.

32) A method for purifying antibodies in a sample comprising:
a) providing a sample having a first antibody concentration;
b) providing an aptamer that binds an antibody;
c) contacting said sample with said aptamer under conditions such that said aptamer binds with said antibody, thereby, forming an aptamer antibody complex; and
d) partitioning said aptamer antibody complex from said sample, thereby, creating a purified second sample wherein the concentration of antibody in said second sample is higher than the concentration of antibody in said first sample.

33) The method, as claimed in claim 32, wherein said antibody comprises a constant (Fc) region.

34) The method, as claimed in claim 33, wherein said aptamer binds said Fc region of said antibody.

35) The method, as claimed in claim 34, wherein said Fc region is the Fc region of an antibody selected from the group consisting of: IgA, IgD, IgE, IgG, and IgM.


SEQUENCE LISTING (336 sequences)

SEQ ID NO: 1 - DNA, 93 bp, artificial
  synthetic template
catcgatgctagtcgtaacgatccnnnnnnnnnnnnnnnnnnnnnnnnnnnnnncgagaacgttctctcctctccctatagtgagtcgtatta

SEQ ID NO: 2 - DNA, 92 bp, artificial
  synthetic template
catgcatcgcgactgactagccgnnnnnnnnnnnnnnnnnnnnnnnnnnnnnngtagaacgttctctcctctccctatagtgagtcgtatta

SEQ ID NO: 3 - DNA, 92 bp, artificial
  synthetic template
catcgatcgatcgatcgacagcgnnnnnnnnnnnnnnnnnnnnnnnnnnnnnngtagaacgttctctcctctccctatagtgagtcgtatta

SEQ ID NO: 4 - DNA, 10 bp, artificial
  synthetic immunostimulatory motif
aacgttcgag

SEQ ID NO: 5 - DNA, 88 bp, artificial
  synthetic template
gggaaaagcgaatcatacacaagannnnnnnnnnnnnnnnnnnnnnnnnnnnnnnnnnnnnnnngctccgccagagaccaaccgagaa

SEQ ID NO: 6 - DNA, 41 bp, artificial
  synthetic primer
taatacgactcactatagggaaaagcgaatcatacacaaga

SEQ ID NO: 7 - DNA, 24 bp, artificial
  synthetic primer
ttctcggttggtctctggcggagc

SEQ ID NO: 8 - RNA, 88 nt, artificial
  synthetic template
gggaaaagcgaaucauacacaagannnnnnnnnnnnnnnnnnnnnnnnnnnnnnnnnnnnnnnngcuccgccagagaccaaccgagaa

SEQ ID NO: 9 - RNA, 24 nt, artificial
  synthetic fixed region
gggaaaagcgaaucauacacaaga

SEQ ID NO: 10 - RNA, 24 nt, artificial
  synthetic fixed region
gcuccgccagagaccaaccgagaa

SEQ ID NO: 11 - RNA, 88 nt, artificial
  synthetic aptamer
gggaaaagcgaaucauacacaagacgucgccagauugagugucgugguucggguugaggcggaagcuccgccagagaccaaccgagaa

SEQ ID NO: 12 - RNA, 87 nt, artificial
  synthetic aptamer
ggaaaagcgaaucauacacaagagucgcgauagauugcuugugaaugguuuugguggaagcgggcuccgccagagaccaaccgagaa

SEQ ID NO: 13 - RNA, 88 nt, artificial
  synthetic aptamer
gggaaaagcgaaucauacacaagagucgcuagauugcuaguguaugguuuaucuaaaggcggccgcuccgccagagaccaaccgagaa

SEQ ID NO: 14 - RNA, 87 nt, artificial
  synthetic aptamer
gggaaaagcgaaucauacacaagaggucuuacagauccuguguagugguucgauacaugcggggcuccgccagagaccaaccgagaa

SEQ ID NO: 15 - RNA, 88 nt, artificial
  synthetic aptamer
gggaaaagcgaaucauacacaagacgugagcauaucauugaguguagugguuccggaguaagucgcuccgccagagaccaaccgagaa

SEQ ID NO: 16 - RNA, 87 nt, artificial
  synthetic aptamer
gggaaaagcgaaucauacacaagagcaccuugacugugauucgcgggugugagucgugcgaaggcuccgccagagaccaaccgagaa

SEQ ID NO: 17 - RNA, 88 nt, artificial
  synthetic aptamer
gggaaaagcgaaucauacacaagagugcaagaagugcauugcugugucugguucuuggcgaugugcuccgccagagaccaaccgagaa

SEQ ID NO: 18 - RNA, 88 nt, artificial
  synthetic aptamer
gggaaaagcgaaucauacacaagauccgagggugggcaauaggcucacaaggguuucgcgugaugcuccgccagagaccaaccgagaa

SEQ ID NO: 19 - RNA, 88 nt, artificial
  synthetic aptamer
gggaaaagcgaaucauacacaagagugccgaggcauugcuugguaugguuccggucuugucggggcuccgccagagaccaaccgagaa

SEQ ID NO: 20 - RNA, 88 nt, artificial
  synthetic aptamer
gggaaaagcgaaucauacacaagacgucgccagauugaguguggugguucggguugaggcggaagcuccgccagagaccaaccgagaa

SEQ ID NO: 21 - RNA, 90 nt, artificial
  synthetic aptamer
gggaaaagcgaaucauacacaagacgucaguaagauugaguguaugguuccugguggacaauaauggcuccgccagagaccaaccgagaa

SEQ ID NO: 22 - RNA, 88 nt, artificial
  synthetic aptamer
gggaaaagcgaaucauacacaagagaguggaggagguauguaugguuugugcgucuggugcggugcuccgccagagaccaaccgagaa

SEQ ID NO: 23 - DNA, 75 bp, artificial
  synthetic template
gggagaggagagaacgttctacnnnnnnnnnnnnnnnnnnnnnnnnnnnnnncgctgtcgatcgatcgatcgatg

SEQ ID NO: 24 - DNA, 22 bp, artificial
  synthetic primer
gggagaggagagaacgttctac

SEQ ID NO: 25 - DNA, 22 bp, artificial
  synthetic primer
catcgatcgatcgatcgacagc

SEQ ID NO: 26 - RNA, 22 nt, artificial
  synthetic fixed region
gggagaggagagaacguucuac

SEQ ID NO: 27 - RNA, 23 nt, artificial
  synthetic fixed region
cgcugucgaucgaucgaucgaug

SEQ ID NO: 28 - DNA, 75 bp, artificial
  synthetic aptamer
gggagaggagagaacguucuacgauuagcagggagggagagugcgaagaggacgcugucgaucgaucgaucgaug

SEQ ID NO: 29 - DNA, 75 bp, artificial
  synthetic aptamer
gggagaggagagaacguucuacacucuggggacccgugggggagugcagcaacgcugucgaucgaucgaucgaug

SEQ ID NO: 30 - DNA, 74 bp, artificial
  synthetic aptamer
gggagaggagagaacguucuacgaggugagggucuacaauggagggauggucgcugucgaucgaucgaucgaug

SEQ ID NO: 31 - DNA, 75 bp, artificial
  synthetic aptamer
gggagaggagagaacguucuacccgcagcauagccugnggacccaugnggggcgcugucgaucgaucgaucgaug

SEQ ID NO: 32 - DNA, 75 bp, artificial
  synthetic aptamer
gggagaggagagaacguucuacuggggggcguguucauuagcagcgucgugucgcugucgaucgaucgaucgaug

SEQ ID NO: 33 - DNA, 75 bp, artificial
  synthetic aptamer
gggagaggagagaacguucuacgcagcgcaucuggggacccaagaggggauucgcugucgaucgaucgaucgaug

SEQ ID NO: 34 - DNA, 73 bp, artificial
  synthetic aptamer
gggagaggagagaacguucuacgggauggguaguuggauggaaaugggaacgcugucgaucgaucgaucgaug

SEQ ID NO: 35 - DNA, 74 bp, artificial
  synthetic aptamer
gggagaggagagaacguucuacgagguguagggauagagggguguagguaacgcugucgaucgaucgaucgaug

SEQ ID NO: 36 - DNA, 75 bp, artificial
  synthetic aptamer
gggagaggagagaacguucuacaggaguggagcuacagagaggguuaggggucgcugucgaucgaucgaucgaug

SEQ ID NO: 37 - DNA, 75 bp, artificial
  synthetic aptamer
gggagaggagagaacguucuacggauguugggagugauagaaggaaggggagcgcugucgaucgaucgaucgaug

SEQ ID NO: 38 - DNA, 76 bp, artificial
  synthetic aptamer
gggagaggagagaacguucuacuugggguggaaggaguaagggaggugcugaucgcugucgaucgaucgaucgaug

SEQ ID NO: 39 - DNA, 75 bp, artificial
  synthetic aptamer
gggagaggagagaacguucuacguauuaggggggaaggggaggaauagaucacgcugucgaucgaucgaucgaug

SEQ ID NO: 40 - DNA, 76 bp, artificial
  synthetic aptamer
gggagaggagagaacguucuacagggagagaguguugagugaagaggaggagucgcugucgaucgaucgaucgaug

SEQ ID NO: 41 - DNA, 75 bp, artificial
  synthetic aptamer
gggagaggagagaacguucuacauugugcuccuggggcccaguggggagccacgcugucgaucgaucgaucgaug

SEQ ID NO: 42 - DNA, 75 bp, artificial
  synthetic aptamer
gggagaggagagaacguucuacgagcagcccuggggcccggagggggauggucgcugucgaucgaucgaucgaug

SEQ ID NO: 43 - DNA, 75 bp, artificial
  synthetic aptamer
gggagaggagagaacguucuacaggcaguucuggggacccaugggggaagugcgcugucgaucgaucgaucgaug

SEQ ID NO: 44 - DNA, 75 bp, artificial
  synthetic aptamer
gggagaggagagaacguucuaccaacggcauccugggccccacaggggaugucgcugucgaucgaucgaucgaug

SEQ ID NO: 45 - DNA, 74 bp, artificial
  synthetic aptamer
gggagaggagagaacguucuacgaguggauagggaagaaggggaguagucacgcugucgaucgaucgaucgaug

SEQ ID NO: 46 - DNA, 75 bp, artificial
  synthetic aptamer
gggagaggagagaacguucuacccgcagcauagccuggggacccauggggggcgcugucgaucgaucgaucgaug

SEQ ID NO: 47 - DNA, 76 bp, artificial
  synthetic aptamer
gggagaggagagaacguucuacggucgcgugugggggacggauggguauuggucgcugucnaucgaucgaucgaug

SEQ ID NO: 48 - DNA, 75 bp, artificial
  synthetic aptamer
gggagaggagagaacguucuacgggguuacgucgcacgauacaugcauucaucgcugucgaucgaucgaucgaug

SEQ ID NO: 49 - DNA, 75 bp, artificial
  synthetic aptamer
gggagaggagagaacguucuacuagcgaggagggguuuucuauuuuugcgaucgcugucgaucgaucgaucgaug

SEQ ID NO: 50 - DNA, 75 bp, artificial
  synthetic aptamer
gggagaggagagaacguucuacaagcaguucuggggacccaugggggaagugcgcugucgaucgaucgaucgaug

SEQ ID NO: 51 - DNA, 75 bp, artificial; identical to SEQ ID NO: 23
  synthetic template

SEQ ID NO: 52 - DNA, 22 bp, artificial
  synthetic primer
gggagaggagagaacgttctac

SEQ ID NO: 53 - DNA, 22 bp, artificial
  synthetic primer
catcgatcgatcgatcgacagc

SEQ ID NO: 54 - RNA, 21 nt, artificial
  synthetic fixed region
gggagaggagagaacguucua

SEQ ID NO: 55 - RNA, 23 nt, artificial
  synthetic fixed region
cgcugucgaucgaucgaucgaug

SEQ ID NO: 56 - RNA, 82 nt, artificial
  synthetic aptamer
gggagaggagagaacguucuacgaucugggcgagccagucugacugaggaagcgcugucgaucgaucgaucgaugaagggcg

SEQ ID NO: 57 - RNA, 82 nt, artificial
  synthetic aptamer
gggagaggagagaacguucuacgcggucggguguguggaggaaguaguucgucgcugucgaucgaucgaucgaugaagggcg

SEQ ID NO: 58 - RNA, 82 nt, artificial
  synthetic aptamer
gggagaggagagaacguucuacgacguuaaugcagcggcuagggaugggcagcgcugucgaucgaucgaucgaugaagggcg

SEQ ID NO: 59 - RNA, 82 nt, artificial
  synthetic aptamer
gggagaggagagaacguucuacaggcguguugguaggguacgacgaggcaugcgcugucgaucgaucgaucgaugaagggcg

SEQ ID NO: 60 - RNA, 82 nt, artificial
  synthetic aptamer
gggagaggagagaacguucuacugagggauaauacgggugggauugucuucccgcugucgaucgaucgaucgaugaagggcg

SEQ ID NO: 61 - RNA, 82 nt, artificial
  synthetic aptamer
gggagaggagagaacguucuacgaaaaagauaugagagaaaggauuaagagacgcugucgaucgaucgaucgaugaagggcg

SEQ ID NO: 62 - RNA, 82 nt, artificial
  synthetic aptamer
gggagaggagagaacguucuacgaagaagauaugagagaaaggauuaagagacgcugucgaucgaucgaucgaugaagggcg

SEQ ID NO: 63 - RNA, 82 nt, artificial; identical to SEQ ID NO: 61
  synthetic aptamer

SEQ ID NO: 64 - RNA, 82 nt, artificial
  synthetic aptamer
gggagaggagagaacguucuacgaaaaagauaugagagaaaggauuaagaggcgcugucgaucgaucgaucgaugaagggcg

SEQ ID NO: 65 - RNA, 82 nt, artificial
  synthetic aptamer
gggagaggagagaacguucuacgaaaaagacaugagagaaaggauuaagagacgcugucgaucgaucgaucgaugaagggcg

SEQ ID NO: 66 - RNA, 83 nt, artificial
  synthetic aptamer
gggagaggagagaacguucuacnaaaaaguauaugagagaaaggauuaanagacgcugucgaucgaucgaucgaugaagggcg

SEQ ID NO: 67 - RNA, 83 nt, artificial
  synthetic aptamer
gggagaggagagaacguucuacgaaaaagauaugagagaaaaggauugagagaugcugucgaucgaucgaucgaugaagggcg

SEQ ID NO: 68 - RNA, 83 nt, artificial
  synthetic aptamer
gggagaggagagcacguucuacgaaaaagauauggagagaaaggauuaagagacgcugucgaucgaucgaucgaugaagggcg

SEQ ID NO: 69 - RNA, 84 nt, artificial
  synthetic aptamer
gggagaggagagaacguucuacgaaaaagauaugagagaaaggauuaaaagagacgcugucgaucgaucgaucgaugaagggcg

SEQ ID NO: 70 - RNA, 85 nt, artificial
  synthetic aptamer
gggagaggagagaacguucuacgaanaagauacauaguagaaaggauuaauaagacgcugucgaucgaucgaucgaugaagggcg

SEQ ID NO: 71 - RNA, 82 nt, artificial; identical to SEQ ID NO: 59
  synthetic aptamer

SEQ ID NO: 72 - RNA, 82 nt, artificial
  synthetic aptamer
gggagaggagagaacguucuacgcaaaaaugugaugcgagguaauggaacgccgcugucgaucgaucgaucgaugaagggcg

SEQ ID NO: 73 - RNA, 82 nt, artificial
  synthetic aptamer
gggagaggagagaacguucuacggaccucagcgauagggguugaaaccgacacgcugucgaucgaucgaucgaugaagggcg

SEQ ID NO: 74 - RNA, 82 nt, artificial
  synthetic aptamer
gggagaggagagaacguucuacauggucggaugcuggggaguaggcaagguucgcugucgaucgaucgaucgaugaagggcg

SEQ ID NO: 75 - RNA, 82 nt, artificial
  synthetic aptamer
gggagaggagagaacguucuacguaucggcgagcgaagcauccgggagcguucgcugucgaucgaucgaucgaugaagggcg

SEQ ID NO: 76 - RNA, 82 nt, artificial
  synthetic aptamer
gggagaggagagaacguucuacguauuggcgcgcgaagcauccgggagcguucgcugucgaucgaucgaucgaugaagggcg

SEQ ID NO: 77 - RNA, 82 nt, artificial
  synthetic aptamer
gggagaggagagaacguucuacuuauaccugacggccggaggcgcauaggugcgcugucgaucgaucgaucgaugaagggcg

SEQ ID NO: 78 - RNA, 82 nt, artificial; identical to SEQ ID NO: 74
  synthetic aptamer

SEQ ID NO: 79 - RNA, 82 nt, artificial
  synthetic aptamer
gggagaggagagaacguucuacacgagaguacugaggcgcuugguacagagucgcugucgaucgaucgaucgaugaagggcg

SEQ ID NO: 80 - RNA, 82 nt, artificial
  synthetic aptamer
gggagaggagagaacguucuacagaagguagaaaaaggauagcugugagaagcgcugucgaucgaucgaucgaugaagggcg

SEQ ID NO: 81 - RNA, 82 nt, artificial; identical to SEQ ID NO: 60
  synthetic aptamer

SEQ ID NO: 82 - RNA, 84 nt, artificial
  synthetic aptamer
gggagaggagagaacguucuacauugagcguugaaguuggggaagcuccgaggccgcugucgaucgaucgaucgaugaagggcg

SEQ ID NO: 83 - RNA, 82 nt, artificial
  synthetic aptamer
gggagaggagagaacguucuacgcggagauauacagcgagguaauggaacgccgcugucgaucgaucgaucgaugaagggcg

SEQ ID NO: 84 - RNA, 82 nt, artificial
  synthetic aptamer
gggagaggagagaacguucuacgaagacagcccaauagcggcacggaacuugcgcugucgaucgaucgaucgaugaagggcg

SEQ ID NO: 85 - RNA, 84 nt, artificial
  synthetic aptamer
gggagaggagagaacguucuaccgguugagggcucgcguggaagggccaacacgcgcugucgaucgaucgaucgaugaagggcg

SEQ ID NO: 86 - RNA, 82 nt, artificial
  synthetic aptamer
gggagaggagagaacguucuacauaucaauagacucuugacguuuggguuugcgcugucgaucgaucgaucgaugaagggcg

SEQ ID NO: 87 - RNA, 79 nt, artificial
  synthetic aptamer
gggagaggagagaacguucuacagugaaggaaaaguaagugaaggugugcgcugucgaucgaucgaucgaugaagggcg

SEQ ID NO: 88 - RNA, 82 nt, artificial
  synthetic aptamer
gggagaggagagaacguucuacggaugaaaugagugucugcgauagguuaagcgcugucgaucgaucgaucgaugaagggcg

SEQ ID NO: 89 - RNA, 83 nt, artificial
  synthetic aptamer
gggagaggagagaacguucuacggaaggaaaugugugucugcgauagguuaagcgcugucgaucgaucgaucgaugaagggcg

SEQ ID NO: 90 - RNA, 49 nt, artificial
  synthetic aptamer
gggaaaagcgaaucauacacaagacgucgccagauugagugucgugguu

SEQ ID NO: 91 - RNA, 43 nt, artificial
  synthetic aptamer
ggaaucauacacaagacgucgccagauugagugucgugguucc

SEQ ID NO: 92 - RNA, 41 nt, artificial
  synthetic aptamer
ggaaucauacacaagacgucgccagauugagugucgugguu

SEQ ID NO: 93 - RNA, 37 nt, artificial
  synthetic aptamer
ggagauccgagggugggcaauaggcucacaaggguuu

SEQ ID NO: 94 - RNA, 35 nt, artificial
  synthetic aptamer
ggauccgagggugggcaauaggcucacaagggucc

SEQ ID NO: 95 - RNA, 43 nt, artificial
  synthetic aptamer
ggaaucauacacaagacgucaguaagauugaguguaugguucc

SEQ ID NO: 96 - RNA, 41 nt, artificial
  synthetic aptamer
ggaaucauacacaagacgucaguaagauugaguguaugguu

SEQ ID NO: 97 - DNA, 21 bp, artificial
  synthetic aptamer
uucuggggacccaugggggaa

SEQ ID NO: 98 - DNA, 23 bp, artificial
  synthetic aptamer
guucuggggacccaugggggaac

SEQ ID NO: 99 - DNA, 25 bp, artificial
  synthetic aptamer
aguucuggggacccaugggggaacu

SEQ ID NO: 100 - DNA, 21 bp, artificial
  synthetic aptamer
gccuggggacccauggggggc

SEQ ID NO: 101 - DNA, 23 bp, artificial
  synthetic aptamer
agccuggggacccauggggggcu

SEQ ID NO: 102 - DNA, 25 bp, artificial
  synthetic aptamer
uagccuggggacccauggggggcua

SEQ ID NO: 103 - DNA, 21 bp, artificial
  synthetic aptamer
gccuggggaaccauggggggc

SEQ ID NO: 104 - DNA, 68 bp, artificial
  synthetic template
gggagaggagagaacgttctacagcctggggacccatggggggctggtcgatcgatcgatcatcgatg

SEQ ID NO: 105 - DNA, 23 bp, artificial
  synthetic primer
catcgatgatcgatcgatcgacc

SEQ ID NO: 106 - DNA, 22 bp, artificial
  synthetic aptamer
agccuggggacccaugggggcu

SEQ ID NO: 107 - DNA, 23 bp, artificial
  synthetic aptamer
cgccuggggacccagggggggcu

SEQ ID NO: 108 - DNA, 23 bp, artificial
  synthetic aptamer
agccugguggcccauggggugcu

SEQ ID NO: 109 - DNA, 25 bp, artificial
  synthetic aptamer
agccuggggacccaugggggguggu

SEQ ID NO: 110 - DNA, 23 bp, artificial
  synthetic aptamer
agucuggggacagauggauggcu

SEQ ID NO: 111 - DNA, 22 bp, artificial
  synthetic aptamer
agcuguggagucguguggggcu

SEQ ID NO: 112 - DNA, 25 bp, artificial
  synthetic aptamer
aagccuggggacccaugggggggcu

SEQ ID NO: 113 - DNA, 37 bp, artificial
  synthetic aptamer
ggggcacgtttatccgtccctcctagtggcgtgcccc

SEQ ID NO: 114 - DNA, 74 bp, artificial
  synthetic template
gatcccttgttcagtccggggcacgtttatccgtccctcctagtggcgtgccccttaagccacaggactccaaa

SEQ ID NO: 115 - DNA, 18 bp, artificial
  synthetic primer
gatcccttgttcagtccg

SEQ ID NO: 116 - DNA, 17 bp, artificial
  synthetic primer
ggagtcctgtggcttaa

SEQ ID NO: 117 - DNA, 20 bp, artificial
  synthetic primer
nnnggagtcctgtggcttaa

SEQ ID NO: 118 - DNA, 17 bp, synthetic primer
ggagtcctgtggcttaa

SEQ ID NO: 119 - DNA, 37 bp, artificial
  synthetic aptamer
ggggcacatttatccgtccctcctagtggtgtgcccc

SEQ ID NO: 120 - DNA, 37 bp, artificial
  synthetic aptamer
ggggtacctttatccgtccctcctagtggggtgcccc

SEQ ID NO: 121 - DNA, 37 bp, artificial
  synthetic aptamer
ggggtacctttatccgtccctcctagtggggtacccc

SEQ ID NO: 122 - DNA, 37 bp, artificial
  synthetic aptamer
ggggcaaatttatccgtccctcctagtggtttgcccc

SEQ ID NO: 123 - DNA, 37 bp, artificial
  synthetic aptamer
ggggcatatttatccgtccctcctagtggtatgcccc

SEQ ID NO: 124 - DNA, 37 bp, artificial
  synthetic aptamer
ggggcacatttatccgttcctcctagtggtgtgcccc

SEQ ID NO: 125 - DNA, 37 bp, artificial
  synthetic aptamer
ggggtacatttatccgtccctcctagtggcatgcccc

SEQ ID NO: 126 - DNA, 37 bp, artificial
  synthetic aptamer
ggggcatgtttatccgtccctcctagtggcatgcccc

SEQ ID NO: 127 - DNA, 36 bp, artificial
  synthetic aptamer
ggggcaactttatccgttcctcctagtgggttgccc

SEQ ID NO: 128 - DNA, 37 bp, artificial
  synthetic aptamer
ggggcacattcatccgtccctcctagtggtgtgctcc

SEQ ID NO: 129 - DNA, 37 bp, artificial
  synthetic aptamer
ggggtaccttgatccgtccctcctagtggggtgcccc

SEQ ID NO: 130 - DNA, 37 bp, artificial
  synthetic aptamer
ggggcatgtttatccgttcctcctagtggcatgcccc

SEQ ID NO: 131 - DNA, 37 bp, artificial
  synthetic aptamer
ggggcagctttatccgttcctcctagtgggctgcctc

SEQ ID NO: 132 - DNA, 37 bp, artificial
  synthetic aptamer
ggggtacctttatccgtttctcctagtggggtgcccc

SEQ ID NO: 133 - DNA, 37 bp, artificial
  synthetic aptamer
ggggtatgttgatccgtccctcctagtggcatgcccc

SEQ ID NO: 134 - DNA, 37 bp, artificial
  synthetic aptamer
ggggcatgttcatccgttcctcctagtggcgtgcccc

SEQ ID NO: 135 - DNA, 37 bp, artificial
  synthetic aptamer
gggacacatttatccgttactcttagtggtgtgcccc

SEQ ID NO: 136 - DNA, 37 bp, artificial
  synthetic aptamer
ggggcacatttatccgttactcttagtggtgtgcccc

SEQ ID NO: 137 - DNA, 34 bp, artificial
  synthetic aptamer
ggggcacgtttacagtccctccttatcgcctccc

SEQ ID NO: 138 - DNA, 34 bp, artificial; identical to SEQ ID NO: 137
  synthetic aptamer

SEQ ID NO: 139 - DNA, 36 bp, artificial
  synthetic aptamer
gggcaactttatccgttcctcttagtgggttgcccc

SEQ ID NO: 140 - DNA, 36 bp, artificial
  synthetic aptamer
gggctactttatccgtccctcctagtgggtagcccc

SEQ ID NO: 141 - DNA, 35 bp, artificial
  synthetic aptamer
ggcacctttatccgtccctcctagtggggtgcccc

SEQ ID NO: 142 - DNA, 37 bp, artificial
  synthetic aptamer
ggggcacctttatccgtccctcctagtggggtgcccc

SEQ ID NO: 143 - DNA, 36 bp, artificial
  synthetic aptamer
gggcacattcatccgttcctcctagtggtgtgcccc

SEQ ID NO: 144 - DNA, 34 bp, artificial
  synthetic aptamer
ggcacctttatccgttccttctagtggggtgccc

SEQ ID NO: 145 - DNA, 36 bp, artificial
  synthetic aptamer
cggcacctttatccgttactcttagtgnggtgcccc

SEQ ID NO: 146 - DNA, 35 bp, artificial
  synthetic aptamer
ggcaccttgatccgttcctcctagtggggtgcccc

SEQ ID NO: 147 - DNA, 37 bp, artificial
  synthetic aptamer
gcgggcaaattcatccgtccctcctagtggtttgccc

SEQ ID NO: 148 - DNA, 34 bp, artificial
  synthetic aptamer
gggcactttatccgttccttctagtgggtgtccc

SEQ ID NO: 149 - DNA, 38 bp, artificial
  synthetic aptamer
ggcggcagctttatccgtacctcccagtgggctgctcc

SEQ ID NO: 150 - DNA, 37 bp, artificial
  synthetic aptamer
ggggcagctttatccgtacctcccagtgggctgcccc

SEQ ID NO: 151 - DNA, 37 bp, artificial
  synthetic aptamer
ggggctactttatccgtccctcctagtgggtagcccc

SEQ ID NO: 152 - DNA, 37 bp, artificial
  synthetic aptamer
ggggctactttatccgtacctcccagtgggtagcccc

SEQ ID NO: 153 - DNA, 37 bp, artificial
  synthetic aptamer
ggggctacttgatccgtccctcctagtgggtagcccc

SEQ ID NO: 154 - DNA, 37 bp, artificial
  synthetic aptamer
ggggctacttcatccgtccctcctagtgggtagcccc

SEQ ID NO: 155 - DNA, 37 bp, artificial
  synthetic aptamer
ggggctactttatccgttcctcttagtgggtagcccc

SEQ ID NO: 156 - DNA, 37 bp, artificial
  synthetic aptamer
ggggctactttatccgttcctcctagtgggtagcccc

SEQ ID NO: 157 - DNA, 38 bp, artificial
  synthetic aptamer
ggggctactttatccgttcctcctagtgggtagccccn

SEQ ID NO: 158 - DNA, 24 bp, artificial
  synthetic aptamer
agccuggggacccauggggggcun

SEQ ID NO: 159 - DNA, 24 bp, artificial
  synthetic aptamer
agccuggggacccauggggggcun

SEQ ID NO: 160 - DNA, 24 bp, artificial
  synthetic aptamer
agccuggggacccauggggggcun

SEQ ID NO: 161 - DNA, 24 bp, artificial
  synthetic aptamer
agccuggggacccauggggggcun

SEQ ID NO: 162 - DNA, 24 bp, artificial
  synthetic aptamer
agccuggggacccauggggggcun

SEQ ID NO: 163 - DNA, 24 bp, artificial
  synthetic aptamer
agccuggggacccauggggggcun

SEQ ID NO: 164 - DNA, 24 bp, artificial
  synthetic aptamer
agccuggggacccauggggggcun

SEQ ID NO: 165 - DNA, 24 bp, artificial
  synthetic aptamer
agccuggggacccauggggggcun

SEQ ID NO: 166 - DNA, 24 bp, artificial
  synthetic aptamer
agccuggggacccauggggggcun

SEQ ID NO: 167 - DNA, 24 bp, artificial
  synthetic aptamer
agccuggggacccauggggggcun

SEQ ID NO: 168 - DNA, 24 bp, artificial
  synthetic aptamer
agccuggggacccauggggggcun

SEQ ID NO: 169 - DNA, 24 bp, artificial
  synthetic aptamer
agccuggggacccauggggggcun

SEQ ID NO: 170 - DNA, 24 bp, artificial
  synthetic aptamer
agccuggggacccauggggggcun

SEQ ID NO: 171 - DNA, 24 bp, artificial
  synthetic aptamer
agccuggggacccauggggggcun

SEQ ID NO: 172 - DNA, 24 bp, artificial
  synthetic aptamer
agccuggggacccauggggggcun

SEQ ID NO: 173 - DNA, 24 bp, artificial
  synthetic aptamer
agccuggggacccauggggggcun

SEQ ID NO: 174 - DNA, 24 bp, artificial
  synthetic aptamer
agccuggggacccauggggggcun

SEQ ID NO: 175 - DNA, 24 bp, artificial
  synthetic aptamer
agccuggggacccauggggggcun

SEQ ID NO: 176 - DNA, 24 bp, artificial
  synthetic aptamer
agccuggggacccauggggggcun

SEQ ID NO: 177 - DNA, 28 bp, artificial
  synthetic aptamer
angccuggggacccaungnggggngcun

SEQ ID NO: 178 - DNA, 30 bp, artificial
  synthetic aptamer
angccuggggnacccaungnggnggngcun

SEQ ID NO: 179 - DNA, 27 bp, artificial
  synthetic aptamer
agccugggngacccaugggnggngcun

SEQ ID NO: 180 - DNA, 28 bp, artificial
  synthetic aptamer
agccugggngacccaungggnggngcun

SEQ ID NO: 181 - DNA, 29 bp, artificial
  synthetic aptamer
agccugggngacccaungnggnggngcun

SEQ ID NO: 182 - DNA, 32 bp, artificial
  synthetic aptamer
agccunggngngacccaunggngngngngcun

SEQ ID NO: 183 - DNA, 35 bp, artificial
  synthetic aptamer
agccungngngngacccaungngngngngngncun

SEQ ID NO: 184 - DNA, 23 bp, artificial
  synthetic aptamer
anccuggggacccauggggggcu

SEQ ID NO: 185 - DNA, 23 bp, artificial
  synthetic aptamer
agccungggacccauggggggcu

SEQ ID NO: 186 - DNA, 23 bp, artificial
  synthetic aptamer
agccugnggacccauggggggcu

SEQ ID NO: 187 - DNA, 23 bp, artificial
  synthetic aptamer
agccuggngacccauggggggcu

SEQ ID NO: 188 - DNA, 23 bp, artificial
  synthetic aptamer
agccugggnacccauggggggcu

SEQ ID NO: 189 - DNA, 23 bp, artificial
  synthetic aptamer
agccuggggacccaungggggcu

SEQ ID NO: 190 - DNA, 23 bp, artificial
  synthetic aptamer
agccuggggacccaugnggggcu

SEQ ID NO: 191 - DNA, 23 bp, artificial
  synthetic aptamer
agccuggggacccauggngggcu

SEQ ID NO: 192 - DNA, 23 bp, artificial
  synthetic aptamer
agccuggggacccaugggnggcu

SEQ ID NO: 193 - DNA, 23 bp, artificial
  synthetic aptamer
agccuggggacccauggggngcu

SEQ ID NO: 194 - DNA, 23 bp, artificial
  synthetic aptamer
agccuggggacccaugggggncu

SEQ ID NO: 195 - DNA, 23 bp, artificial
  synthetic aptamer
aggcuggggacccaugggggccu

SEQ ID NO: 196 - DNA, 23 bp, artificial
  synthetic aptamer
aggcuggggacccaugggggccu

SEQ ID NO: 197 - DNA, 23 bp, artificial
  synthetic aptamer
agucuggggacccaugggggacu

SEQ ID NO: 198 - DNA, 23 bp, artificial
  synthetic aptamer
agucuggggacccaugggggacu

SEQ ID NO: 199 - DNA, 23 bp, artificial
  synthetic aptamer
agacuggggacccaugggggucu

SEQ ID NO: 200 - DNA, 23 bp, artificial
  synthetic aptamer
agacuggggacccaugggggucu

SEQ ID NO: 201 - DNA, 24 bp, artificial
  synthetic aptamer
anccuggggacccauggggggcun

SEQ ID NO: 202 - DNA, 24 bp, artificial
  synthetic aptamer
agccungggacccauggggggcun

SEQ ID NO: 203 - DNA, 24 bp, artificial
  synthetic aptamer
agccugnggacccauggggggcun

SEQ ID NO: 204 - DNA, 24 bp, artificial
  synthetic aptamer
agccuggngacccauggggggcun

SEQ ID NO: 205 - DNA, 24 bp, artificial
  synthetic aptamer
agccugggnacccauggggggcun

SEQ ID NO: 206 - DNA, 24 bp, artificial
  synthetic aptamer
agccuggggacccaungggggcun

SEQ ID NO: 207 - DNA, 24 bp, artificial
  synthetic aptamer
agccuggggacccaugnggggcun

SEQ ID NO: 208 - DNA, 24 bp, artificial
  synthetic aptamer
agccuggggacccauggngggcun

SEQ ID NO: 209 - DNA, 24 bp, artificial
  synthetic aptamer
agccuggggacccaugggnggcun

SEQ ID NO: 210 - DNA, 24 bp, artificial
  synthetic aptamer
agccuggggacccauggggngcun

SEQ ID NO: 211 - DNA, 24 bp, artificial
  synthetic aptamer
agccuggggacccaugggggncun

SEQ ID NO: 212 - DNA, 29 bp, artificial
  synthetic aptamer
agccugggngacccaungnggngnngcun

SEQ ID NO: 213 - DNA, 29 bp, artificial
  synthetic aptamer
agccugggngacccaunnnggngnngcun

SEQ ID NO: 214 - DNA, 32 bp, artificial
  synthetic aptamer
agccunggngngacccaunggngngnnngcun

SEQ ID NO: 215 - DNA, 32 bp, artificial
  synthetic aptamer
agccunggngngacccaunngngngnnngcun

SEQ ID NO: 216 - DNA, 29 bp, artificial
  synthetic aptamer
agccugggngacccaunnngnngnngcun

SEQ ID NO: 217 - DNA, 29 bp, artificial
  synthetic aptamer
agccugggngacccaungngnngnngcun

SEQ ID NO: 218 - DNA, 32 bp, artificial
  synthetic aptamer
agccunggngngacccaunggnnngnnngcun

SEQ ID NO: 219 - DNA, 32 bp, artificial
  synthetic aptamer
agccunggngngacccaunngnnngnnngcun

SEQ ID NO: 220 - DNA, 38 bp, artificial; identical to SEQ ID NO: 157
  synthetic aptamer

SEQ ID NO: 221 - DNA, 38 bp, artificial; identical to SEQ ID NO: 157
  synthetic aptamer

SEQ ID NO: 222 - DNA, 38 bp, artificial; identical to SEQ ID NO: 157
  synthetic aptamer

SEQ ID NO: 223 - DNA, 38 bp, artificial; identical to SEQ ID NO: 157
  synthetic aptamer

SEQ ID NO: 224 - DNA, 38 bp, artificial; identical to SEQ ID NO: 157
  synthetic aptamer

SEQ ID NO: 225 - DNA, 38 bp, artificial
  synthetic aptamer
ggggcuactttatccgttcctcctagtgggtagccccn

SEQ ID NO: 226 - DNA, 38 bp, artificial; identical to SEQ ID NO: 157
  synthetic aptamer

SEQ ID NO: 227 - DNA, 38 bp, artificial; identical to SEQ ID NO: 157
  synthetic aptamer

SEQ ID NO: 228 - DNA, 38 bp, artificial
  synthetic aptamer
ggggctacuttatccgttcctcctagtgggtagccccn

SEQ ID NO: 229 - DNA, 38 bp, artificial
  synthetic aptamer
ggggctactutatccgttcctcctagtgggtagccccn

SEQ ID NO: 230 - DNA, 38 bp, artificial
  synthetic aptamer
ggggctacttuatccgttcctcctagtgggtagccccn

SEQ ID NO: 231 - DNA, 38 bp, artificial; identical to SEQ ID NO: 157
  synthetic aptamer

SEQ ID NO: 232 - DNA, 38 bp, artificial
  synthetic aptamer
ggggctactttauccgttcctcctagtgggtagccccn

SEQ ID NO: 233 - DNA, 38 bp, artificial; identical to SEQ ID NO: 157
  synthetic aptamer

SEQ ID NO: 234 - DNA, 38 bp, artificial; identical to SEQ ID NO: 157
  synthetic aptamer

SEQ ID NO: 235 - DNA, 38 bp, artificial; identical to SEQ ID NO: 157
  synthetic aptamer

SEQ ID NO: 236 - DNA, 38 bp, artificial
  synthetic aptamer
ggggctactttatccgutcctcctagtgggtagccccn

SEQ ID NO: 237 - DNA, 38 bp, artificial
  synthetic aptamer
ggggctactttatccgtucctcctagtgggtagccccn

SEQ ID NO: 238 - DNA, 38 bp, artificial; identical to SEQ ID NO: 157
  synthetic aptamer

SEQ ID NO: 239 - DNA, 38 bp, artificial; identical to SEQ ID NO: 157
  synthetic aptamer

SEQ ID NO: 240 - DNA, 38 bp, artificial
  synthetic aptamer
ggggctactttatccgttccucctagtgggtagccccn

SEQ ID NO: 241 - DNA, 38 bp, artificial; identical to SEQ ID NO: 157
  synthetic aptamer

SEQ ID NO: 242 - DNA, 38 bp, artificial; identical to SEQ ID NO: 157
  synthetic aptamer

SEQ ID NO: 243 - DNA, 38 bp, artificial
  synthetic aptamer
ggggctactttatccgttcctccuagtgggtagccccn

SEQ ID NO: 244 - DNA, 38 bp, artificial; identical to SEQ ID NO: 157
  synthetic aptamer

SEQ ID NO: 245 - DNA, 38 bp, artificial; identical to SEQ ID NO: 157
  synthetic aptamer

SEQ ID NO: 246 - DNA, 38 bp, artificial
  synthetic aptamer
ggggctactttatccgttcctcctagugggtagccccn

SEQ ID NO: 247 - DNA, 38 bp, artificial; identical to SEQ ID NO: 157
  synthetic aptamer

SEQ ID NO: 248 - DNA, 38 bp, artificial; identical to SEQ ID NO: 157
  synthetic aptamer

SEQ ID NO: 249 - DNA, 38 bp, artificial; identical to SEQ ID NO: 157
  synthetic aptamer

SEQ ID NO: 250 - DNA, 38 bp, artificial
  synthetic aptamer
ggggctactttatccgttcctcctagtggguagccccn

SEQ ID NO: 251 - DNA, 38 bp, artificial; identical to SEQ ID NO: 157
  synthetic aptamer

SEQ ID NO: 252 - DNA, 38 bp, artificial; identical to SEQ ID NO: 157
  synthetic aptamer

SEQ ID NO: 253 - DNA, 38 bp, artificial; identical to SEQ ID NO: 157
  synthetic aptamer

SEQ ID NO: 254 - DNA, 38 bp, artificial; identical to SEQ ID NO: 157
  synthetic aptamer

SEQ ID NO: 255 - DNA, 38 bp, artificial; identical to SEQ ID NO: 157
  synthetic aptamer

SEQ ID NO: 256 - DNA, 38 bp, artificial; identical to SEQ ID NO: 157
  synthetic aptamer

SEQ ID NO: 257 - DNA, 38 bp, artificial; identical to SEQ ID NO: 157
  synthetic aptamer

SEQ ID NO: 258 - DNA, 38 bp, artificial
  synthetic aptamer
ggggcuactttatccgttcctcctagtggguagccccn

SEQ ID NO: 259 - DNA, 38 bp, artificial; identical to SEQ ID NO: 157
  synthetic aptamer

SEQ ID NO: 260 - DNA, 38 bp, artificial; identical to SEQ ID NO: 157
  synthetic aptamer

SEQ ID NO: 261 - DNA, 36 bp, artificial
  synthetic aptamer
gggctactttatccgttcctcctagtgggtagcccn

SEQ ID NO: 262 - DNA, 38 bp, artificial; identical to SEQ ID NO: 157
  synthetic aptamer

SEQ ID NO: 263 - DNA, 38 bp, artificial; identical to SEQ ID NO: 225
  synthetic aptamer

SEQ ID NO: 264 - DNA, 38 bp, artificial
  syjnthetic aptamer
ggggctacttuatccguucctcctagtgggtagccccn

SEQ ID NO: 265 - DNA, 38 bp, artificial
  synthetic aptamer
ggggcuacttuatccguucctcctagtgggtagccccn

SEQ ID NO: 266 - DNA, 39 bp, artificial
  synthetic aptamer
ggggctacntttatccgttcctcctagtgggtagccccn

SEQ ID NO: 267 - DNA, 39 bp, artificial
  synthetic aptamer
ggggctactnttatccgttcctcctagtgggtagccccn

SEQ ID NO: 268 - DNA, 39 bp, artificial
  synthetic aptamer
ggggctacttntatccgttcctcctagtgggtagccccn

SEQ ID NO: 269 - DNA, 39 bp, artificial
  synthetic aptamer
ggggctactttnatccgttcctcctagtgggtagccccn

SEQ ID NO: 270 - DNA, 39 bp, artificial
  synthetic aptamer
ggggctactttantccgttcctcctagtgggtagccccn

SEQ ID NO: 271 - DNA, 39 bp, artificial
  synthetic aptamer
ggggctactttatnccgttcctcctagtgggtagccccn

SEQ ID NO: 272 - DNA, 39 bp, artificial
  synthetic aptamer
ggggctactttatcncgttcctcctagtgggtagccccn

SEQ ID NO: 273 - DNA, 39 bp, artificial
  synthetic aptamer
ggggctactttatccngttcctcctagtgggtagccccn

SEQ ID NO: 274 - DNA, 39 bp, artificial
  synthetic aptamer
ggggctactttatccgnttcctcctagtgggtagccccn

SEQ ID NO: 275 - DNA, 39 bp, artificial
  synthetic aptamer
ggggctactttatccgtntcctcctagtgggtagccccn

SEQ ID NO: 276 - DNA, 39 bp, artificial
  synthetic aptamer
ggggctactttatccgttncctcctagtgggtagccccn

SEQ ID NO: 277 - DNA, 39 bp, artificial
  synthetic aptamer
ggggctactttatccgttcnctcctagtgggtagccccn

SEQ ID NO: 278 - DNA, 39 bp, artificial
  synthetic aptamer
ggggctactttatccgttccntcctagtgggtagccccn

SEQ ID NO: 279 - DNA, 39 bp, artificial
  synthetic aptamer
ggggctactttatccgttcctncctagtgggtagccccn

SEQ ID NO: 280 - DNA, 39 bp, artificial
  synthetic aptamer
ggggctactttatccgttcctcnctagtgggtagccccn

SEQ ID NO: 281 - DNA, 39 bp, artificial
  synthetic aptamer
ggggctactttatccgttcctccntagtgggtagccccn

SEQ ID NO: 282 - DNA, 39 bp, artificial
  synthetic aptamer
ggggctactttatccgttcctcctnagtgggtagccccn

SEQ ID NO: 283 - DNA, 39 bp, artificial
  synthetic aptamer
ggggctactttatccgttcctcctangtgggtagccccn

SEQ ID NO: 284 - DNA, 39 bp, artificial
  synthetic aptamer
ggggctactttatccgttcctcctagntgggtagccccn

SEQ ID NO: 285 - DNA, 39 bp, artificial
  synthetic aptamer
ggggctactttatccgttcctcctagtngggtagccccn

SEQ ID NO: 286 - DNA, 39 bp, artificial
  synthetic aptamer
ggggctactttatccgttcctcctagtgnggtagccccn

SEQ ID NO: 287 - DNA, 39 bp, artificial
  synthetic aptamer
ggggctactttatccgttcctcctagtggngtagccccn

SEQ ID NO: 288 - DNA, 39 bp, artificial
  synthetic aptamer
ggggctactttatccgttcctcctagtgggntagccccn

SEQ ID NO: 289 - DNA, 39 bp, artificial
  synthetic aptamer
ggggctactttatccgttcctcctagtgggtnagccccn

SEQ ID NO: 290 - DNA, 46 bp, artificial
  synthetic aptamer
ggggctactnttantcncgttncctncctnagtngggntagccccn

SEQ ID NO: 291 - DNA, 46 bp, artificial
  synthetic aptamer
ggggcuactnttantcncgttncctncctnagtngggntagccccn

SEQ ID NO: 292 - DNA, 45 bp, artificial
  synthetic aptamer
ggggcuactntuantcncguucctncctnagtngggntagccccn

SEQ ID NO: 293 - DNA, 31 bp, artificial
  synthetic aptamer
nnagccugggngacccaunnngnngnngcun

SEQ ID NO: 294 - DNA, 31 bp, artificial; identical to SEQ ID NO: 293
  synthetic aptamer

SEQ ID NO: 295 - DNA, 32 bp, artificial
  synthetic aptamer
nnagccugggngacccaunnngnngnngcunn

SEQ ID NO: 296 - DNA, 32 bp, artificial; identical to SEQ ID NO: 295
  synthetic aptamer

SEQ ID NO: 297 - DNA, 30 bp, artificial
  synthetic aptamer
nagccugggngacccaunnngnngnngcun

SEQ ID NO: 298 - DNA, 28 bp, artificial
  synthetic aptamer
agccugggngacccaunnngnngnngcu

SEQ ID NO: 299 - DNA, 30 bp, Artificial
  synthetic aptamer
agccugggngacccaunnngnngnngncun

SEQ ID NO: 300 - DNA, 31 bp, Artificial
  synthetic aptamer
agccugggngacccaunnngnngnngncnun

SEQ ID NO: 301 - DNA, 32 bp, Artificial
  synthetic aptamer
agccugggngacccaunnngnngnngncnunn

SEQ ID NO: 302 - DNA, 30 bp, Artificial
  synthetic aptamer
agccugggngacccaunnngnngnngcunn

SEQ ID NO: 303 - DNA, 29 bp, Artificial
  synthetic aptamer
agccugggngacccaunnngnngnngcun

SEQ ID NO: 304 - DNA, 30 bp, Artificial
  synthetic aptamer
angccugggngacccaunnngnngnngcun

SEQ ID NO: 305 - DNA, 30 bp, Artificial
  synthetic aptamer
agnccugggngacccaunnngnngnngcun

SEQ ID NO: 306 - DNA, 30 bp, Artificial
  synthetic aptamer
agcncugggngacccaunnngnngnngcun

SEQ ID NO: 307 - DNA, 30 bp, Artificial
  synthetic aptamer
agccnugggngacccaunnngnngnngcun

SEQ ID NO: 308 - DNA, 30 bp, Artificial
  synthetic aptamer
agccungggngacccaunnngnngnngcun

SEQ ID NO: 309 - DNA, 30 bp, Artificial
  synthetic aptamer
agccugnggngacccaunnngnngnngcun

SEQ ID NO: 310 - DNA, 30 bp, Artificial
  synthetic aptamer
agccuggngngacccaunnngnngnngcun

SEQ ID NO: 311 - DNA, 30 bp, Artificial
  synthetic aptamer
agccugggngnacccaunnngnngnngcun

SEQ ID NO: 312 - DNA, 30 bp, Artificial
  synthetic aptamer
agccugggngancccaunnngnngnngcun

SEQ ID NO: 313 - DNA, 30 bp, Artificial
  synthetic aptamer
agccugggngacnccaunnngnngnngcun

SEQ ID NO: 314 - DNA, 30 bp, Artificial
  synthetic aptamer
agccugggngaccncaunnngnngnngcun

SEQ ID NO: 315 - DNA, 30 bp, Artificial
  synthetic aptamer
agccugggngacccnaunnngnngnngcun

SEQ ID NO: 316 - DNA, 30 bp, Artificial
  synthetic aptamer
agccugggngacccanunnngnngnngcun

SEQ ID NO: 317 - DNA, 30 bp, Artificial
  synthetic aptamer
agccugggngacccaunnngnnngnngcun

SEQ ID NO: 318 - DNA, 30 bp, Artificial
  synthetic aptamer
agccugggngacccaunnngnngnnngcun

SEQ ID NO: 319 - DNA, 29 bp, Artificial
  synthetic aptamer
agccugggngacccaunnngnngnngcun

SEQ ID NO: 320 - DNA, 29 bp, Artificial
  synthetic aptamer
agccugggngacccaunnngnngnngcun

SEQ ID NO: 321 - DNA, 29 bp, Artificial
  synthetic aptamer
agccugggngacccaunnngnngnngcun

SEQ ID NO: 322 - DNA, 29 bp, Artificial
  synthetic aptamer
agccugggngacccaunnngnngnngcun

SEQ ID NO: 323 - DNA, 29 bp, Artificial
  synthetic aptamer
agccugggngacccaunnngnngnngcun

SEQ ID NO: 324 - DNA, 29 bp, Artificial
  synthetic aptamer
agccugggngacccaunnngnngnngcun

SEQ ID NO: 325 - DNA, 29 bp, Artificial
  synthetic aptamer
agccugggngacccaunnngnngnngcun

SEQ ID NO: 326 - DNA, 32 bp, Artificial; identical to SEQ ID NO: 301
  synthetic aptamer

SEQ ID NO: 327 - DNA, 32 bp, Artificial; identical to SEQ ID NO: 301
  synthetic aptamer

SEQ ID NO: 328 - DNA, 32 bp, Artificial; identical to SEQ ID NO: 301
  synthetic aptamer

SEQ ID NO: 329 - DNA, 32 bp, Artificial; identical to SEQ ID NO: 301
  synthetic aptamer

SEQ ID NO: 330 - DNA, 32 bp, Artificial; identical to SEQ ID NO: 301
  synthetic aptamer

SEQ ID NO: 331 - DNA, 32 bp, Artificial; identical to SEQ ID NO: 301
  synthetic aptamer

SEQ ID NO: 332 - DNA, 32 bp, Artificial; identical to SEQ ID NO: 301
  synthetic aptamer

SEQ ID NO: 333 - DNA, 35 bp, Artificial
  synthetic aptamer
aguagccugggngacccaunnngnngnngcuacun

SEQ ID NO: 334 - DNA, 28 bp, Artificial
  synthetic aptamer
agccugggngacccaunnngnngnngcu

SEQ ID NO: 335 - DNA, 27 bp, Artificial
  synthetic aptamer
agccugggngacccaunnngnngnngc

SEQ ID NO: 336 - DNA, 26 bp, Artificial
  synthetic aptamer
agccugggngacccaunnngnngnng